SEQUENCE LISTING (20 sequences)

SEQ ID NO: 1 - protein, 12 aa, synthetic construct
  REGION 1..12: note = Synthetic peptide
GPLGLAGGERDG

SEQ ID NO: 2 - protein, 12 aa, synthetic construct
  REGION 1..12: note = Synthetic peptide; note = All Amino Acids are D-amino acids
GPLGLAGGERDG

SEQ ID NO: 3 - protein, 12 aa, synthetic construct
  REGION 1..12: note = Synthetic peptide
  SITE 1: note = misc_feature - Xaa can be any naturally occurring amino acid
  REGION 8..12: note = misc_feature - Xaa can be any naturally occurring amino acid
XPLGLAGXXXXX

SEQ ID NO: 4 - protein, 15 aa, synthetic construct
  REGION 1..15: note = Synthetic peptide
GPLGLAGGWGERDGS

SEQ ID NO: 5 - protein, 11 aa, synthetic construct
  REGION 1..11: note = Synthetic peptide
LAGGWGERDGS

SEQ ID NO: 6 - protein, 7 aa, synthetic construct
  REGION 1..7: note = Synthetic peptide
GGSGSGS

SEQ ID NO: 7 - protein, 7 aa, synthetic construct
  REGION 1..7: note = Synthetic peptide
GGSGSGE

SEQ ID NO: 8 - protein, 7 aa, synthetic construct
  REGION 1..7: note = Synthetic peptide
GGSGSGK

SEQ ID NO: 9 - protein, 7 aa, synthetic construct
  REGION 1..7: note = Synthetic peptide
GGSGSGR

SEQ ID NO: 10 - protein, 8 aa, synthetic construct
  REGION 1..8: note = Synthetic peptide
GGSGSGRR

SEQ ID NO: 11 - protein, 9 aa, synthetic construct
  REGION 1..9: note = Synthetic peptide
KVPRNQDWL

SEQ ID NO: 12 - protein, 7 aa, synthetic construct
  REGION 1..7: note = Synthetic peptide
GPLGLAG

SEQ ID NO: 13 - protein, 7 aa, synthetic construct
  REGION 1..7: note = Synthetic peptide
LRRASLG

SEQ ID NO: 14 - protein, 8 aa, synthetic construct
  REGION 1..8: note = Synthetic peptide
GPLGLAGK

SEQ ID NO: 15 - protein, 7 aa, synthetic construct
  REGION 1..7: note = Synthetic peptide
GPLGLAL

SEQ ID NO: 16 - protein, 6 aa, synthetic construct
  REGION 1..6: note = Synthetic peptide
PLGLAG

SEQ ID NO: 17 - protein, 15 aa, synthetic construct
  REGION 1..15: note = Synthetic peptide
GGSGSGSGWGERDGS

SEQ ID NO: 18 - protein, 11 aa, synthetic construct
  REGION 1..11: note = Synthetic peptide
LAGGWGERDGS

SEQ ID NO: 19 - protein, 15 aa, synthetic construct
  REGION 1..15: note = Synthetic peptide
  REGION 2..7: note = D-amino acids
GPLGLAGGWGERDGS

SEQ ID NO: 20 - protein, 6 aa, synthetic construct
  REGION 1..6: note = Synthetic peptide
GSGSGS

(19) United States
(12) Patent Application Publication
(10) Pub. No.: US 2024/0293316 A1
(43) Pub. Date: Sep. 5, 2024

(54) ENZYME-RESPONSIVE POLYMER PEPTIDE AMPHIPHILES FOR TARGETED DRUG DELIVERY TO TREAT LOCAL AND SYSTEMIC INFLAMMATION

(71) Applicants: Ramot at Tel-Aviv University Ltd., Tel-Aviv (IL); Northwestern University, Evanston, IL (US)

(72) Inventors: Nathan GIANNESCHI, Evanston, IL (US); Yifei LIANG, Evanston, IL (US); Claudia Battistella, Evanston, IL (US); Lihi ADLER-ABRAMOVICH, Tel-Aviv (IL); Eyal ROSEN, Tel-Aviv (IL); Carlos NEMCOVSKY, Tel-Aviv (IL); Nathan SCHIFFMANN, Tel-Aviv (IL); Michal HALPERIN-STERNFELD, Tel-Aviv (IL)

(73) Assignees: Ramot at Tel-Aviv University Ltd., Tel-Aviv (IL); Northwestern University, Evanston, IL (US)

(21) Appl. No.: 18/415,811

(22) Filed: Jan. 18, 2024

Related U.S. Application Data
(63) Continuation of application No. PCT/IL2022/050780, filed on Jul. 19, 2022.
(60) Provisional application No. 63/223,354, filed on Jul. 19, 2021.

(51) Int. Cl. A61K 9/107; A61K 47/59

(52) U.S. Cl. CPC A61K 9/1075; A61K 47/59

(57) ABSTRACT

Micellar particles formed of a plurality of block copolymers, compositions comprising same, copolymers used to form the particles, and polymeric aggregates formed therefrom are provided. The micellar particles are for use in the treatment of inflammation, including chronic inflammation. The block copolymers forming the particles include at least a first block and a second block. The first block has an anti-inflammatory agent covalently attached, directly or via a linking moiety or group, to at least a portion of its backbone units; and the second block has a hydrophilic moiety that comprises an amino acid sequence that is capable of interacting with an inflammatory biomarker covalently attached to at least a portion of its backbone units, and the particle features a core made of the first block and a hydrophilic shell made of the second block.

### RELATED APPLICATIONS

This application is a Continuation of PCT Patent Application No. PCT/IL2022/050780 having International filing date of Jul. 19, 2022, which claims the benefit of priority of under 35 USC § 119(e) of U.S. Provisional Patent Application No. 63/223,354 filed on Jul. 19, 2021. The contents of the above applications are all incorporated by reference as if fully set forth herein in their entirety.

### STATEMENT REGARDING FEDERALLY SPONSORED RESEARCH OR DEVELOPMENT

This invention was made with Government support under Grant No. R01HL139001 awarded by the National Institutes of Health. The Government has certain rights in the invention.

### SEQUENCE LISTING STATEMENT

The XML file, entitled 98847Sequence Listing.xml, created on Jan. 18, 2024, comprising 26,409 bytes, submitted concurrently with the filing of this application is incorporated herein by reference.

### FIELD AND BACKGROUND OF THE INVENTION

The present invention, in some embodiments thereof, relates to therapy, and more particularly, but not exclusively, to novel block copolymers that comprise an anti-inflammatory agent-containing and a stimuli-responsive peptide-containing polymer amphiphilic materials (PPAs) and to micellar particles formed thereof which are usable, for example, in the treatment of inflammation.

Recently, stimuli-responsive or “smart” materials that respond to disease-associated triggers are emerging as a promising tool for targeted and sustained drug delivery to improve efficacy and reduce side effects.

In recent years, stimuli-responsive polymers have gained increasing attentions towards applications in drug delivery and tissue engineering. Such polymers and their use in various biomedical applications have bene described, for example, in Giannechi et al. Adv. Mater., 2021, 2007504; Zhen et al. Nat Biomed Eng., 2020, 4, 499-506; Johnson et al. Nat Commun., 2014, 5, 5460; Shieh et al. Nat. Chem., 2019, 11, 1124-1132; and Xiao et al. J. Mater. Chem. B, 2020, 8, 6697-6709.

Such polymers provide both active targeting to the diseased site by being responsive to a stimuli that is over-expressed or accumulated at the diseased site; and passive targeting due to the enhanced permeability and retention (EPR) effect that promotes the extravasation of macromolecules into abnormally leaky blood vessels in the disease site with respect to healthy tissues.

The use of enzyme-responsive peptide-polymer amphiphiles (PPAs) for minimally invasive delivery of drugs to diseased myocardial tissues, wherein PPAs were shown to aggregate in diseased tissue where matrix metalloproteinases (MMPs) are upregulated have been recently reported [see, Nguyen et al., Adv Mater, 2015, 27, 5547-5552; and Ungerleider et al., Polym Chem, 2017, 8, 5212-5219]. These PPAs have been formulated for intravenous (IV) injection or local injection, and target the inflamed tissues through the enhanced permeability and retention (EPR) effect associated with the diseased tissues due to leaky vasculature.

Peptide brush polymers were synthesized and their application in targeting cancer and myocardial infarction was demonstrated in Callmann et al. Adv. Mater. 2015, 27 (31) 4611-4615; Nguyen et al. 2015, supra; Kammeyer et al. Polym. Chem. 2013, 4 (14), 3929-3933; Sun et al. Angew. Chem. Int. Ed., 2019, 58, 17359-17364; U.S. Pat. No. 10,980,744; Battistella et al. Adv. Healthcare Mater. 2019, 8, 1901105; International Patent Publication No. WO 2016/172386; U.S. Pat. No. 9,040,626; U.S. Patent Application Publication Nos. 2014/0193342, 2017/0000909, 2018/0042843 and 2021/0283054; WO 2021/030326, WO 2021/222523, and WO 2022/006387.

High density PPAs for improving peptide cellular uptake have been described in Blum et al. J. Am. Chem. Soc. 2014, 136 (43), 15422-15437; and Blum et al. Chem. Sci. 2016, 7 (2), 989-994.

Background Art FIGS. 1A-C present schematically some of the recent developments in this field.

The self-assembly process of PPAs has been described in Wright et al. [Macromol. Rapid Commun. 2019, 40, 1800467].

Additional Background Art includes Schiffmann et al., “Enzyme-Responsive Nanoparticles for Targeted Drug Delivery to Inflamed Oral Tissues”, scientific poster, presented at the 2021 Israeli Division Meeting (Jerusalem, Israel, 25 Jun. 2021) and at the 2021 IADR/AADR/CADR General Session (Virtual Experience, 23 Jul. 2021).

### SUMMARY OF THE INVENTION

According to an aspect of some embodiments of the present invention there is provided a micellar particle for use in the treatment of chronic inflammation in a subject in need thereof, the micellar particle comprising a plurality of block copolymers, at least a portion, or each, of the block copolymers comprising at least one first block composed of a first plurality of backbone units covalently linked to one another and at least one second block of a second plurality of backbone units covalently linked to one another, wherein: the first block comprises at least one anti-inflammatory agent covalently attached, directly or via a linking moiety or group, to at least a portion of the first plurality of backbone units composing the first block; and the second block comprises at least one hydrophilic moiety covalently attached to at least a portion of the second plurality of backbone units composing the second block, the at least one hydrophilic moiety comprises an amino acid sequence that is capable of interacting with an inflammatory biomarker, the particle comprising a core comprising the first block and a hydrophilic shell comprising the second block.

According to some of any of the embodiments described herein, the hydrophilic moiety comprises an inflammatory protease-cleavable amino acid sequence.

According to some of any of the embodiments described herein, the hydrophilic moiety comprises an MMP-9-cleavable and/or an MMP-2-cleavable amino acid sequence.

According to some of any of the embodiments described herein, the block copolymer comprises at least two of the first blocks, wherein the at least one anti-inflammatory agent is the same or different in each of the at least two first blocks.

According to some of any of the embodiments described herein, the first block comprises a first anti-inflammatory drug attached to a first portion of backbone units in the first block and a second anti-inflammatory drug attached to a second portion of backbone units in the first block, wherein the first and second anti-inflammatory agents are different from one another.

According to some of any of the embodiments described herein, the block copolymer comprises at least two of the second blocks, wherein the at least one hydrophilic moiety is the same or different in each of the at least two second blocks.

According to some of any of the embodiments described herein, the second block comprises a first hydrophilic moiety attached to a first portion of backbone units in the second block and a second hydrophilic moiety attached to a second portion of backbone units in the second block, wherein the first and second hydrophilic moieties are different from one another. According to some of any of the embodiments described herein, the at least one anti-inflammatory agent is hydrophobic.

According to some of any of the embodiments described herein, the block copolymer further comprises at least one third block composed of a third plurality of backbone units covalently linked to one another, the third block comprises at least one additional moiety covalently attached, directly or via a linking moiety or group, to at least a portion of the third plurality of backbone units composing the third block, the additional moiety being a hydrophobic moiety that is different from the at least one anti-inflammatory agent or an additional hydrophilic moiety that is different from the hydrophilic moiety that comprises an amino acid sequence that is capable of interacting with an inflammatory biomarker.

According to some of any of the embodiments described herein, the block copolymer further comprises at least one backbone unit that has a labeling agent attached thereto.

According to some of any of the embodiments described herein, the at least one backbone unit is adjacent to the at least one second block.

According to some of any of the embodiments described herein, the block copolymer further comprises a forth block composed of a forth plurality of backbone units covalently linked to one another, the forth block comprises at least one labeling agent covalently attached, directly or via a linking moiety or group, to at least a portion of the forth plurality of backbone units composing the fourth block.

According to some of any of the embodiments described herein, the block copolymer is represented by Formula I as described herein in any of the respective embodiments and any combination thereof.

According to some of any of the embodiments described herein, each backbone unit of the first plurality of backbone units of the at least one first block, second plurality of backbone units of the at least one second block, third plurality of backbone units of the at least one third block, if present, and fourth plurality of backbone units of at least one the fourth block, if present, is independently a ROMP-polymerized monomer.

According to some of any of the embodiments described herein, the ROMP-polymerized monomer is derived from a substituted or unsubstituted norbornene.

According to some of any of the embodiments described herein, the anti-inflammatory agent is a small molecule compound.

According to some of any of the embodiments described herein, the anti-inflammatory agent is dexamethasone or a therapeutically active derivative thereof.

According to some of any of the embodiments described herein, the hydrophilic moiety comprises an amino acid sequence as set forth in SEQ ID NOS: 1, 3, 4, 12, 14, and 16.

According to some of any of the embodiments described herein, the micellar particle is formed upon contacting a plurality of the block copolymers with an aqueous solution.

According to some of any of the embodiments described herein, the micellar particle is capable of forming a polymeric aggregate upon contacting the inflammatory biomarker.

According to some of any of the embodiments described herein, the micellar particle is a micellar nanoparticle.

According to some of any of the embodiments described herein, the micellar particle forms a part of a pharmaceutical composition, which further comprises a pharmaceutically acceptable carrier.

According to some of any of the embodiments described herein, the carrier is an aqueous carrier.

According to some of any of the embodiments described herein, treating the chronic inflammation comprises locally administering the micellar particle or a pharmaceutical composition comprising same to an inflamed tissue and/or to a nearby vicinity thereof.

According to some of any of the embodiments described herein, treating the chronic inflammation comprises administering the micellar particle or a pharmaceutical composition comprising same once during a time period of at least one day, at least one week, or at least one month.

According to an aspect of some embodiments of the present invention there is provided a method of preparing the micellar particle as described herein in any of the respective embodiments and any combination thereof, the method comprising contacting the plurality of block copolymers with an aqueous solution.

According to an aspect of some embodiments of the present invention there is provided a block copolymer as described herein in any of the respective embodiments and any combination thereof.

According to an aspect of some embodiments of the present invention there is provided a pharmaceutical composition comprising a micellar particle as described herein in any of the respective embodiments and any combination thereof, or a plurality thereof, and a pharmaceutically acceptable carrier, for use in treating chronic inflammation in a subject in need thereof.

According to some of any of the embodiments described herein, the pharmaceutical composition is formulated for local administration at or in close vicinity to, an inflamed tissue in the subject.

According to some of any of the embodiments described herein, the pharmaceutical composition is formulated for local administration by injection.

According to some of any of the embodiments described herein, the carrier is an aqueous carrier.

According to an aspect of some embodiments of the present invention there is provided a polymeric aggregate formed upon contacting a plurality of micellar particles as described herein with an inflammatory marker, as described herein.

Other embodiments and aspects of the present invention are provided hereinbelow.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C (Background Art) present MMP-targeted PPAs. Background Art FIGS. 1A-B present PPAs labeled with a Rhodamine dye, assembled delivery vehicles formed therefrom, and the vehicles' targeted accumulation in an injured heart tissue, as a platform for the treatment of myocardial infarction, as described in Nguyen et al., Adv Mater, 2015, 27, 5547-5552; and Ungerleider et al., Polym Chem, 2017, 8, 5212-5219). Background Art FIG. 1C presents a summary of the chemotherapeutic and immunotherapeutic activity of PPAs having paclitaxel (PTX) conjugated thereto in rats, as described, for example, in Battistella et al. Adv. Healthcare Mater. 2019, 8, 1901105.

FIGS. 2A-B and Background Art FIG. 2C present an exemplary synthetic pathway of preparing exemplary drug-containing MMP-targeted/stimulated PPAs (e.g., block copolymers), according to some embodiments of the present invention and their assembly into a micellar particle (e.g., a nanoparticle) having a hydrophobic core and a hydrophilic (dye and MMP-peptide) shell (FIG. 2A); a schematic illustration of treating an exemplary inflammatory oral lesion using drug-containing MMP-targeted/stimulated micellar particles (e.g., nanoparticles), according to some embodiments of the present invention (FIG., 2B); and a schematic illustration of a healthy vs. diseased tooth (Background Art FIG. 2C).

FIGS. 3A-B present schematic illustrations of an exemplary study designed for evaluating an effect of exemplary PPA nanoparticles (NPs; micellar particles) in a surgical wound model (FIG. 3A) and the study timeline (FIG. 3B). The presented exemplary PPA is a block copolymer comprised of block(s) of a polymer comprising backbone units having an MMP-responsive peptide (purple) conjugated thereto (60%) and block(s) of a polymer comprising backbone units having an MMP-responsive peptide (purple) and a Cy5.5 fluorescent dye (green) conjugated thereto (40%), which self-assembles into enzyme-responsive fluorescent nanoparticles (micellar particles), and the latter are injected to an induced would in the oral cavity.

FIGS. 4A-S present data obtained in the study illustrated in FIGS. 3A-B, as images taken by black and white camera (Black & White; FIGS. 4A-D, 4I-L, and 4Q) and by in-vivo whole body fluorescence imaging (in-vivo fluorescence; FIGS. 4E-H, 4M-P, 4R, and 4S) of the studied rats, at days 3 (FIGS. 4A-H) and 10 (FIGS. 4I-P) of the experiment (2 and 9 days after the wound induction; 1 and 8 days after the injections). Healthy rats were locally administered with saline (FIGS. 4A, 4E, 4I, and 4M); Alveolar ridge-wounded rats were treated with saline by local administration (FIGS. 4B, 4F, 4J, 4N, and right on 4Q-S) or with exemplary PPA NPs (micellar particles) by local administration (FIGS. 4C, 4D, 4K, 4O, and left on 4Q-S) or IV administration (FIGS. 4D, 4G, 4L, and 4P) administrations. FIGS. 4Q-S present alveolar ridge-wounded rats treated with saline (right side rat) and with PPA NPs by local administration (left side rat).

FIGS. 5A-I present additional data obtained in the study illustrated in FIGS. 3A-B. FIGS. 5A-F are images of in-vivo full body analysis of the signal intensity of the exemplary labeled PPA NPs in untreated or locally injected (FIGS. 5A, 5C, and 5E), and in IV-injected (FIGS. 5B, 5D, and 5F) alveolar ridge-wounded rats. FIGS. 5A and 5B are in-vivo whole body images taken by black and white camera; FIGS. 5C and 5D are images of in-vivo whole body fluorescence detection; and FIGS. 5E and 5F are images of in-vivo whole body fluorescence intensity measurements, at day 10 of the experiment. FIGS. 5G and 5H are bar graphs showing the quantification of fluorescence following PPA NPs local (FIG. 5G) and IV (FIG. 5H) administration. The PPA NPs fluorescence signal intensity per unit area normalized to adjacent background intensity is expressed as fluorescence normalized units and presented as each group mean±SEM. FIG. 5I presents images of in-vivo full body analysis of the signal intensity of labeled PPA NPs in alveolar ridge-wounded rats, 3 days and 10 days following tail IV-injection.

FIGS. 6A-C present a schematic illustration of a study protocol (FIG. 6A), a photograph of an induction of an alveolar crest wound used in the study (FIG. 6B) and a schematic illustration of an exemplary PPA and the self-assembled enzyme-responsive fluorescent nanoparticles NPs formed therefrom and used in the study. (FIG. 6C). The presented exemplary PPA is a block copolymer comprised of block(s) of a polymer comprising backbone units having an MMP-peptide (purple) conjugated thereto (60%) and block(s) of a polymer comprising backbone units having an MMP-peptide (purple) and a Cy5.5 fluorescent dye (green) conjugated thereto (40%). The MMP-peptide comprises L-amino acids, and is represented also as L-PPA (MMP-responsive) or D-amino acids and is represented as D-PPA (MMP-non-responsive).

FIGS. 7A-L are in-vivo whole body and ex-vivo dissected maxilla fluorescence imaging following PPA-NPs administration. Black and white camera photographs (FIGS. 7A-C, and 7G-I) and fluorescence detection images (FIGS. 7D-F and 7J-L) of rats treated with L-PPA NPs (FIGS. 7A-F) and D-PPA NPs (FIGS. 7G-L) after wound induction.

FIGS. 7M-O present the data obtained in in-vivo whole body and ex-vivo dissected maxilla fluorescence imaging following PPA-NPs administration as presented in FIGS. 6A-C. FIGS. 7M-O are bar graphs showing the normalized fluorescence (FIGS. 7M-N) and right-to-left maxilla fluorescence ratio (FIG. 7O) as recorded in the study presented in FIGS. 6A-C. FIGS. 7M and 7N present in-vivo whole body (FIG. 7M) and ex-vivo dissected maxilla (FIG. 7N) imaging analyses of locally administered L-PPA and D-PPA accumulation in the inflamed tissue of wounded rats.

FIG. 7O presents the normalized fluorescence signal values obtained from (i) in-vivo whole body over time, following injecting the rats with L-PPA (red) or D-PPA (blue) (4 leftmost bar pairs); and (ii) ex-vivo isolated maxilla at day 29, in L-PPA- and D-PPA-injected rats (the rightmost bar pair). The fluorescence intensity was expressed as the fluorescence signal normalized to the exposure time and a 1000 pixel area, and is presented as each group mean±SEM.

FIG. 7P is black and white camera images and fluorescence detection images of rats that underwent enzyme responsive NPs administration, and no injection, at 4 and 8 days after the wound induction. In-vivo whole body and ex-vivo dissected maxilla fluorescence imaging and quantification following NPs tail vein administration.

FIG. 7Q presents ex-vivo dissected maxilla fluorescence quantification following tail vein NPs administration. The PPA fluorescence signal intensity per unit area normalized to adjacent background intensity is expressed as fluorescence normalized units and presented as each group mean±SEM.

FIGS. 8A-F present images of analyzed dissected maxilla coronal sections of control (healthy; FIGS. 8A, 8C, and 8E); and alveolar crest-wounded (FIGS. 8B, 8D, and 8F) rats, following the study presented in FIGS. 6A-C, as analyzed by histology (Haemotoxylin and Eosin) staining obtained by optical microscopy (FIGS. 8A and 8B); fluorescence microscopy (FIGS. 8C and 8D); and confocal microscopy (FIGS. 8E and 8F). In FIGS. 8A and 8B, the double headed arrows mark the extent of the oral mucosa in each micrograph. The different dissected sections are indicated in FIG. 8B and denote the (a) oral epithelium; (b) lamina propria connective tissue; and (c) maxillary bone. Scale bar is 500 μm.

FIG. 8G is a schematic presentation summarizing the study protocol and respective data of the study described in Example 2.

FIGS. 9A-E present exemplary synthetic schemes (FIGS. 9A, 9B and 9D) and respective analytical data (SEC-MALS; FIGS. 9C and 9D) of an exemplary dexamethasone-functionalized norbornene monomer NorDEX (FIG. 9A), of a ROMP polymerization thereof (FIG. 9B; analysis of the obtained polymer shown in FIG. 9C), and of a DEX-containing PPA prepared therefrom by ROMP co-polymerization with MMP-peptide-containing building blocks (FIG. 9D; analysis of the obtained co-polymer shown in FIG. 9E).

FIGS. 9D-G present an exemplary synthetic scheme (FIG. 9F) and respective analytical data (SEC-MALS; FIG. 9G) of a PEG12-containing DEX-Cy5.5-PPAs as an optional control PPA. SEC-MALS data show traces of PEG12 functionalized polymers (aqua and grey plots). Polymers without Cy5.5 (dark blue plot) were used for SEC-MALS measurement to avoid dye interference with light scattering.

FIGS. 10A-B present a schematic presentation of the syntheses of exemplary Dex-containing PPAs which further comprise a peptide attached to backbone units of the polymer, and their self-assembly into nanoparticles having a drug core and labeled peptide shell (FIG. 10A) and comparative SEC traces of the obtained PPAs (FIG. 10B). PPAs without Cy5.5 (NorDex₂₀; green) were used for characterization to avoid the dye interference with light scattering. The peptides are either L-MMP (SEQ ID NO: 1) in a PPA denoted PPA_{L} or NorMMP_{L}, D-MMP (SEQ ID NO: 2) in a PPA denoted PPA_{D} or NorMMP_{D}, or a random peptide (SEQ ID NO: 17) in a PPA denoted PPA_{R} or NorMMP_{R}.

FIG. 10C presents comparative HPLC plots, showing MMP-9 cleavage of NorMMP_{R}NorMMP_{D} or NorMMP_{L}), and a control cleavable peptide sequence LAGGWGERDGS (SEQ ID NO: 18) upon 24 hours of incubation with MMP-9 at 37° C., showing that NorMMP_{L} was completely cleaved, while the NorMMP_{R} and NorMMP_{D} remained substantially intact. The arrows indicate the change in HPLC traces before and after MMP-9 treatment.

FIGS. 11A-C present TEM images showing micellar nanoparticles (NPs) formed of DEX-containing L-MMP-containing PPA (PPA_{L}; denoted as L-MMP NPs or NP_{L}; FIG. 11A), D-PPA (PPA_{D}; denoted as D-MMP NPs or NP_{D} FIG. 11B), and R-Pep (PPA_{R}; denoted as R-MMP NPs or NP_{R}; FIG. 11C).

FIG. 11D presents TEM images of nanoparticles before and after thermolysin treatment. Scale bar is 100 μm.

FIGS. 12A-D present TEM images showing L-PPA NPs (NP_{L}; FIGS. 12A and 12B) and D-PPA NPs (NP_{D}; FIGS. 12C and 12D), in the absence (FIGS. 12A and 12C) and presence (FIGS. 12B and 12D) of a MMP enzyme, showing the formulation of polymeric aggregates following contact with the enzyme. Scale bar is 100 nm.

FIGS. 13A-B present comparative plots (FIG. 13A) and a bar graph (FIG. 13B), showing blood glucose level before (day 0) and after (days 7, 14) induction of diabetes in rats. In FIG. 13B, the dashed black line marks the value of 270 mg/dL above which the animals are considered hyperglycemic.

FIGS. 14A-G are photographs of a collagen membrane implantation under the scalp of a diabetic rat, a model procedure for inducing chronic inflammation while overexpressing MMPs. The blue arrow in FIG. 14G marks the collagen membrane.

FIGS. 15A-F present in-vivo black and white camera photographs and fluorescence imaging of diabetic rats following collagen membrane implantation (FIGS. 15A, 15B, FIGS. 15D-F) or un-injured (FIG. 15B). FIG. 15A presents diabetic rat treated with D-PPA and FIGS. 15B-C present diabetic rats treated with L-PPA. FIG. 15D is a black and white camera photograph; FIG. 15E is in-vivo fluorescence imaging; and FIGS. 15A-C and 15F are an overlay of black and white and fluorescence imaging.

FIG. 16A-C are bar graphs showing the normalized fluorescence detected in-vivo (FIGS. 16A and 16B) and ex-vivo (FIG. 16C) in diabetic rats after collagen membrane implantation and a following treatment with NP_{D} (D-PPA), NPL (L-PPA), and NP_{R} (R-PPA).

FIGS. 17A-C present histological assessment data of residual collagen membrane content, analyzed by histological sections of HRP-Avidin stained calvaria from control, and dexamethasone- or NP_{L} (denoted as PPA-dexa)-treated rats (FIG. 17A; the collagen content of the tissue appears in brown, in between the periosteum and the calvaria bone); and segmentation-based quantification of the collagen content percentage of the membrane in each of these study groups (FIGS. 17B and 17C).

FIGS. 18A and 18B are schematic illustrations of additional study protocols in accordance with the protocols described in Example 2 (FIG. 18A) and the study protocol as described in Example 3 (FIG. 18B).

### DESCRIPTION OF SPECIFIC EMBODIMENTS OF THE INVENTION

The present invention, in some embodiments thereof, relates to therapy, and more particularly, but not exclusively, to novel block copolymers that comprise an anti-inflammatory agent-containing and a stimuli-responsive peptide-containing polymer amphiphilic materials (PPAs) and to micellar particles formed thereof which are usable, for example, in the treatment of inflammation.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Inflammation is a body's natural response to injury or infection. Currently available therapies to treat local and systemic inflammation, such as systemic small molecule antibiotics, suffer from fast clearance (within days) and poor localization at the diseased site, leading to side effects and decreased efficacy.

The targeted delivery of enzyme-responsive nanoparticles to specific tissues can be a valuable, minimally invasive approach for drug delivery applications. Different drug administration modes to the inflamed tissues may be evaluated, and the most efficient in-vivo and ex-vivo methods to assess the accumulation of the NPs in the evaluated tissues may be determined.

Chronic inflammation involves constant or repetitive activation of the immune system, and can lead to tissue damage and organ dysfunction. In addition to the limitations associated with currently available therapies for inflammation in general, discussed hereinabove, currently available systemic medications for treating diseases or disorders associated with chronic inflammation typically require repeated dosing due to limited drug circulation half-lives and poor tissue penetration. This commonly leads to patient incompliance and can increase the risk of dose-dependent off-target effects.

An exemplary condition (disease or disorder) that is associated with chronic inflammation is diabetes. Diabetes has a major impact on the patients' health and their risk of developing severe complications. These complications include increased susceptibility to delayed wound healing, and deterioration of inflammatory and infection-related pathologies. A common manifestation of most of the diabetic complications is an exaggerated and prolonged inflammatory response, including excessive production of pro-inflammatory cytokines together with increased numbers of immune cells. The increased presence of inflammation, as evidenced either by its cellular makeup or by its molecular repertoire, has been described in many extra-oral, and oral tissues of diabetic humans or animals. Many anti-diabetes drugs possess anti-inflammatory properties.

Diabetes is accompanied by numerous complications, among which are impaired wound healing and increased risk of infection that may lead to large incidence of secondary infections and resistance to standard treatments. Thus, there is a need to develop efficient methods to target drugs delivery to the affected tissues.

Inflammation is also one of the major factors in dental pathology, which leads to periodontal diseases, and massive loss of bone [Ebersole et al., Periodontol 2000, 2016, 72, 54-75].

The present inventors have conceived using the recently emerging stimuli-responsive or “smart” materials that respond to disease-associated triggers for designing targeted and sustained drug delivery systems for treating inflammation, to thereby circumvent the limitations associated with currently available therapies. The present inventors have conceived that using such systems is particularly beneficial for treating chronic inflammation.

More specifically the present inventors have conceived using, as an exemplary model, enzyme-responsive PPA nanoparticles, which can target inflamed tissues through the enhanced permeability and retention (EPR) effect and aggregate in response to upregulated inflammatory enzyme such as an MMP enzyme. Thus, targeted delivery of different anti-inflammatory drugs, by means of PPA NPs may improve treatment efficacy in inflammatory lesions.

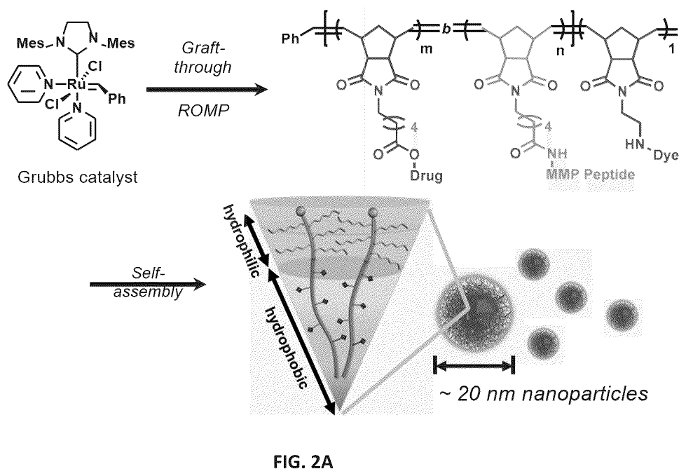

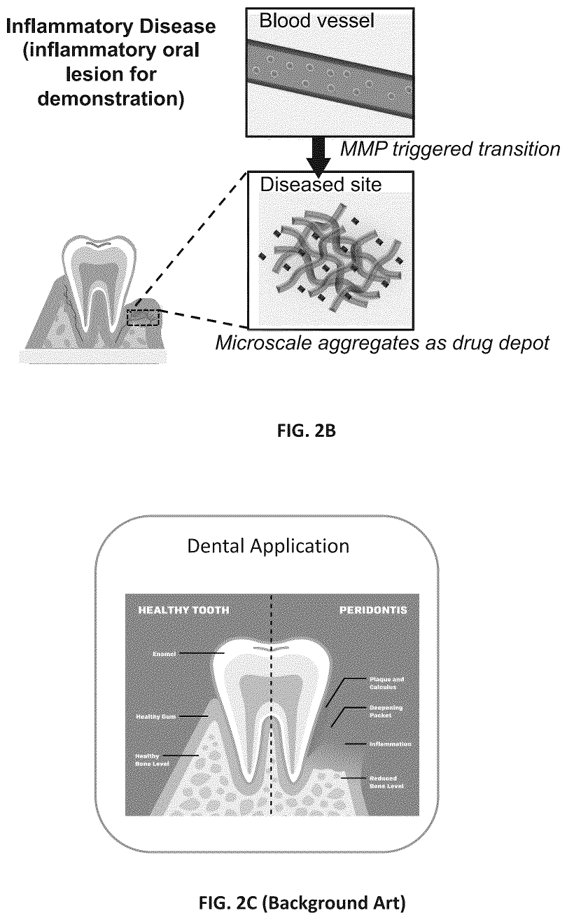

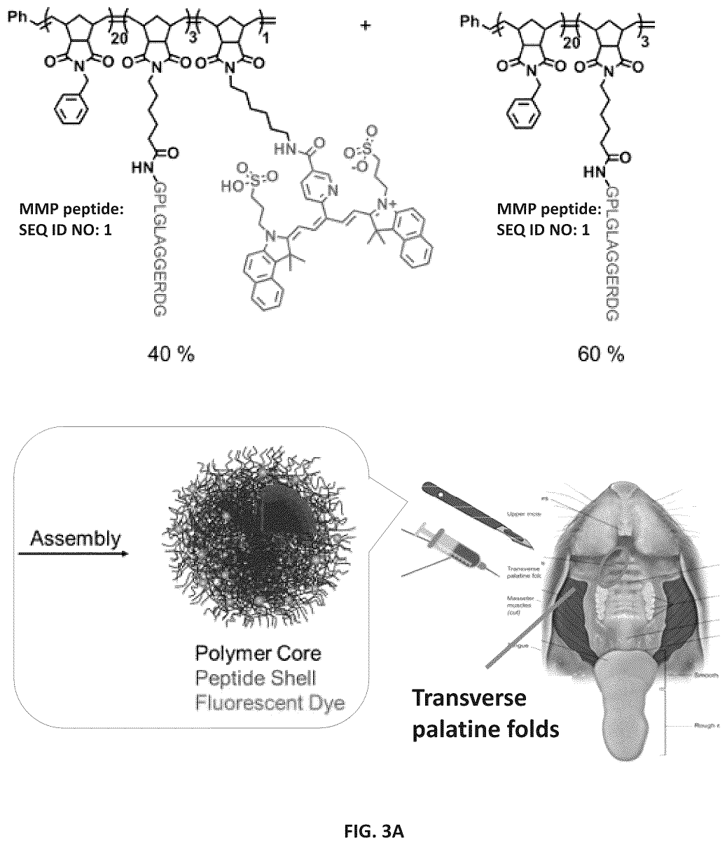

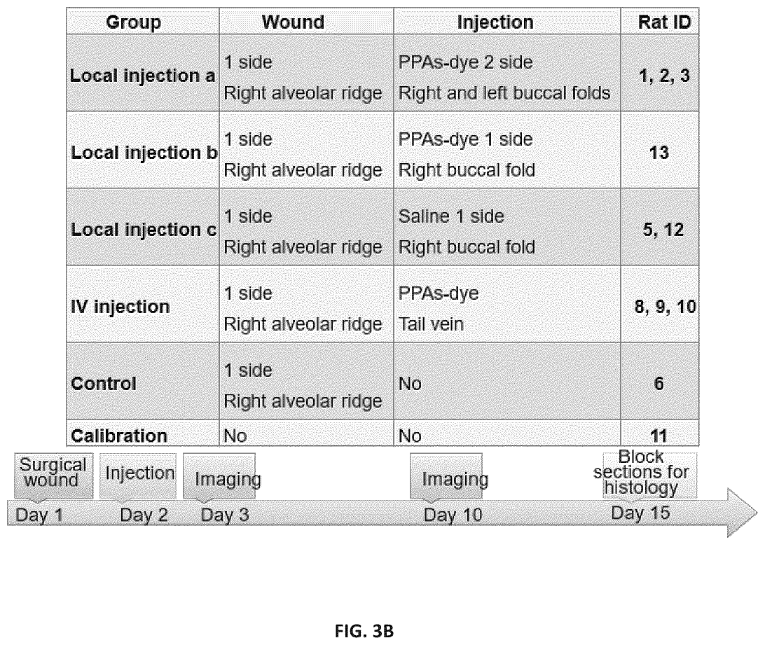

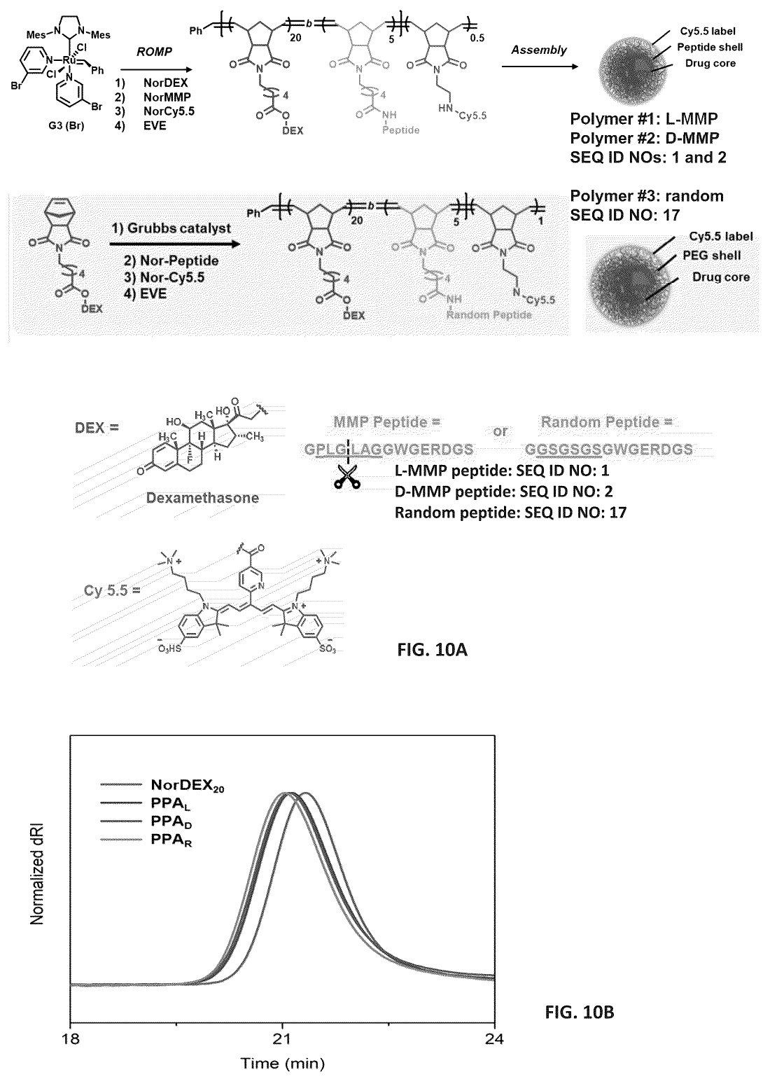

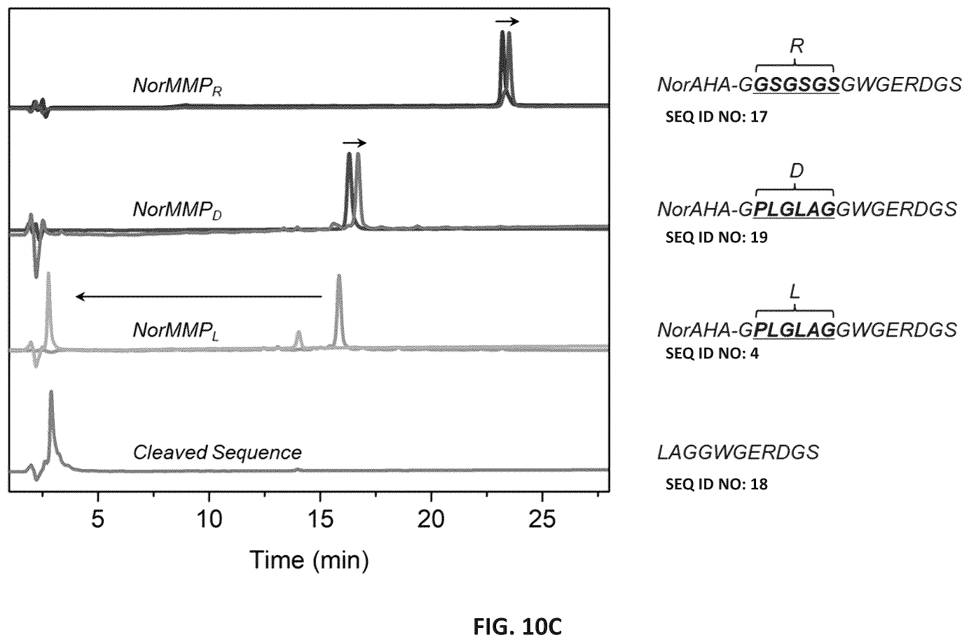

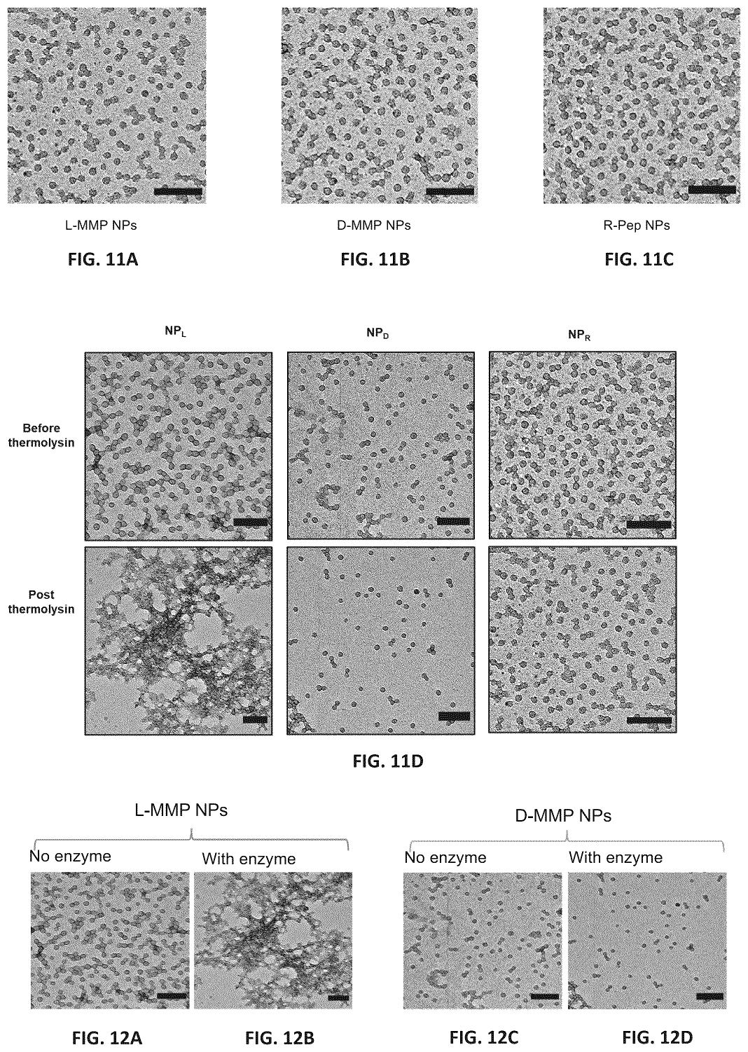

Thus, polymer peptide amphiphiles (PPAs) were designed as an enzyme responsive “smart” drug delivery platform to treat inflammation-associated diseases. Well-defined PPAs were prepared via living and controlled ring-opening metathesis polymerization (ROMP) using functionalized ROMP monomers such as norbornene monomers, carrying a matrix metalloproteinase (MMP, a family of upregulated inflammatory enzyme)-cleavable peptide, an anti-inflammatory drug such as the FDA approved dexamethasone and/or a fluorescent labeling agent such as Cy5.5. These PPAs self-assembled into micellar nanoparticles (about 20 nm in diameter) with MMP peptide shell and drug core in buffer solution. See, for example, FIGS. 2A and 10A. In the presence of MMPs, the peptide shell was cleaved, triggering a morphological transition into micro-scale aggregates, which serve as a depot for sustained drug release through enzymolysis and hydrolysis. See, for example, FIGS. 10C, 11A-D and 12A-D.

The peptide polymer amphiphiles (PPAs) were developed for on-demand delivery of therapeutics, such as anti-inflammatory agents, to the inflamed tissues.

The PPAs were demonstrated to be modularly synthesized via ring-opening metathesis polymerization (ROMP) using functionalized monomers and self-assemble into micellar nanoparticles (PPA NPs) in buffer solution. The small size enables the material to reach the diseased tissue through the leaky blood vessels induced by inflammation (via the EPR effect) and preferentially accumulate there as a drug depot post the MMP-triggered aggregation.

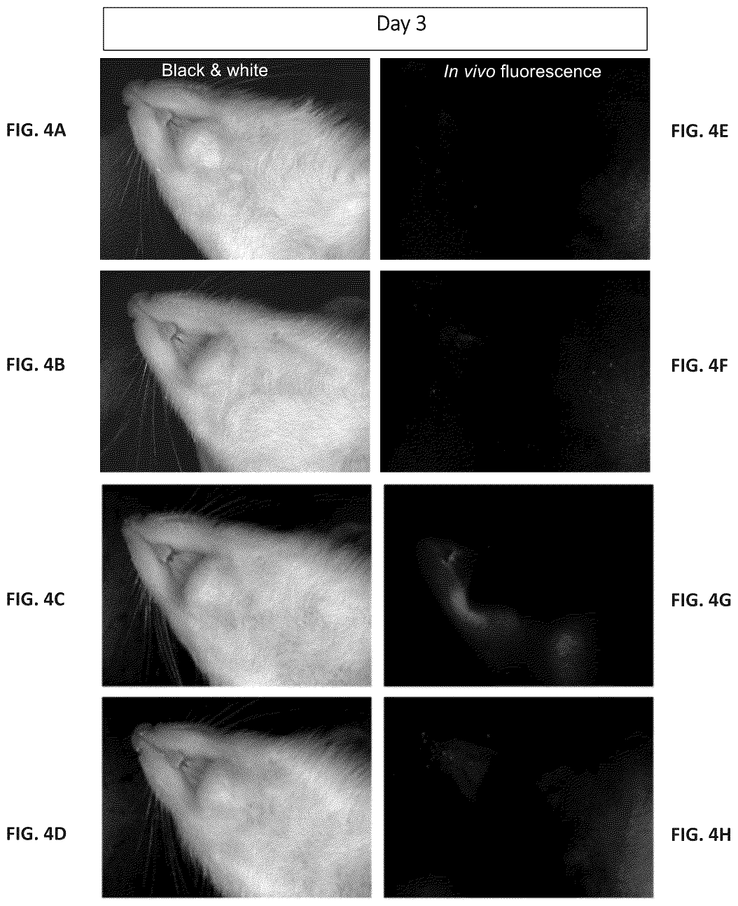

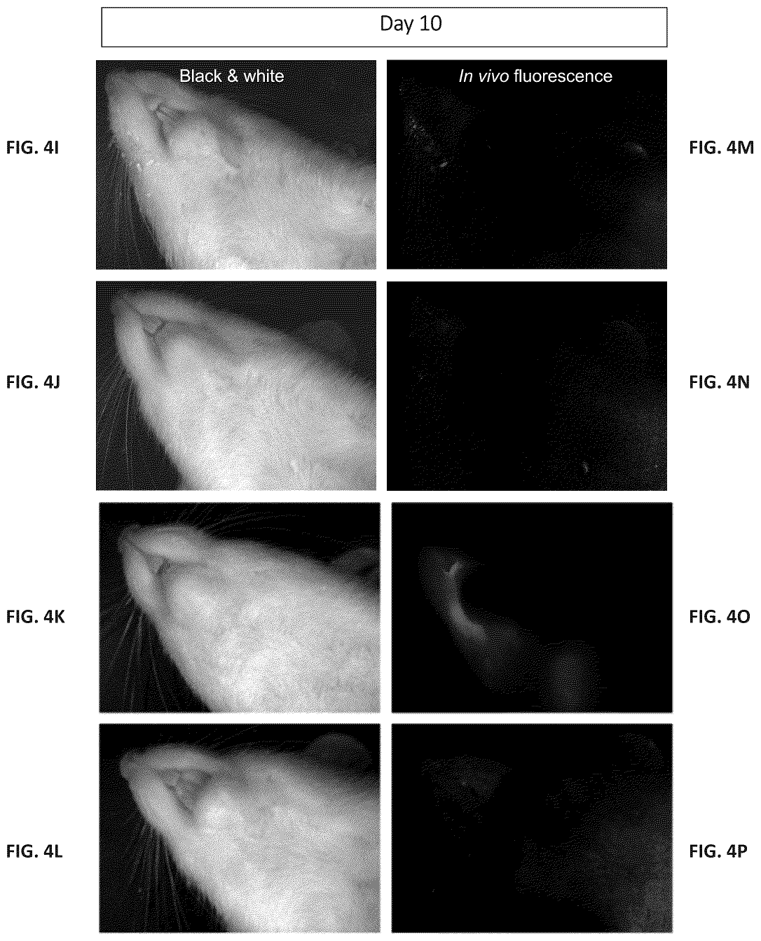

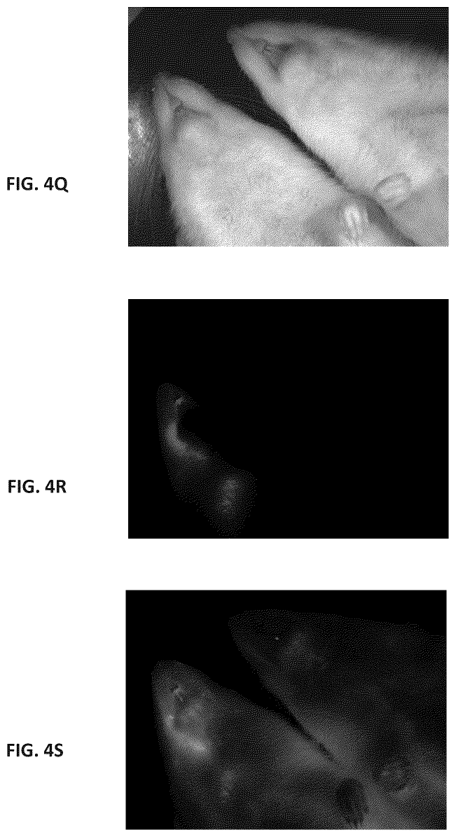

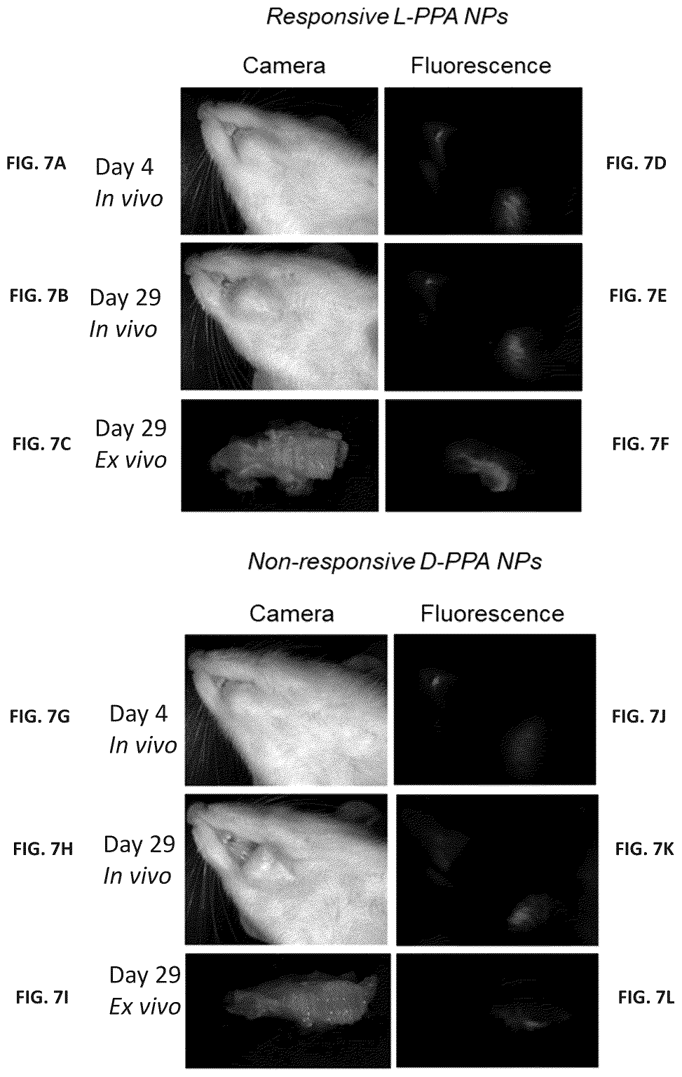

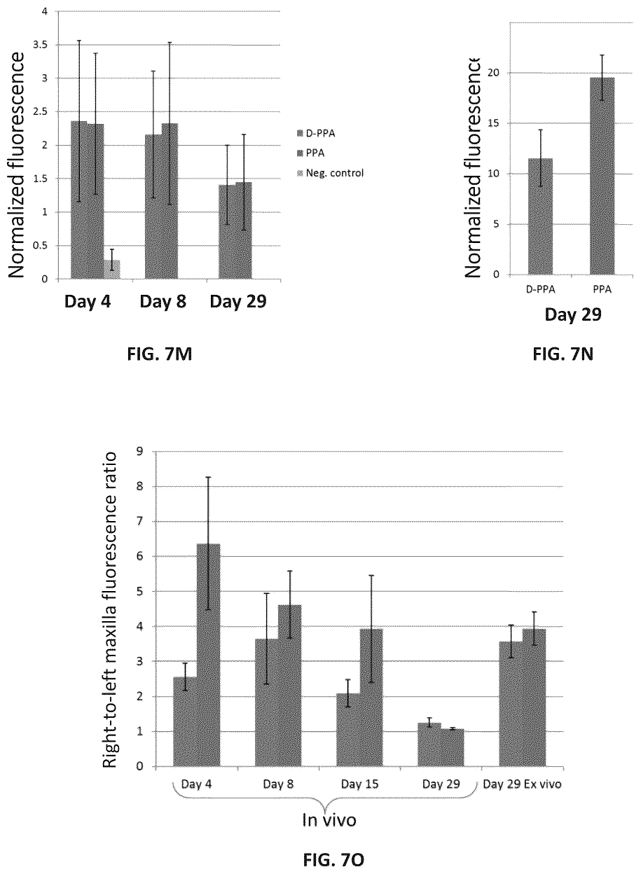

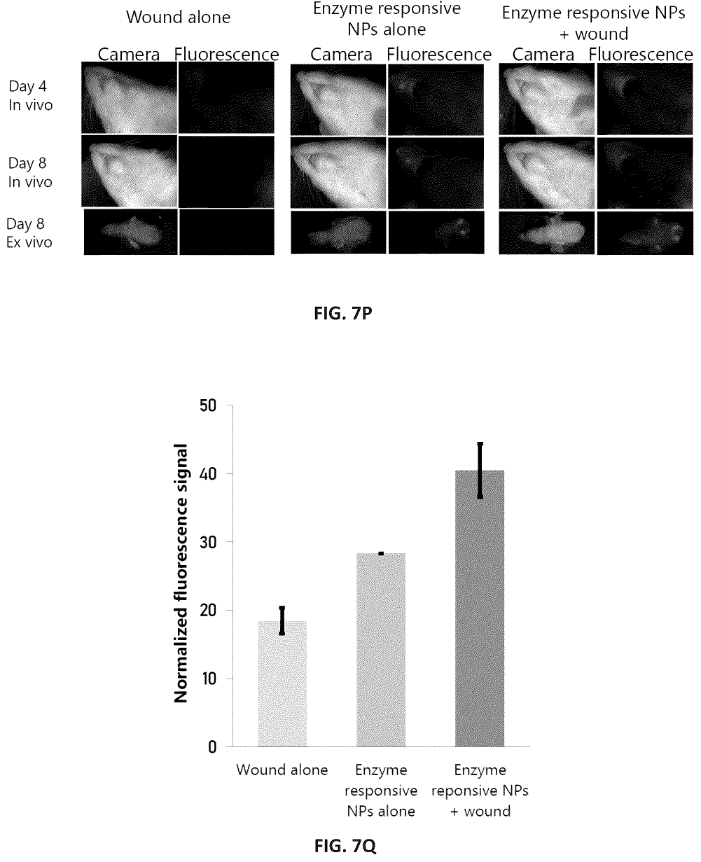

The effect of the designed PPA NPs in the treatment of inflammatory oral pathologies using established rat animal models to assess the accumulation and therapeutic effects of enzyme-responsive drug-binding nanoparticles in the inflamed oral tissues was studied. See, for example, FIGS. 4A-S. In a rat periodontal flap model, PPA localization and prolonged retention (up to 30 days post injection) in the diseased oral tissue following both local and intravenous administration were observed. See, for example, FIGS. 7A-F and 4P. Different drug administration modes to the inflamed tissues were evaluated, as well as in-vivo and ex-vivo methods to assess the accumulation of the NPs in the evaluated tissues.

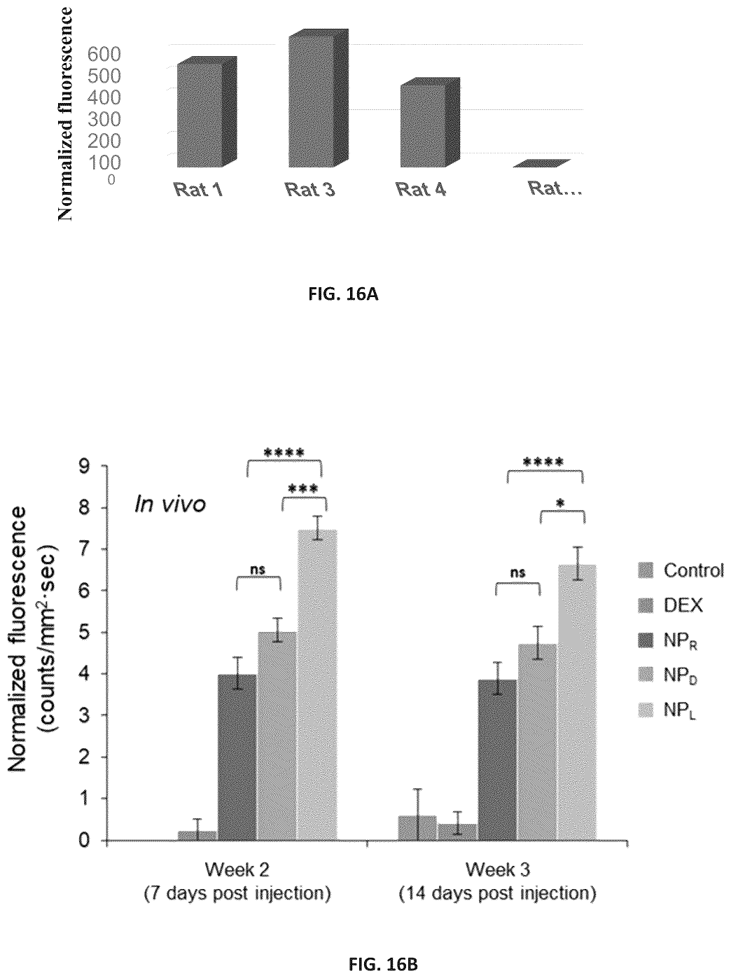

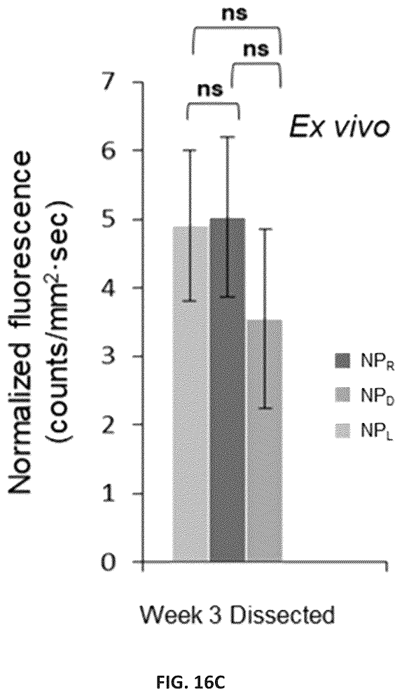

The anti-inflammatory effect of the drug-loaded PPA NPs has also been evaluated through a diabetic rate model, in which systemic inflammation is induced. A collagen membrane that can be degraded by the inflammatory enzymes was embedded under the scalp of these diabetic rats. The histology, volume, and mass of the membrane, as well as the quantity and activity of the inflammatory markers, post PPA NPs treatment were assessed for determining the efficacy. See, for example, FIGS. 16B-C and 17A-C.

The obtained data demonstrate that incorporation of an anti-inflammatory drug such as dexamethasone into the PPAs improves its therapeutic efficacy of reducing inflammation, potentially by increasing local drug concentration and prolonging its circulation half-life, thereby demonstrating the potential use of PPA NPs as a targeted drug delivery methodology for the treatment of diabetic and other (e.g., chronic) inflammatory conditions.

The newly designed PPA NPs represent a generalizable approach for targeted and on demand delivery of various drugs, including small molecules, peptide-based and oligonucleotide-based therapeutics, to treat inflammatory diseases, which can reduce drug off-target effects and frequency of dosing, while improving efficacy and tolerated dosage.

The novel PPAs were synthesized in a modular manner and are compatible with various therapeutics, ranging from small molecule drugs to peptide/oligosaccharide-based therapeutics. It can potentially reduce the off-target side effects of the parent drug and at the same time increase efficacy.

Embodiments of the present invention relate to newly designed PPAs, which are also referred to herein as block copolymers featuring one or more hydrophilic block(s) and one or more hydrophobic (e.g., drug-containing) blocks; to newly designed PPA NPs formed therefrom, which are also referred to herein as micellar particles (e.g., micellar nanoparticles), and to uses thereof in the treatment of inflammation, and of chronic inflammation in particular.

According to an aspect of some embodiments of the present invention, there is provided a block copolymer that comprises at least one first block composed of a first plurality of backbone units covalently linked to one another and at least one second block of a second plurality of backbone units covalently linked to one another.

According to some of any of the embodiments described herein, the first block comprises at least one anti-inflammatory agent covalently attached, directly or via a linking moiety or group, to at least a portion of the first plurality of backbone units composing the first block; and
the second block comprises at least one hydrophilic moiety covalently attached to at least a portion of the second plurality of backbone units composing the second block.

According to some of any of the embodiments described herein, the hydrophilic moiety comprises an amino acid sequence that is capable of interacting with an inflammatory biomarker, and is also referred to herein as inflammatory biomarker-responsive peptide moiety.

According to some of any of the embodiments described herein, the block copolymers are capable of forming a micellar particle, preferably a micellar nanoparticle, when contacting a suitable solvent, preferably an aqueous solution (e.g., a buffer). Thus, the block copolymers are arranged such that upon contacting the suitable solvent, the formed micellar particles feature a hydrophobic core and a hydrophilic shell that comprises the inflammatory biomarker-responsive peptide moiety. The hydrophobic core comprises the anti-inflammatory agent. The block copolymer is therefore designed so as to include a hydrophobic anti-inflammatory agent, and/or to include other moieties or groups that promote the formation of a core that comprises an anti-inflammatory agent.

Block copolymers that are capable of forming peptide-containing micellar particles as described herein are also referred to herein as PPAs.

According to an aspect of some embodiments of the present invention, there is provided a micellar particle that comprises a plurality of the block copolymers as described herein in any of the respective embodiments and any combination thereof, which is arranges such that it comprises a hydrophobic core that comprises the anti-inflammatory agent and a hydrophilic shell that comprises the inflammatory biomarker-responsive peptide moiety. The micellar particle is preferably a micellar nanoparticle, and is also referred to herein as PPA NP.

According to an aspect of some embodiments of the present invention, there is provided a micellar particle (PPA NP) as described herein in any of the respective embodiments and any combination thereof, for use in the treatment of inflammation, and in particular chronic inflammation, as described herein.

According to an aspect of some embodiments of the present invention, there is provided a block copolymer (PPA) as described herein in any of the respective embodiments and any combination thereof, for use in the treatment of inflammation, and in particular chronic inflammation, as described herein, by contacting the block copolymer with a suitable solution for forming a micellar particle as described herein which is usable for treating the inflammation.

According to an aspect of some embodiments of the present invention, there is provided a block copolymer as described herein in any of the respective embodiments for use in the preparation of a micellar particle as described herein in any of the respective embodiments, which is usable in the treatment of inflammation as described herein.

According to an aspect of some embodiments of the present invention, there is provided a use of block copolymer as described herein in any of the respective embodiments in the preparation of a micellar particle as described herein in any of the respective embodiments, which is usable in the treatment of inflammation as described herein.

According to an aspect of some embodiments of the present invention, there is provided block copolymer as described herein in any of the respective embodiments in the manufacture of a medicament for treating inflammation as described herein, wherein the block copolymer is used for preparing a micellar particle as described herein in any of the respective embodiments, which is usable in the treatment of inflammation as described herein.

According to an aspect of some embodiments of the present invention, there is provided a method of treating inflammation as described herein, which comprises administering to a subject in need thereof a micellar particle as described herein in any of the respective embodiments and any combination thereof.

According to an aspect of some embodiments of the present invention, there is provided a method of treating inflammation as described herein, which comprises contacting a block copolymer (PPA) as described herein in any of the respective embodiments and any combination thereof, so thereby form a micellar particle as described herein, and administering to a subject in need thereof a micellar particle as described herein in any of the respective embodiments and any combination thereof.

According to an aspect of some embodiments of the present invention, there is provided a use of a micellar particle as described herein in the manufacture of a medicament for treating inflammation as described herein.

According to an aspect of some embodiments of the present invention, there is provided a method of forming a polymeric aggregate in an inflamed tissue, the method comprising contacting the micellar particle as described herein in any of the respective embodiments and any combination thereof with the inflamed tissue. The polymeric aggregate is formed as a result of the interaction of the hydrophilic moiety with the inflammatory biomarker that is upregulated in the inflamed tissue.

The contacting can be effected in vivo or ex-vivo or in vitro.

According to an aspect of some embodiments of the present invention, there is provided a micellar particle as described herein for use in forming a polymeric aggregate in an inflamed tissue upon contacting the micellar particle with the inflamed tissue.

According to an aspect of some embodiments of the present invention, there is provided a block copolymer (PPA) as described herein for use in forming a polymeric aggregate in an inflamed tissue upon contacting the block copolymer with a suitable solution to thereby form a micellar particle and contacting the micellar particle with the inflamed tissue, as described herein.

According to an aspect of some embodiments of the present invention, there is provided a method of forming a polymeric aggregate in an inflamed tissue, the method comprising contacting the block copolymer with a suitable solution to thereby form a micellar particle and contacting the micellar particle with the inflamed tissue, as described herein.

According to an aspect of some embodiments of the present invention there is provided a polymeric aggregate, formed upon contacting a micellar particle as described herein with an inflamed tissue.

According to an aspect of some embodiments of the present invention there is provided a polymeric aggregate, formed upon contacting a block copolymer or a plurality thereof with a suitable solvent to thereby form a micellar particle as described herein and contacting a micellar particle as described herein with an inflamed tissue.

According to an aspect of some embodiments of the present invention there is provided a kit that comprises a micellar particle or a plurality thereof as described herein or a pharmaceutical composition comprising same and means to administer the particle or the pharmaceutical composition to a subject afflicted by inflammation as described herein.

According to an aspect of some embodiments of the present invention there is provided a kit that comprises a micellar particle as described herein or a pharmaceutical composition comprising same and means to administer the particle or the pharmaceutical composition to a subject afflicted by inflammation as described herein.

According to an aspect of some embodiments of the present invention there is provided a kit that comprises a plurality of block copolymers (PPAs) as described herein in any of the respective embodiments, a solvent suitable for contacting the block copolymers to thereby form micellar particles comprising same as described herein in any of the respective embodiments, means to administer the micellar particles to a subject afflicted by inflammation as described herein. Optionally, the kit further comprises a pharmaceutical acceptable carrier (e.g., an aqueous solution) for forming a pharmaceutical composition and means to administer the pharmaceutical composition to a subject afflicted by inflammation as described herein.

According to some of any of the embodiments described herein, any of the kits as described herein are identified for use in treating inflammation as described herein.

Injecting or locally administering the particles or compositions can include syringes and/or needles, suitable for delivering the particles, and/or systems or devices for guided local administration, and/or any other means known in the art.

According to some of any of the embodiments described herein, treating inflammation is by administering the micellar particles as described herein or a pharmaceutical composition comprising same, as described herein, locally, to the inflamed tissue or to a nearby vicinity.

Alternatively, the micellar particles of the pharmaceutical composition is administered systemically, for example, intravenously, intramuscularly, orally, or via any other mode of administration as described herein.

According to an aspect of some embodiments of the present invention there is provided a method of treating inflammation in a subject in need thereof, as described herein, which comprises locally administering to an inflamed tissue of the subject, or to a nearby vicinity thereof, a therapeutically effective amount of the micellar particle of a composition comprising same.

According to an aspect of some embodiments of the present invention there is provided a micellar particle of a pharmaceutical composition comprising same, for use in treating inflammation in a subject in need thereof, as described herein, wherein the treatment comprises, or is effected by, locally administering to an inflamed tissue of the subject, or to a nearby vicinity thereof, a therapeutically effective amount of the micellar particle of a composition comprising same.

Without being bound by any particular theory, it is assumed that while inflammatory conditions are identified at the core of many different diseases (e.g. chronic diabetes), anti-inflammatory treatments through efficient and safe drug delivery are desirable. However, there may be systemic side effects that are associated with the application of different anti-inflammatory drugs (such as steroidal and non-steroidal anti-inflammatory drugs), especially when these are given systemically or orally. Therefore, the reduction of these side effects by local and targeted drug delivery could have a significant effect on the reduction of these possible complications.

Localized and targeted drug delivery are alternative forms of drug administration due to their ability to avoid off-target side effects. A drug that is delivered locally at the target tissue reaches therapeutic concentrations only where it is injected. However, the effectiveness of the locally administrated drug may be adversely affected by the local inflammation at the diseased application site. Targeted delivery circulates throughout the entire body, but is protected from exhibiting therapeutic effects in locations that are not desired, resulting in the same off-target protective effects. However, when the targeted drug delivery is through I.V. or I.M. injections it may be adversely affected by deficient blood perfusion associated with the patient disease condition (such as in diabetic patients).

Therefore, local injection of a targeted drug near a site of interest, namely in a close vicinity to an inflamed tissue (e.g., a diabetic wound), should result in the same off-target protective effects and at the same time avoid adverse effects such as deficient blood perfusion.

According to an aspect of some embodiments of the present invention, there is provided a method of preparing the micellar particle as described herein in any of the respective embodiments and any combination thereof, which comprises contacting the plurality of block copolymers with a suitable solvent that promotes micelle formulation, for example, an aqueous solution.

### Treatment of Inflammation:

Inflammatory diseases and disorders generally encompass diseases and disorders associated with inflammation.

Herein, the phrase “treating inflammation” encompasses treating an inflammatory disease or disorder as described herein.

The term “inflammation” as used herein refers to the general term for local accumulation of fluids, plasma proteins, and white blood cells initiated by physical injury, infection, or a local immune response. Inflammation may be associated with several signs e.g. redness, pain, heat, swelling and/or loss of function. Inflammation is an aspect of many diseases and disorders, including but not limited to diseases related to immune disorders, viral and bacterial infection, arthritis, autoimmune diseases, collagen diseases, allergy, asthma, pollinosis, and atopy (as described in further detail below).

Thus, inflammation can be triggered by injury, for example injury to skin, muscle, tendons, or nerves. Inflammation can be triggered as part of an immune response, e.g., pathologic autoimmune response. Inflammation can also be triggered by infection, where pathogen recognition and tissue damage can initiate an inflammatory response at the site of infection.

Inflammation according to the present teachings may be associated with chronic (long term) inflammatory diseases or disorders or acute (short term) inflammatory diseases or disorders.

According to a specific embodiment, inflammation according to the present teachings is chronic inflammation, associated with chronic (long-term) inflammatory diseases or disorders.

According to a specific embodiments, inflammation, e.g., chronic inflammation, is associated with diabetes, for example, type-I diabetes.

According to an aspect of some embodiments of the present invention, there is provided a method of treating diabetes, which comprises administering to a diabetic subject a therapeutically effective amount of a micellar particle as described herein or a pharmaceutical composition comprising same.

According to some embodiments of the present invention, any of the methods and uses as described herein for a micellar particle or a pharmaceutical composition comprising same, or a block copolymer, are for treating diabetes as described herein.

According to some embodiments, the inflammation is associated with a disease selected from the group consisting of an infectious disease, an autoimmune disease, a hypersensitivity associated inflammation, a graft rejection and an injury.

According to some embodiments, the inflammation comprises a skin inflammation.

Diseases characterized by inflammation of the skin, include but are not limited to dermatitis, atopic dermatitis (eczema, atopy), contact dermatitis, dermatitis herpetiformis, generalized exfoliative dermatitis, seborrheic dermatitis, drug rashes, erythema multiforme, erythema nodosum, granuloma annulare, poison ivy, poison oak, toxic epidermal necrolysis, roseacae, psoriasis and acne. Inflammation can also result from physical injury to the skin.

Inflammation may be triggered by various kinds of injuries to muscles, tendons or nerves. Thus, for example, inflammation may be caused by repetitive movement of a part of the body i.e. repetitive strain injury (RSI). Diseases characterized by inflammation triggered by RSI include, but are not limited to, bursitis, carpal tunnel syndrome, Dupuytren's contracture, epicondylitis (e.g. tennis elbow), ganglion (i.e. inflammation in a cyst that has formed in a tendon sheath, usually occurring on the wrist), rotator cuff syndrome, tendinitis (e.g., inflammation of the Achilles tendon), tenosynovitis, and trigger finger (inflammation of the tendon sheaths of fingers or thumb accompanied by tendon swelling).

Many diseases related to infectious diseases include inflammatory responses, where the inflammatory responses are typically part of the innate immune system triggered by the invading pathogen. Inflammation can also be triggered by physical (mechanical) injury to cells and tissues resulting from the infection. Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases. According to one embodiment, examples of infections characterized by inflammation include, but are not limited to, encephalitis; meningitis; encephalomyelitis; viral gastroenteritis; viral hepatitis.

Furthermore, many immune disorders include acute or chronic inflammation. For example, arthritis is considered an immune disorder characterized by inflammation of joints, but arthritis is likewise considered an inflammatory disorder characterized by immune attack on joint tissues.

Inflammation according to the present teachings may be associated with a deficient immune response (e.g., HIV, AIDS) or with an overactive immune response (e.g., allergy, autoimmune disorders). Thus, inflammation according to the present teachings may be associated with any of the following:

Inflammatory Diseases Associated with Hypersensitivity:

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like β-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tb1 lymphocyte mediated hypersensitivity and Tn2 lymphocyte mediated hypersensitivity.

According to a specific embodiment, the ocular disease is age-related macular degeneration (AMD).

According to a specific embodiment, the age-related macular degeneration (AMD) is atrophic, non-neovascular (aAMD).

According to a specific embodiment, the age-related macular degeneration (AMD) is neovascular.

### Autoimmune Diseases:

Autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost.2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome, diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Siogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

According to one embodiment, the autoimmune disease is Crohn's disease, psoriasis, scleroderma or rheumatoid arthritis.

The autoimmune diseases as described herein are considered as associated with chronic inflammation.

### Graft Rejection Diseases:

Examples of diseases associated with transplantation of a graft include, but are not limited to, graft rejection, chronic graft rejection, subacute graft rejection, hyperacute graft rejection, acute graft rejection and graft versus host disease.

### Allergic Diseases:

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

The allergic diseases as described herein are considered as associated with chronic inflammation.

### Oral Inflammatory Diseases:

According to some of any of the embodiments described herein, the inflammation is in an oral cavity. Examples include periodontitis, Gingivitis and pulpitis

According to some of the any of the embodiments described herein, the inflammation is a chronic inflammation, associated with a disease or disorder such as a chronic oral inflammation, an autoimmune disease (e.g., diabetes, arthritis and the like, as described herein), a chronic infection, and/or an allergic disease as described herein.

### Anti-Inflammatory Agent:

Herein, an anti-inflammatory agent is also referred to as an anti-inflammatory drug or simply as a drug or a therapeutic or a therapeutically active agent.

Exemplary anti-inflammatory agents or drugs that are usable in the context of the present embodiments include, but are not limited to, alclofenac; alclometasone (e.g., alclometasone dipropionate); algestone (e.g., algestone acetonide); alpha amylase; amcinafal; amcinafide; amfenac (e.g., amfenac sodium); amiprilose (e.g., amiprilose hydrochloride); anakinra; anirolac; anitrazafen; apazone; aspirin; balsalazide disodium; bendazac; benoxaprofen; benzydamine (e.g., benzydamine hydrochloride); bromelains; broperamole; budesonide; carprofen; cicloprofen; cintazone; cliprofen; clobetasol (e.g., clobetasol propionate, clobetasone butyrate); clopirac; cloticasone (cloticasome propionate); cormethasone (cormethasone acetate); cortodoxone; deflazacort; desonide; desoximetasone; dexamethasone (e.g., dexamethasone dipropionate); diclofenac (e.g., diclofenac potassium, diclofenac sodium); diflorasone (e.g., diflorasone diacetate); diflumidone (e.g., diflumidone sodium); diflunisal; difluprednate; diftalone; drocinonide; endrysone; enlimomab; enolicam (e.g., enolicam sodium); epirizole; etodolac; etofenamate; felbinac; fenamole; fenbufen; fenclofenac; fenclorac; fendosal; fenpipalone; fentiazac; flazalone; fluazacort; flufenamic acid; flumizole; flunisolide (e.g., flunisolide acetate); flunixin (e.g., flunixin meglumine); fluocortin (e.g., fluorcortin butyl); fluorometholone (e.g., fluorometholone acetate); fluquazone; flurbiprofen; fluretofen; fluticasone (e.g., fluticasone propionate); furaprofen; furobufen; halcinonide; halobetasol (e.g., halobetasol propionate); halopredone (e.g., halopredone acetate); ibufenac; ibuprofen (e.g., ibuprofen aluminum, ibuprofen piconol); ilonidap; indomethacin (e.g., indomethacin sodium); indoprofen; indoxole; intrazole; isoflupredone (e.g., isoflupredone acetate); isoxepac; isoxicam; ketoprofen; lofemizole (e.g., lofemizole hydrochloride); lomoxicam; loteprednol (e.g., loteprednol etabonate); meclofenamate (e.g., meclofenamate sodium, meclofenamic acid); meclorisone (e.g., meclorisone dibutyrate); mefenamic acid; mesalamine; meseclazone; methylprednisolone (e.g., methylprednisolone suleptanate); momiflumate; nabumetone; naproxen (e.g., naproxen sodium); naproxol; nimazone; olsalazine (e.g., olsalazine sodium); orgotein; orpanoxin; oxaprozin; oxyphenbutazone; paranyline (e.g., paranyline hydrochloride); pentosan polysulfate (e.g., pentosan polysulfate sodium); phenbutazone (e.g., phenbutazone sodium glycerate); pirfenidone; piroxicam (e.g., piroxicam cinnamate, piroxicam olamine); pirprofen; prednazate; prifelone; prodolic acid; proquazone; proxazole (e.g., proxazole citrate); rimexolone; romazarit; salcolex; salicylate (e.g., salicylic acid); salnacedin; salsalate; sanguinarium (e.g., sanguinarium chloride); seclazone; sermetacin; sudoxicam; sulindac; suprofen; talmetacin; talniflumate; talosalate; tebufelone; tenidap (e.g., tenidap sodium); tenoxicam; tesicam; tesimide; tetrydamine; tiopinac; tixocortol (e.g., tixocortol pivalate); tolmetin (e.g., tolmetin sodium); triclonide; triflumidate; zidometacin; and zomepirac (e.g., zomepirac sodium).

An anti-inflammatory agent can be a steroidal or non-steroidal agent.

Alternatively, or in addition, the anti-inflammatory agent is an anti-inflammatory peptide, or an anti-inflammatory oligonucleotide. Any anti-inflammatory peptides and/or oligonucleotides are contemplated.

Alternatively, or in addition, an anti-inflammatory agent is an antibiotic or an anti-bacterial agent.

Non-limiting examples of conventional antibacterial agents (antibiotics) include, but are not limited to, gentamicin, ampicillin, amikacin (AK), cefazolin, ceftriaxone, clindamycin, cephalothin, ciprofloxacin, chloramphenicol, ceftazidime (CAZ), cefepime (CPE), erythromycin, trimethoprim/sulfamethoxazole (T/S), gatifloxacin, piperacillin/tazobactam (P/T), aztreonam (AZT), imipenem, levofloxacin, penicillin, oxacillin, nitrofurantoin, linezolid, moxifloxacin, meropenem (MER), tobramycin (TO), ciprofloxacin (CP), tetracycline, vancomycin, rifampin synercid, streptomycin Synergy, colistin (CT) and chloramphenicol (C).

Additional non-limiting examples of conventional antimicrobial agent include polyene-based antifungal agents such as amphotericin, amphotericin B, nystatin and pimaricin, amphotericin B liposomal formulations (AmBisome, Abelcet, Amphocil), azole-based antifungal agents such as fluconazole, itraconazole and ketoconazole, allylamine- or morpholine-based antifungal agents such as allylamines (naftifine, terbinafine), and antimetabolite-based antifungal agents such as 5-fluorocytosine, and fungal cell wall inhibitor such as echinocandins like caspofungin, micafungin and anidulafungin.

Any available antibacterial or antimicrobial agent is contemplated.

Alternatively, or in addition, the anti-inflammatory agent is a regeneration-promoting factor.

According to some of any of the embodiments described herein, the anti-inflammatory agent is hydrophobic.

As used herein throughout, the term “hydrophobic” describes a physical property of a material (a compound, or an agent or drug as described herein) or a portion of a material (e.g., a chemical group or moiety in a compound) which accounts for lack of transient formation of bond(s) with water molecules, and thus for water-immiscibility, and is miscible or dissolvable in organic solvents such as hydrocarbons.

A hydrophobic material or portion of a material (e.g., a chemical group or moiety in a compound) is one that is typically non-charged or non charge-polarized and does not tend to form hydrogen bonds.

Hydrophobic materials dissolve more readily in oil than in water or other hydrophilic solvents. Hydrophobic materials can be determined by, for example, as having Log P higher than 1, when Log P is determined in octanol and water phases.

Hydrophobic materials can alternatively, or in addition, be determined as featuring a lipophilicity/hydrophilicity balance (HLB), according to the Davies method, lower than 9, preferably lower than 6.

Those skilled in the art can readily determine if an anti-inflammatory agent is hydrophobic or not, using publicly available product information, databases, or by determining its Log P by methods well known in the art.

According to some of any of the embodiments described herein, the anti-inflammatory agent is a steroidal anti-inflammatory drug.

According to some of any of the embodiments described herein, the anti-inflammatory agent is a hydrophobic steroidal anti-inflammatory drug.

According to some of any of the embodiments described herein, the anti-inflammatory agent is dexamethasone.

### Hydrophilic Moiety:

According to the present embodiments, the block copolymer and the micellar particle comprise a hydrophilic moiety that comprises an amino acid sequence that is capable of interacting with an inflammatory biomarker.

By “capable of interacting with an inflammatory biomarker” it is meant a moiety, e.g., a peptide as described herein, that has an affinity to the biomarker, by being, for example, a ligand, or a substrate of the biomarker.

By “inflammatory biomarker” it is meant a biological substance, e.g., an enzyme or a receptor, that is upregulated as a result of inflammation, namely, is upregulated in an inflamed tissue. Inflammatory biomarkers include biological substances that are upregulated following inflammation or tissue damage, biological substances that are specifically directed to the site of inflammation through biological processes, or biological substances that perform specific biological functions related to inflammation at the site of inflammation.

By “upregulated” it is meant that the biological substance features abnormal, preferably higher expression (overexpression) and/or a higher activity (e.g., overactivity) in an inflamed tissue, compared to a non-inflamed (healthy) tissue, for example, an expression and/or activity higher by at least 10%, or at least 20%, or at least 30%, or at least 40%, or at least 50%, or at least 60%, or at least 70%, or at least 80%, or at least 90%, or at least 100% or 200%, or more, compared to a non-inflamed (e.g., healthy) tissue.

As used herein, “markers associated with inflammation” or “inflammatory biomarkers” include, but are not limited to CRP, cytokines associated with inflammation, such as members of the interleukin family, including IL-1 through IL-17 that are associated with inflammation, TNF-alpha; B61; certain cellular adhesion molecules, such as for example, e-selectin (also known as ELAM), sICAM, integrins, ICAM-1, ICAM-3, BL-CAM, LFA-2, VCAM-1, NCAM and PECAM; neopterin; serum procalcitonin; calprotectin; leukotriene, thromboxane, isoprostane; CXC chemokine ligand 16 (CXCL16), and CC chemokines (e.g., CCL8, CCL13, CCL20).

Cytokines can be pro-inflammatory cytokines (e.g., IL-1α, IL-1β, IL-2, IL-6, IL-8, IL-12, IFN-γ(45)); or anti-inflammatory (inflammation-suppressive) cytokines (e.g., IL-4, IL-5, IL-10, IL-11, IL-13, TGF-β).

According to some of any of the embodiments described herein, the inflammatory biomarker is an enzyme that is upregulated in an inflamed tissue.

According to some of any of the embodiments described herein, the inflammatory biomarker is a protease that is upregulated in an inflamed tissue, which is also referred to herein and in the art as inflammatory protease.

Inflammatory proteases include proteases that are upregulated following inflammation or tissue damage, proteases that are specifically directed to the site of inflammation through biological processes, or proteases that perform specific biological functions related to inflammation at the site of inflammation. Inflammatory proteases may be expressed at and/or targeted to the site of inflammation. In some embodiments, the inflammatory protease is found at the site of inflammation. Non-limiting examples of an inflammatory protease include, but are not limited to, a membrane bound MMP (e.g., MT1-MMP, MT2-MMP, MT3-MMP, MT4-MMP, MT5-MMP, MT6-MMP), serine protease (e.g., plasmin, cathepsin G), lysosomal cysteine protease (cathepsin B), tryptase, chymase, collagenase (e.g., MMP-1, MMP-8, MMP-13), gelatinase (MMP-2, MMP-9), stromelysin (e.g., MMP-3), or membrane type (e.g., MMP-14). In some embodiments, the inflammatory protease is MMP-1, MMP-2, MMP-3, MMP-7, MMP-9, or MMP-14.

The term “MMP” refers to a matrix metalloprotease. The terms “matrix metalloproteinase-2”, “MMP2”, and “MMP-2” are used according to the plain and ordinary meaning in the art and refer to an enzyme by the same name involved in the breakdown of extracellular matrix (e.g. 72 kDa type IV collagenase also known as matrix metalloproteinase-2 (MMP-2) and gelatinase A is an enzyme that in humans is encoded by the MMP2 gene).

The term “MMP-2” may refer to the nucleotide sequence or protein sequence of human MMP-2 (e.g., Entrez 4313, Uniprot P08253, RefSeq NM_001127891, or RefSeq NP_001121363). The term “MMP-2” includes both the wild-type form of the nucleotide sequences or proteins as well as any mutants thereof. In some embodiments, “MMP-2” is wild-type MMP-2 receptor. In some embodiments, “MMP-2” is one or more mutant forms. The term “MMP-2” XYZ refers to a nucleotide sequence or protein of a mutant MMP-2 wherein the Y numbered amino acid of MMP-2 that normally has an X amino acid in the wildtype, instead has a Z amino acid in the mutant. In embodiments, an MMP-2 is the human MMP-2. In embodiments, the MMP-2 has the nucleotide sequence corresponding to reference number GL700274109. In embodiments, the MMP-2 has the nucleotide sequence corresponding to RefSeq NM_001127891.2. In embodiments, the MMP-2 has the protein sequence corresponding to reference number GI: 189217853. In embodiments, the MMP-2 has the protein sequence corresponding to RefSeq NP_001121363.1.

The terms “matrix metalloproteinase-9”, “MMP9”, and “MMP-9” are used according to the plain and ordinary meaning in the art and refer to an enzyme by the same name involved in the breakdown of extracellular matrix (e.g. a 92 kDa type IV collagenase, 92 kDa gelatinase or gelatinase B (GELB). MMP9 may function as a matrixin, a class of enzymes that belong to the zinc-metalloproteinases family involved in the degradation of the extracellular matrix. The term “MMP-9” may refer to the nucleotide sequence or protein sequence of human MMP-9 (e.g., Entrez 4318. Uniprot P14780, RefSeq NM_004994, or RefSeq NP_004985). The term “MMP-9” includes both the wild-type form of the nucleotide sequences or proteins as well as any mutants thereof. In some embodiments, “MMP-9” is wild-type MMP-9 receptor. In some embodiments, “MMP-9” is one or more mutant forms. The term “MMP-9” XYZ refers to a nucleotide sequence or protein of a mutant MMP-9 wherein the Y numbered amino acid of MMP-9 that normally has an X amino acid in the wildtype, instead has a Z amino acid in the mutant. In embodiments, an MMP-9 is the human MMP-9. In embodiments, the MMP-9 has the nucleotide sequence corresponding to reference number GI:74272286. In embodiments, the MMP-9 has the nucleotide sequence corresponding to RefSeq NM 004994.2. In embodiments, the MMP-9 has the protein sequence corresponding to reference number GL74272287. In embodiments, the MMP-2 has the protein sequence corresponding to RefSeq NP 004985.2.

According to some of any of the embodiments described herein the inflammatory protease is MMP-2 and/or MMP-9.

According to some of any of the embodiments described herein, the hydrophilic moiety is a peptide that comprises an amino acid sequence that is recognizable by the inflammatory biomarker.

According to some embodiments, the inflammatory biomarker is an inflammatory enzyme as described herein, and the peptide comprises an amino acid sequence that is cleavable by the inflammatory enzyme. In some embodiments, the amino acid sequence is or forms a part of a recognition motif of the inflammatory biomarker, e.g., the inflammatory enzyme such as an inflammatory protease as described herein.

According to some of any of the embodiments described herein, the inflammatory protease is MMP as described herein and the hydrophilic moiety comprises an amino acid sequence that is recognizable or cleavable by MMP as described herein. An exemplary such a sequence has SEQ ID NO:1.

In embodiments, the inflammatory protease cleavable amino acid sequence is cleaved between the G (i.e. glycine) and L (i.e. leucine) amino acid residues of the inflammatory protease cleavable amino acid sequence having SEQ ID NO:16.

In some embodiments, the MMP cleavable amino acid sequence is an MMP-2 or MMP-9 cleavable amino acid sequence. In some embodiments, the MMP cleavable amino acid sequence is analogous sequence known to be a substrate of a matrix metalloproteinase (MMP). In some embodiments, the MMP cleavable amino acid sequence is an MMP-9 cleavable amino acid sequence. In some embodiments, the MMP cleavable amino acid sequence is an MMP-2 cleavable amino acid sequence. In some embodiments, the MMP cleavable amino acid sequence is an MMP-9 cleavable amino acid sequence. In some embodiments, the MMP cleavable amino acid sequence is cleaved between the G (i.e. glycine) and L (i.e. leucine) amino acid residues of the MMP cleavable amino acid sequence. In some embodiments, the MMP-2 cleavable amino acid sequence is cleaved between the G (i.e. glycine) and L (i.e. leucine) amino acid residues of the MMP-2 cleavable amino acid sequence. In some embodiments, the MMP-9 cleavable amino acid sequence is cleaved between the G (i.e. glycine) and L (i.e. leucine) amino acid residues of the MMP-9 cleavable amino acid sequence.

According to some of any of the embodiments described herein, the peptide comprises from 6 to 50, or from 6 to 30, or from 6 to 20, amino acid residues, including any intermediate values and subranges therebetween.

Representative amino acid sequences that are cleavable by MMP include, without limitation, those having SEQ ID NO: 1, 3, 4, 12, 14, and 16.

Additional amino acid sequences are described, for example, in WO 2016/172386, which is incorporated herein by reference as if fully set forth herein.

According to some of any of the embodiments described herein, the hydrophilic moiety comprises, in addition to the amino acid sequence that is recognizable or cleavable by the inflammatory biomarker (e.g., MMP), one or more amino acid residues that impart to the moiety its hydrophilic nature. Hydrophilic amino acid residues have Log P as described herein lower than 1 and include, for example, amino acid residues that are positively charged or negatively charged at physiological pH.

### Block Copolymers:

Block copolymers can be regarded as copolymers consisting of regularly or statistically alternating two or more different polymeric blocks that differ in composition or structure. Each polymer block in a block copolymer represents polymerized monomers of one type. The blocks can differ from one another by the chemical composition of the monomers composing each block, for example, by the type and or amount of the pendant groups that substitute the backbone units.

The term “block copolymer” is used in accordance with its ordinary meaning and refers to a molecule including repeating subunits, also commonly referred to as monomers (e.g., polymerizable monomers). In some embodiments, the block copolymer includes a peptide sequence (e.g., an inflammatory protease-cleavable amino acid sequence, as described herein, which may be referred to herein as a peptide-polymer amphiphile (PPA), a brush (e.g., polymers adhered to a surface) peptide-polymer amphiphile, or “amphiphilic block copolymer,” resulting from the composition containing both hydrophilic and hydrophobic portions. Such block copolymers may self-assemble into a polymeric micelle (a micellar particle as described herein).

In some embodiments, the block copolymer includes two or more monomers. In embodiments, the block copolymer includes two or more monomers which are independently unique. In some embodiments, the block copolymer includes two or more monomers in a periodic (e.g., repeating pattern) sequence.

According to some of any of the embodiments described herein, there is provided a block copolymer that comprises at least one first block composed of a first plurality of backbone units covalently linked to one another and at least one second block of a second plurality of backbone units covalently linked to one another,
wherein:
the first block comprises at least one anti-inflammatory agent, as described herein in any of the respective embodiments and any combination thereof, covalently attached, directly or via a linking moiety or group, to at least a portion of the first plurality of backbone units composing the first block; and
the second block comprises at least one hydrophilic moiety covalently attached to at least a portion of the second plurality of backbone units composing the second block, the at least one hydrophilic moiety comprises an amino acid sequence that is capable of interacting with an inflammatory biomarker, as described herein in any of the respective embodiments and any combination thereof.

According to some embodiments, the block copolymer can comprise two or more of the first block. The two or more blocks can be sequential (adjacent to one another) or otherwise distributed in the block copolymer between two other blocks. When two or more first blocks are present, the anti-inflammatory agent attached to the backbone units in each block can be the same or different.

According to some embodiments, the first block comprises a first anti-inflammatory drug attached to a first portion of backbone units in the first block and a second anti-inflammatory drug attached to a second portion of backbone units in the first block, wherein the first and second anti-inflammatory agents are different from one another. According to some embodiments, the block copolymer comprises two or more second blocks. The two or more second blocks can be sequential (adjacent to one another) or otherwise distributed in the block copolymer between two other blocks. When two or more first blocks are present, the hydrophilic moiety is the same or different in each of the second blocks.

According to some embodiments, the second block comprises a first hydrophilic moiety attached to a first portion of backbone units in the second block and a second hydrophilic moiety attached to a second portion of backbone units in the second block, wherein the first and second hydrophilic moieties are different from one another.

When the block copolymer comprises two or more of the first block and two or more of the second block, the block copolymer can comprise alternating first and second blocks, in any order.

By “at least a portion” it is meant that at least 10%, or at least 20%, or at least 30%, or at least 40%, or at least 50%, or at least 60%, or at least 70%, or at least 80%, or at least 90%, or all of the backbone units in a respective block are functionalized, namely, have an anti-inflammatory agent or a hydrophilic moiety as described herein, or any other moiety or agent as described herein, attached thereto.

According to some of any of the embodiments described herein, the block copolymer can further comprise at least one additional block, referred to herein as a third block. The at least one third block is composed of a third plurality of backbone units covalently linked to one another, and comprises at least one additional moiety covalently attached, directly or via a linking moiety or group, to at least a portion of the third plurality of backbone units composing the third block. According to some embodiments, the additional moiety can be, for example, a hydrophobic moiety that is different from the at least one anti-inflammatory agent or an additional hydrophilic moiety that is different from the hydrophilic moiety that comprises an amino acid sequence that is capable of interacting with an inflammatory biomarker, or any other moiety that can provide the copolymer with properties that can promote or facilitate micellar particles formation, and/or with therapeutic or pharmacokinetic advantage.

For example, in cases where the anti-inflammatory agent is not hydrophobic, an inclusion of a hydrophobic moiety within a third block can promote particle formation as described herein.

In such cases, it is preferred that the third block will be adjacent to the first block. For example, in cases where the number of the backbone units in a second block and/or the number of second blocks in the block copolymer is relatively low or does not allow particle formation, an inclusion of a hydrophilic moiety in the third block can promote particle formation as described herein.

It is to be noted in this regard that the number of backbone units in the second block is usually limited, since when a too large second block is included, the interaction with the inflammatory biomarker can be adversely affected.

The block copolymer can include two or more third blocks, which can be sequential or distributed within the block copolymer.

According to some of any of the embodiments described herein, the block copolymer further comprises at least one backbone unit that has a labeling agent attached thereto. The labeling agent can be as described herein in any of the respective embodiments and any combination thereof.

The one or more backbone units that have a labeling agent attached thereto can be randomly distributed in the block copolymer or can form a fourth block.

According to some embodiments, the block copolymer comprises a forth block composed of a forth plurality of backbone units covalently linked to one another, and at least one labeling agent covalently attached, directly or via a linking moiety or group, to at least a portion of the forth plurality of backbone units composing the fourth block.

The block copolymer can include two or more third blocks, which can be sequential or distributed within the block copolymer.

According to some of any of the embodiments described herein, the backbone unit(s) that comprise a labeling agent attached thereto, or the one or more fourth block, are adjacent to the second block. In such cases, the labeling agent is included in the shell of the particle formed of the block copolymer.

A block copolymer as described herein is also referred to herein interchangeably as PPA.

It is to be noted that embodiments of the present invention also encompass blend copolymers, which comprise a first and second pluralities of backbone units, as described herein for the first and second blocks, respectively, and optionally a third and fourth plurality of backbone units, as described herein for the third and fourth blocks, and these backbone units are not arranged as blocks but are randomly distributed in the copolymer in any order.

According to some of any of the embodiments described herein, the block copolymers of the present embodiments can be collectively represented by Formula I:

Q1-(L_{q1})-[(A1)(-L₁ₐ-D₁ₐ))ₘ₁-(A2(-L_{1b}-D_{1b}-D_{1b}))ₘ₂]_{z1}-[(B1(-L₂ₐ-P₁ₐ)ₙ₁-(B2(L_{2b}-P_{1b}) ₙ₂]_{z2}-[(C1(-L₃ₐ-M₁ₐ)ₓ₁-(C2(-L_{3b}-M_{1b})ₓ₂]_{z3}-[(E1-(L₄ₐ-Yₐ)_{y1}-(E2-(L_{4b}-Y_{1b})_{y2}]_{z4}-(L_{q2})-Q2 Formula I
wherein:
Q1 and Q2 are each independently a terminal backbone group;
L_{q1} and L_{q2} are each independently a linking moiety or group or absent;
z1 is a positive integer representing the number of the at least one first block(s) in the block copolymer;
[(A1(-L₁ₐ-D₁ₐ))ₘ₁-(A2(-L_{1b}-D_{1b}))ₘ₂] represents the at least one first block wherein:
A1 and A2 are each independently the first plurality of backbone units composing the first block;
L₁ₐ and L_{1b} are each independently a linking group or moiety or absent;
D₁ₐ and D_{1b} are each independently the anti-inflammatory agent;
(A1(-L₁ₐ-D₁ₐ)) represents a first portion of the first plurality of backbone units composing the first block;
m1 is a positive integer representing the number backbone units in the first portion of the first plurality of backbone units composing the first block;
(A2(-L_{1b}-D_{1b})) represents a second portion of the first plurality of backbone units composing the first block;
m2 is 0 or a positive integer representing the number backbone units in the second portion of the first plurality of backbone units composing the first block;
z2 is a positive integer representing the number of the at least one second block(s) in the block copolymer;
[(B1(-L₂ₐ-P₁ₐ)ₙ₁—(B2(L_{2b}-P_{1b})ₙ₂] represents the at least one first block wherein:
B1 and B2 are each independently the second plurality of backbone units composing the second block;
L₂ₐ and L_{2b} are each independently a linking group or moiety or absent;
P₁ₐ and P_{1b} are each independently the hydrophilic moiety that comprises an amino acid sequence that is capable of interacting with an inflammatory biomarker;
(B1(-L₂ₐ-P₁ₐ)) represents a first portion of the second plurality of backbone units composing the second block;
n1 is a positive integer representing the number backbone units in the first portion of the second plurality of backbone units composing the second block;
(B2(-L_{2b}-P_{1b})) represents a second portion of the second plurality of backbone units composing the second block;
n2 is 0 or a positive integer representing the number backbone units in the second portion of the second plurality of backbone units composing the second block;
z3 is 0 or a positive integer representing the number of at least one third block in the block copolymer;
[(C1(-L₃ₐ-M₁ₐ))ₓ₁-(C2(-L_{3b}-M_{1b}))ₓ₂] represents the at least one third block wherein:
C1 and C2 are each independently a third plurality of backbone units composing the third block;
L₃ₐ and L_{3b} are each independently a linking group or moiety or absent;
   M₁ₐ and M_{1b} are each independently an additional moiety as described herein for the third block;
(C1(-L₃ₐ-M₁ₐ)) represents a first portion of the third plurality of backbone units composing the third block;
x1 is a positive integer representing the number backbone units in the first portion of the third plurality of backbone units composing the third block;
(C2(-L_{3b}-M_{1b})) represents a second portion of the third plurality of backbone units composing the third block;
x2 is 0 or a positive integer representing the number backbone units in the second portion of the third plurality of backbone units composing the third block;
z4 is 0 or a positive integer representing the number of at least one fourth block in the block copolymer;
[(E1(-L₄ₐ-Y₁ₐ))_{y1}-(E2(-L_{4b}-Y_{1b}))_{y2}] represents the at least one fourth block wherein:
E1 and E2 are each independently a fourth plurality of backbone units composing the fourth block;
L₄ₐ and L_{4b} are each independently a linking group or moiety or absent;
Y₁ₐ and Y_{1b} are each independently a labeling agent;
(E1(-L₁ₐ-Y₁ₐ)) represents a first portion of the fourth plurality of backbone units composing the fourth block;
y1 is a positive integer representing the number backbone units in the first portion of the fourth plurality of backbone units composing the fourth block;
(E2(-L_{4b}-Y_{1b})) represents a second portion of the fourth plurality of backbone units composing the fourth block; and
y2 is 0 or a positive integer representing the number backbone units in the second portion of the fourth plurality of backbone units composing the fourth block,
wherein the at least one first block, the at least one second block, the at least one third block, if present, and the at least one fourth block, if present, are randomly arranged in the block co-polymer.

According to some of any of the embodiments described herein for Formula I, m2, n2, x2 and y2 are each independently 0, such that respective portion of backbone units is absent, or a positive integer of from 1 to 100, or from 1 to 80, or from 1 to 50, or from 1 to 30, or from 1 to 20, or from 4 to 50, or from 4 to 40, or from 4 to 30, including any intermediate values and subranges therebetween.

According to some of any of the embodiments described herein for Formula I, m1 and n1 are each independently a positive integer of from 1 to 100, or from 1 to 80, or from 1 to 50, or from 1 to 30, or from 1 to 20, or from 4 to 50, or from 4 to 40, or from 4 to 30, including any intermediate values and subranges therebetween.

According to some of any of the embodiments described herein for Formula I, a total number of m1 and m2 ranges from 1 to 100, or from 1 to 80, or from 1 to 50, or from 1 to 30, or from 1 to 20, or from 4 to 50, or from 4 to 40, or from 4 to 30, including any intermediate values and subranges therebetween.

According to some of any of the embodiments described herein for Formula I, a total number of n1 and n2 ranges from 1 to 20, preferably from or from 1 to 10, or from 2 to 10, or from 4 to 10, including any intermediate values and subranges therebetween.

According to some of any of the embodiments described herein for Formula I, a ratio between the total number of m1 and m2 to the total number of n1 and n2 ranges from 100:1 to 1:100, or from 50:1 to 1:50, or from 20:1 to 1:20, or from 10:1 to 1:10, or from 5:1 to 1:5, or from 1:2 to 2:1, or from 100:1 to 1:1, or from 50:1 to 1:1, or from 20:1 to 1:1, or from 10:1 to 1:1, or from 5:1 to 1:1, or from 2:1 to 1:1. The values of n1, n2, m1, m2, z1 and z2 can be manipulated by controlling the synthesis protocol and parameters, as is well accepted for living/controlled polymerization such as ROMP.

According to some of any of the embodiments described herein for Formula I, x1 or a total number of x1 and x2 is a positive integer of from 1 to 100, or from 1 to 80, or from 1 to 50, or from 1 to 30, or from 1 to 20, or from 4 to 50, or from 4 to 40, or from 4 to 30, including any intermediate values and subranges therebetween. The values of x1 or of the total of x1 and x2 can be determined as desired, in accordance with the desirable properties of the block copolymer, as discussed herein.

According to some of any of the embodiments described herein for Formula I, y1 is a positive integer of from 1 to 10, or is 1. When y1 is 1 and y2 is absent, it relates to embodiments where a labeling agent is attached to one or more backbone units. In some embodiments, one of the backbone units having the labeling agent attached thereto is a terminal backbone unit of the block copolymer.

According to some of any of the embodiments described herein for Formula I, z1 and z2 are each independently a positive integer of from 1 to 10, or from 1 to 5, or from 1 to 2, or each is 1, wherein when z1 is an integer of from 2 to 10, each of the first blocks can be the same or different, and when z2 is an integer of from 2 to 10, each of the second blocks can be the same or different.

According to some of any of the embodiments described herein for Formula I, a ratio of z1 to z2 ranges from 100:1 to 1:100, or from 50:1 to 1:50, or from 20:1 to 1:20, or from 10:1 to 1:10, or from 5:1 to 1:5, or from 1:2 to 2:1, or from 100:1 to 1:1, or from 50:1 to 1:1, or from 20:1 to 1:1, or from 10:1 to 1:1, or from 5:1 to 1:1, or from 2:1 to 1:1.

According to some of any of the embodiments described herein for Formula I, z3 is 0 or a positive integer of from 1 to 10, or from 1 to 5, or from 1 to 2, or is 1, wherein when z3 is an integer of from 2 to 10, each of the third blocks can be the same or different, as described herein.

According to some of any of the embodiments described herein for Formula I, z4 is 0 or a positive integer of from 1 to 10, or from 1 to 5, or from 1 to 2, or is 1, wherein when z4 is an integer of from 2 to 10, each of the fourth blocks can be the same or different.

According to some of any of the embodiments described herein for Formula I, when z4 is a positive integer, the block copolymer is arranged such that at least one fourth block is adjacent to the at least one second block.

According to some of any of the embodiments described herein for Formula I, A1 and A2 are different from one another.

According to some of any of the embodiments described herein for Formula I, D₁ₐ and D_{1b} are different from one another.

According to some of any of the embodiments described herein for Formula I, L₁ₐ and L_{2b} are different from one another.

According to some of any of the embodiments described herein for Formula I, B1 and B2 are different from one another.

According to some of any of the embodiments described herein for Formula I, P₁ₐ and P_{1b} are different from one another.

According to some of any of the embodiments described herein for Formula I, L₂, and L₂ₕ are different from one another.

According to some of any of the embodiments described herein for Formula I, C1 and C2 are different from one another.

According to some of any of the embodiments described herein for Formula I, M₁ₐ and M_{1b} are different from one another.

According to some of any of the embodiments described herein for Formula I, L₃ₐ and L_{3b} are different from one another.

According to some of any of the embodiments described herein for Formula I, E1 and E2 are different from one another.

According to some of any of the embodiments described herein for Formula I, Y₁ₐ and Y_{1b} are different from one another.

According to some of any of the embodiments described herein for Formula I, L₄ₐ and L_{4b} are different from one another.

The block copolymer's terminal groups, Q1 and Q2 can each independently comprise independently at least two of hydrogen, alkyl, cycloalkyl, alkenyl, alkoxy, thioalkoxy, aryloxy, amine, a heteroalicyclic, heteroaryl, aryl, a labeling agent and a therapeutically active agent.

In exemplary embodiments, Q1 comprises one or more of hydrogen and aryl, alkyl, or cycloalkyl, for example a phenyl.

In exemplary embodiments, Q2 comprises two hydrogen atoms, such that the copolymer ends by an alkene group.

According to some of any of the embodiments described herein, each of the linking moieties or groups, for example, each L_{q1}, L_{q2}, L₁ₐ, L_{1b}, L₂ₐ, L_{2b}, L₃ₐ, L_{3b}, L₄ₐ and L_{4b} is Formula I, is independently selected from —O—, —S—, —NH—, —C(═O)—, —C(═O)O—, —C(═O)NH—, alkylene, alkoxy, aryloxy, thioalkoxy, cycloalkyl, heteroalicyclic, aryl and heteroaryl.

According to some of any of the embodiments described herein, each backbone unit of the first plurality of backbone units of the at least one first block, second plurality of backbone units of the at least one second block, third plurality of backbone units of the at least one third block, if present, and fourth plurality of backbone units of at least one the fourth block, if present, is independently a ROMP-polymerized monomer, derived from a ROMP-polymerizable monomer.

The term “polymerizable monomer” is used in accordance with its meaning in the art of polymer chemistry and refers to a compound that may covalently bind chemically to other monomer molecules (such as other polymerizable monomers that are the same or different) to form a polymer thereby forming a polymerized monomer. An example of a polymerizable monomer is a ROMP polymerizable monomer, which is a polymerizable monomer capable of binding chemically to other ROMP polymerizable monomers through a ROMP chemical reaction (ring-opening metathesis polymerization) to form a polymer. It will be understood that a polymerizable monomer may be chemically modified in the polymerization reaction to differ from the free polymerizable monomer when forming the polymerized monomer moiety. In embodiments, the ROMP polymerizable monomer includes an olefin. In embodiments, the ROMP polymerizable monomer includes a cyclic olefin. In some embodiments, the ROMP polymerizable monomer includes a cyclic olefin with ring strain (e.g., norbomene or cyclopentene or derivatives thereof). In some of any of the embodiments described herein, the ROMP-polymerizable monomer has a respective moiety (anti-inflammatory agent, a hydrophilic moiety, a labeling agent, or a hydrophilic or hydrophobic moiety as described herein) attached thereto. In some embodiments, the ROMP polymerizable monomer is or includes a substituted or unsubstituted norbornenyl.

Any cyclic olefin (unsaturated cyclic compounds) suitable for the metathesis reactions disclosed herein may be used.

Herein, the phrases “cyclic olefin” and “unsaturated cyclic compound” are used interchangeably encompasses compounds comprising one, two, three or more non-aromatic rings (fused and/or unfused rings) which comprise at least one pair of adjacent carbon atoms in the ring which are bound to one another by an unsaturated bond. The ring may optionally be substituted or unsubstituted, and the cyclic olefin may optionally comprise one unsaturated bond (“monounsaturated”), two unsaturated bonds (“di-unsaturated”), three unsaturated bond (“tri-unsaturated”), or more than three unsaturated bonds. When substituted, any number of substituents may be present (optionally from 1 to 5, and optionally 2, 3, 4 or 5 substituents), and the substituent(s) may optionally be any substituent describes herein as being optionally attached to an alkyl or alkenyl.

Examples of cyclic olefins include, without limitation, cyclooctene, cyclododecene, and (c,t,t)-1,5,9-cyclododecatriene.

Examples of cyclic olefins with more than one ring include, without limitation, norbomene, dicyclopentadiene, tricyclopentadiene, and 5-ethylidene-2-norbornene.

The cyclic olefin may be a strained or unstrained cyclic olefin, provided the cyclic olefin is able to participate in a ROMP reaction either individually or as part of a ROMP cyclic olefin composition. While certain unstrained cyclic olefins such as cyclohexene are generally understood to not undergo ROMP reactions by themselves, under appropriate circumstances, such unstrained cyclic olefins may nonetheless be ROMP active. For example, when present as a co-monomer in a ROMP composition, unstrained cyclic olefins may be ROMP active. Accordingly, as used herein and as would be appreciated by the skilled artisan, the term “unstrained cyclic olefin” is intended to refer to those unstrained cyclic olefins that may undergo a ROMP reaction under any conditions, or in any ROMP composition, provided the unstrained cyclic olefin is ROMP active.

In some embodiments of any one of the embodiments described herein, the substituted or unsubstituted cyclic olefin comprises from 5 to 24 carbon atoms. In some such embodiments, the cyclic olefin is a hydrocarbon devoid of heteroatoms. In alternative embodiments, the cyclic olefin comprises one or more (e.g., from 2 to 12) heteroatoms such as O, N, S, or P, for example, crown ether cyclic olefins which include numerous O heteroatoms throughout the cycle, are within the scope of the invention.

In some embodiments of any one of the embodiments described herein relating to a cyclic olefin comprising from 5 to 24 carbon atoms, the cyclic olefin is mono-unsaturated, di-unsaturated, or tri-unsaturated.

In some embodiments of any of the embodiments described herein, the cyclic olefin is monocyclic.

In some embodiments of any of the embodiments described herein, the cyclic olefin is monounsaturated, optionally being both monocyclic and monounsaturated.

Examples of monounsaturated, monocyclic olefins include, without limitation, cyclopentene, cyclohexene, cycloheptene, cyclooctene, cyclononene, cyclodecene, cycloundecene, cyclododecene, tricyclodecene, tetracyclodecene, octacyclodecene, and cycloeicosene, and substituted versions thereof such as methylcyclopentene (e.g., 1-methylcyclopentene, 4-methylcyclopentene), ethylcyclopentene (e.g., 1-ethylcyclopentene), isopropylcyclohexene (e.g., 1-isopropylcyclohexene), chloropentene (e.g., 1-chloropentene), fluorocyclopentene (e.g., 1-fluorocyclopentene), methoxycyclopentene (e.g., 4-methoxy-cyclopentene), ethoxycyclopentene (e.g., 4-ethoxy-cyclopentene), cyclopentene-thiol (e.g., cyclopent-3-ene-thiol), methylsulfanyl-cyclopentene (e.g., 4-methylsulfanyl-cyclopentene), methylcyclohexene (e.g., 3-methylcyclohexene), methylcyclooctene (e.g., 1-methylcyclooctene), and dimethylcyclooctene (e.g., 1,5-dimethylcyclooctene).

In some embodiments of any of the embodiments described herein, the cyclic olefin is diunsaturated, optionally being both monocyclic and diunsaturated.

Examples of diunsaturated, monocyclic olefins include, without limitation, 1,3-cyclopentadiene, 1,3-cyclohexadiene, 1,4-cyclohexadiene, heptadiene (e.g., 1,3-cycloheptadiene), octadiene (e.g., 1,5-cyclooctadiene, 1,3-cyclooctadiene), and substituted versions thereof (e.g., 5-ethyl-1,3-cyclohexadiene).

In some embodiments of any of the embodiments described herein, the cyclic olefin comprises more than two (optionally three) unsaturated bonds.

In some embodiments of any of the embodiments described herein, the cyclic olefin is polycyclic.

Herein, the term “polycyclic” refers to a structure comprising two or more fused rings.

An exemplary polycyclic olefin is norbornene.

As used herein, the term “norbornene” refers to any compound that includes at least one substituted or unsubstituted bicyclo[2.2.1]hept-2-ene moiety or dehydrogenated derivative thereof, including without limitation, bicyclo[2.2.1]hept-2-ene (referred to in the art as “norbornene”) and substituted versions thereof, norbornadiene, (bicyclo[2.2.1]hepta-2,5-diene) and substituted versions thereof, and polycyclic norbornenes, and substituted versions thereof.

Examples of bicyclic and polycyclic olefins include, without limitation, dicyclopentadiene (DCPD); trimer and higher order oligomers of cyclopentadiene (e.g., cyclopentadiene tetramer, cyclopentadiene pentamer); ethylidenenorbomene; dicyclohexadiene; norbornene; 5-methyl-2-norbornene; 5-ethyl-2-norbornene; 5-isobutyl-2-norbornene; 5,6-dimethyl-2-norbornene; 5-phenylnorbomene; 5-benzylnorbornene; 5-acetylnorbornene; 5-methoxycarbonylnorbornene; 5-ethyoxycarbonyl-l-norbornene; 5-methyl-5-methoxy-carbonylnorbornene; 5-cyanonorbornene; 5,5,6-trimethyl-2-norbornene; cyclo-hexenylnorbomene; endo,exo-5,6-dimethoxynorbornene; endo,endo-5,6-dimethoxynorbomene; endo,exo-5,6-dimethoxycarbonylnorbornene; endo,endo-5,6-dimethoxycarbonylnorbomene; 2,3-dimethoxynorbornene; norbornadiene; tricycloundecene; tetracyclododecene; 8-methyltetracyclododecene; 8-ethyltetracyclododecene; 8-methoxycarbonyltetracyclododecene; 8-methyl-8-tetracyclododecene; 8-cyanotetracyclododecene; pentacyclopentadecene; pentacyclohexadecene; and the like, and their structural isomers, stereoisomers, and mixtures thereof.

Additional examples of bicyclic and polycyclic olefins include, without limitation, C₂-C₁₂-alkyl-substituted and C₂-C₁₂-alkenyl-substituted norbomenes, for example, 5-butyl-2-norbornene, 5-hexyl-2-norbornene, 5-octyl-2-norbornene, 5-decyl-2-norbornene, 5-dodecyl-2-norbornene, 5-vinyl-2-norbornene, 5-ethylidene-2-norbornene, 5-isopropenyl-2-norbornene, 5-propenyl-2-norbornene, and 5-butenyl-2-norbornene, and the like.

In some embodiments of any of the embodiments described herein, the cyclic olefin is dicyclopentadiene; tricyclopentadiene; dicyclohexadiene; norbornene; 5-methyl-2-norbornene; 5-ethyl-2-norbornene; 5-isobutyl-2-norbornene; 5,6-dimethyl-2-norbornene; 5-phenylnorbornene; 5-benzylnorbornene; 5-acetylnorbornene; 5-methoxycarbonylnorbornene; 5-ethoxycarbonyl-1-norbornene; 5-methyl-5-methoxy-carbonylnorbornene; 5-cyanonorbomene; 5,5,6-trimethyl-2-norbornene; cyclo-hexenylnorbomene; endo,exo-5,6-dimethoxynorbomene; endo,endo-5,6-dimethoxynorbornene; endo,exo-5-6-dimethoxycarbonylnorbornene; endo,endo-5,6-dimethoxycarbonylnorbornene; 2,3-dimethoxynorbomene; norbornadiene; tricycloundecene; tetracyclododecene; 8-methyltetracyclododecene; 8-ethyl-tetracyclododecene; 8-methoxycarbonyltetracyclododecene; 8-methyl-8-tetracyclo-dodecene; 8-cyanotetracyclododecene; pentacyclopentadecene; pentacyclohexadecene; an oligomer of cyclopentadiene (e.g., cyclopentadiene tetramer, cyclopentadiene pentamer); and/or a C₂-C₁₂-alkyl-substituted norbornene or C₂-C₁₂-alkenyl-substituted norbornene (e.g., 5-butyl-2-norbornene; 5-hexyl-2-norbornene; 5-octyl-2-norbornene; 5-decyl-2-norbornene; 5-dodecyl-2-norbornene; 5-vinyl-2-norbornene; 5-ethylidene-2-norbornene; 5-isopropenyl-2-norbornene; 5-propenyl-2-norbornene; 5-butenyl-2-norbomene).

In some embodiments of any of the embodiments described herein, the cyclic olefin is dicyclopentadiene, tricyclopentadiene, or higher order oligomer of cyclopentadiene (e.g., cyclopentadiene tetramer, cyclopentadiene pentamer), tetracyclododecene, norbornene, and/or a C₂-C₁₂-alkyl-substituted norbornene or C₂-C₁₂-alkenyl-substituted norbornene (e.g., according to any of the respective embodiments described herein).

According to some of any of the embodiments described herein, the ROMP-polymerized monomer is derived from a substituted or unsubstituted norbornene.

Exemplary norbornenes are described in the Examples section that follows.

The above polymerizable monomers form the polymerized monomers within the block copolymers disclosed herein.

The term “ring-opening metathesis polymerization” or “ROMP” is used in accordance with its meaning in polymer chemistry and refers to a chain-growth polymerization (e.g., olefin metathesis chain-growth polymerization). In some embodiments, the reaction is driven by relief of ring strain in cyclic olefins (e.g., norbornene or cyclopentene). In some embodiments, the ROMP uses a ruthenium catalyst. In some embodiments, the ROMP uses a Grubbs' catalyst (e.g., of a first, second or preferably third, generation). Other ROMP catalysts are contemplated.

According to some of any of the embodiments described herein, the backbone units forming the block copolymer of Formula I can form a non-block copolymer, in each the units are randomly distributed within the backbone chain (form a blend copolymer).

Block (or blend) copolymers as described herein, according to some embodiments, are designed as capable of forming a micellar particle as described herein in any of the respective embodiments, upon contacting an aqueous solution, as described herein. According to some of any of the embodiments described herein, the chemical composition of the copolymers can be readily manipulated by selecting the monomers and the mol ratio therebetween, so as to facilitate particles formulation.

The copolymers as described herein can be prepared by known polymerization methodologies, for example, ROMP. Exemplary synthetic protocols are described in the Examples section that follows.

### Labeling Agents:

As used herein, the phrase “labeling agent” describes a detectable moiety or a probe. Exemplary labeling agents which are suitable for use in the context of the these embodiments include, but are not limited to, a fluorescent agent, a radioactive agent, a magnetic agent, a chromophore, a bioluminescent agent, a chemiluminescent agent, a phosphorescent agent and a heavy metal cluster.

The phrase “radioactive agent” describes a substance (i.e. radionuclide or radioisotope) which loses energy (decays) by emitting ionizing particles and radiation. When the substance decays, its presence can be determined by detecting the radiation emitted by it. For these purposes, a particularly useful type of radioactive decay is positron emission. Exemplary radioactive agents include ^{99m}Tc, ¹⁸F, ¹³¹I and ¹²⁵I.

The term “magnetic agent” describes a substance which is attracted to an externally applied magnetic field. These substances are commonly used as contrast media in order to improve the visibility of internal body structures in Magnetic Resonance Imaging (MRI). The most commonly used compounds for contrast enhancement are gadolinium-based. MRI contrast agents alter the relaxation times of tissues and body cavities where they are present, which, depending on the image weighting, can give a higher or lower signal.

As used herein, the term “chromophore” describes a chemical moiety that, when attached to another molecule, renders the latter colored and thus visible when various spectrophotometric measurements are applied.

The term “bioluminescent agent” describes a substance which emits light by a biochemical process

The term “chemiluminescent agent” describes a substance which emits light as the result of a chemical reaction.

The phrase “fluorescent agent” refers to a compound that emits light at a specific wavelength during exposure to radiation from an external source. Exemplary such labeling agents include agents that emit light at the Near IR range (e.g., cyanines).

The phrase “phosphorescent agent” refers to a compound emitting light without appreciable heat or external excitation as by slow oxidation of phosphorous.

A heavy metal cluster can be for example a cluster of gold atoms used, for example, for labeling in electron microscopy techniques.

According to some of any of the embodiments described herein, the labeling agent is a fluorescent agent, and in some embodiments it is a NIR-fluorescent agent such as fluorescein labels, Alexa Fluor dyes, and cyanine dyes such as Cy2, Cy3, Cy5, Cy5.5, Cy7, optionally substituted coumarin, R-phycoerythrin, allophycoerythrin, Texas Red and Princeston Red. Any other agents are contemplated.

### Micellar Particle:

According to some of any of the embodiments described herein, there is provided a micellar particle that comprises a plurality of block (or blend) copolymers as described herein in any of the respective embodiments arranged such that the particle comprises a core comprising the first block (namely, the anti-inflammatory agent, and optionally a hydrophobic moiety of a third block) and a hydrophilic shell comprising the second block (namely the hydrophilic moiety as described herein, and optionally an additional hydrophilic moiety of a third block). According to some embodiments, the micellar particle further comprises a labeling agent as described herein, which can be in the core and/or in the shell, and is preferably in the shell.

The phrase “micellar particle”, which is also referred to herein interchangeably as “polymeric micelle” refers to a micelle including the block copolymers as described herein. The internal portion (e.g. core) of the polymeric micelle (also referred to herein as a “block copolymer micelle”) is hydrophobic while the exterior portion (e.g. shell) is hydrophilic. In embodiments, the micellar particle is a nanoparticle (referred to herein as “micellar nanoparticle” or “polymeric micelle nanoparticle” or “NP” or “PPA NP”). In some embodiments, the polymeric micelle is a spherical nanoparticle (referred to herein as a “spherical polymeric micelle nanoparticle” or “spherical micellar nanoparticle”).

A “nanoparticle,” as used herein, is a particle which has at least one dimension (e.g., a diameter) that is less than or equal to 1000 nanometers, e.g., of from 0.1 to 1,000, or from 0.1 to 500, or from 0.1 to 200, or from 0.1 to 100, or from 1 to 1,000, or from 1 to 500, or from 1 to 200, or from 1 to 100, or from 10 to 100, or from 10 to 50, or from 1 to 50, nanometers, including any intermediate values and subranges therebetween. In some embodiments, the nanoparticle constructs provided herein may have a substantially spherical shape (referred to herein as a “spherical nanoparticle”).

According to some of any of the embodiments described herein, there is provided a method of forming the micellar particle as described herein, which comprises contacting the plurality of block copolymers with an aqueous solution.

Exemplary procedures are described in the Examples section that follows.

In an exemplary procedure, a copolymer as described herein as described herein is dissolved in a water-miscible or partially water-miscible organic solvent (e.g., DMSO), at a concentration that ranges from about 1 to about 10 mg/mL. An aqueous solution, such a buffer, is then added gradually and the solution is mixed at a temperature that ranges from 0 to 37° C. until particles are formed. The particles solution can be filtered and/or concentrated.

According to some of any of the embodiments described herein, the micellar particle, or a plurality thereof, is capable of forming a polymeric aggregate upon contacting an inflammatory biomarker as described herein. Such a particle is also referred to herein as “responsive”.

A “responsive particle” and the like refer to a particle which contains a plurality of the block copolymers as described herein. A micellar particle as described herein is capable of undergoing a morphological transition from a discrete material (e.g., spherical-shaped micellar nanoparticle). In embodiments, the morphological transition is from a discrete material to a network-like assembly in response to environmental stimuli (e.g., enzymatic peptide cleavage).

The terms “polymeric aggregate,” “micron scale aggregate”, “amphiphilic aggregate” and “aggregate assembly” are used herein synonymously in the context of a plurality of nanoparticles or copolymers disclosed herein which coalesce into network-like assemblies in response to an environmental stimuli (e.g., enzymatic peptide cleavage). For example, a polymeric micelle forms a disordered structure following cleavage of the MMP cleavable moiety. The disordered structure may then aggregate to form a network-like assembly (e.g. aggregation including hydrophobic interactions).

According to an aspect of some embodiments of the present invention, there is provided a polymeric aggregate as described herein, formed of a plurality of micellar particles as described herein in any of the respective embodiments, upon contacting an inflammatory marker, as described herein.

### Pharmaceutical Compositions:

The micellar particle of some embodiments of the invention can be administered to an organism or otherwise utilized per se, or as forming a part of a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

According to an aspect of some embodiments of the present invention there is provided a pharmaceutical composition comprising a micellar particle as described herein in any of the respective embodiments, or a plurality of such particles, and a pharmaceutically acceptable carrier.

According to some of any of the embodiments described herein, the pharmaceutical composition is for use in treating chronic inflammation in a subject in need thereof, as described herein.

According to some of any of the embodiments described herein, the pharmaceutical composition is formulated for local administration at or a in close vicinity to, an inflamed tissue in the subject, as described herein.

According to some of any of the embodiments described herein, the pharmaceutical composition is formulated for local administration by injection.

According to some of any of the embodiments described herein, the carrier is an aqueous carrier.

Herein the term “active ingredient” refers to the micellar particles as described herein in any of the respective embodiments.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences”, Mack Publishing Co., Easton, PA, latest edition, which is incorporated herein by reference.

Suitable routes of administration may, for example, include oral, rectal, topical, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

Exemplary routes or modes of administration include, without limitation, oral, rectal, transmucosal, intestinal, parenteral, intrathecal, direct intraventricular, intravenous, intraperitoneal, intramuscular, intranasal, and intraocular routes.

For topical administration, an appropriate carrier may be selected and optionally other ingredients that can be included in the composition, as is detailed herein. Hence, the compositions can be, for example, in a form of a cream, an ointment, a paste, a gel, a lotion, and/or a soap.

Ointments are semisolid preparations, typically based on vegetable oil (e.g., shea butter and/or cocoa butter), petrolatum or petroleum derivatives. As with other carriers or vehicles, an ointment base should be inert, stable, nonirritating and non-sensitizing.

Lotions are preparations that may to be applied to the skin without friction. Lotions are typically liquid or semiliquid preparations with a water or alcohol base, for example, an emulsion of the oil-in-water type. Lotions are typically preferred for treating large areas (e.g., as is frequently desirable for sunscreen compositions), due to the ease of applying a more fluid composition.

Creams are viscous liquids or semisolid emulsions, either oil-in-water or water-in-oil. Cream bases typically contain an oil phase, an emulsifier and an aqueous phase. The oil phase, also called the “lipophilic” phase, optionally comprises petrolatum and/or a fatty alcohol such as cetyl or stearyl alcohol. The aqueous phase optionally contains a humectant. The emulsifier in a cream formulation is optionally a nonionic, anionic, cationic or amphoteric surfactant.

Herein, the term “emulsion” refers to a composition comprising liquids in two or more distinct phases (e.g., a hydrophilic phase and a lipophilic phase). Non-liquid substances (e.g., dispersed solids and/or gas bubbles) may optionally also be present.

As used herein and in the art, a “water-in-oil emulsion” is an emulsion characterized by an aqueous phase which is dispersed within a lipophilic phase.

As used herein and in the art, an “oil-in-water emulsion” is an emulsion characterized by a lipophilic phase which is dispersed within an aqueous phase.

Pastes are semisolid dosage forms which, depending on the nature of the base, may be a fatty paste or a paste made from a single-phase aqueous gel. The base in a fatty paste is generally petrolatum, hydrophilic petrolatum, and the like. The pastes made from single-phase aqueous gels generally incorporate carboxymethylcellulose or the like as a base.

Gel formulations are semisolid, suspension-type systems. Single-phase gels optionally contain organic macromolecules distributed substantially uniformly throughout the carrier liquid, which is typically aqueous; but also, preferably, contains a non-aqueous solvent, and optionally an oil. Preferred organic macromolecules (e.g., gelling agents) include crosslinked acrylic acid polymers such as the family of carbomer polymers, e.g., carboxypolyalkylenes, that may be obtained commercially under the trademark Carbopol®. Other types of preferred polymers in this context are hydrophilic polymers such as polyethylene oxides, polyoxyethylene-polyoxypropylene copolymers and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

A composition formulated for topical administration may optionally be present in a patch, a swab, a pledget, and/or a pad.

Dermal patches and the like may comprise some or all of the following components: a composition to be applied (e.g., as described herein); a liner for protecting the patch during storage, which is optionally removed prior to use; an adhesive for adhering different components together and/or adhering the patch to the skin; a backing which protects the patch from the outer environment; and/or a membrane which controls release of a drug to the skin.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (micellar particle) effective to prevent, alleviate or ameliorate symptoms of a disorder (e.g., inflammation) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See, for example, Fingl, et al., 1975, in “The Pharmacological Basis of Therapeutics”, Ch. 1, p. 1).

Dosage amount and interval may be adjusted individually to provide levels of the active ingredient that are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

According to some of any of the embodiments described herein, a micellar particle as described herein or a composition comprising same, as described herein, are administered to a subject afflicted by a chronic inflammation, and hence repeated administrations are desirable. According to some embodiments, a time interval between administrations is at least one day, preferably at least 3, 4, 5 or more days, or at least one week, or at least 2, 3, 4, or more weeks, or at least one month, whereby longer intervals are also contemplated.

According to some of any of the embodiments described herein, treatment using a micellar particle as described herein or a composition comprising same, as described herein, is effected by administering the particle or composition once a day, or once every 2, 3, 4 or more days, or once a week, or once every 2, 3, or more weeks, or once a month, or once every 2, 3 or more months.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein, the term “subject” includes mammals, preferably human beings at any age which suffer from the pathology. Preferably, this term encompasses individuals who are at risk to develop the pathology.

For any of the embodiments described herein, the compound described herein (a micellar particle as described herein and a copolymer as described herein or any of the moieties or agents as described herein) may be in a form of a salt, for example, a pharmaceutically acceptable salt, and/or in a form of a prodrug.

As used herein, the phrase “pharmaceutically acceptable salt” refers to a charged species of the parent compound (a micellar particle as described herein and a copolymer as described herein or any of the moieties or agents as described herein) and its counter-ion, which is typically used to modify the solubility characteristics of the parent compound (a micellar particle as described herein and a copolymer as described herein or any of the moieties or agents as described herein) and/or to reduce any significant irritation to an organism by the parent compound (a micellar particle as described herein and a copolymer as described herein or any of the moieties or agents as described herein), while not abrogating the biological activity and properties of the compound (a micellar particle as described herein and a copolymer as described herein or any of the moieties or agents as described herein). A pharmaceutically acceptable salt of a compound as described herein (a micellar particle as described herein and a copolymer as described herein or any of the moieties or agents as described herein) can alternatively be formed during the synthesis of the compound, e.g., in the course of isolating the compound from a reaction mixture or re-crystallizing the compound.

In the context of some of the present embodiments, a pharmaceutically acceptable salt of the compounds described herein (a micellar particle as described herein and a copolymer as described herein or any of the moieties or agents as described herein) may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound (a micellar particle as described herein and a copolymer as described herein or any of the moieties or agents as described herein) which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein (a micellar particle as described herein and a copolymer as described herein or any of the moieties or agents as described herein) can be used as polymorphs and the present embodiments further encompass any isomorph of the compounds and any combination thereof.

The compounds and structures described herein (a micellar particle as described herein and a copolymer as described herein or any of the moieties or agents as described herein) encompass any stereoisomer, including enantiomers and diastereomers, of the compounds described herein, unless a particular stereoisomer is specifically indicated.

As used herein, the term “enantiomer” refers to a stereoisomer of a compound (a micellar particle as described herein and a copolymer as described herein or any of the moieties or agents as described herein) that is superposable with respect to its counterpart only by a complete inversion/reflection (mirror image) of each other. Enantiomers are the to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

Herein, the term “peptide” refers to a polymer comprising at least 4 amino acid residues linked by peptide bonds or analogs thereof (as described herein below), and optionally only by peptide bonds per se. In some embodiments, the peptide comprises at least 10 amino acid residues or analogs thereof. In some embodiments, the polypeptide comprises up to 100, preferably up to 50, more preferably up to 20 amino acid residues or analogs thereof.

The term “peptide” encompasses native peptides (e.g., degradation products, synthetically synthesized peptides and/or recombinant peptides), including, without limitation, native proteins, fragments of native proteins and homologs of native proteins and/or fragments thereof; as well as peptidomimetics (typically, synthetically synthesized peptides) and peptoids and semipeptoids which are peptide analogs, which may have, for example, modifications rendering the peptides more stable while in a body or more capable of penetrating into cells. Such modifications include, but are not limited to N-terminus modification, C-terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided herein below.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (-NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g., fatty acids, complex carbohydrates etc).

The term “amino acid” or “amino acids” is understood to include the 20 naturally occurring amino acids; those amino acids often modified post-translationally in-vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables A and B below list naturally occurring amino acids (Table A), and non-conventional or modified amino acids (e.g., synthetic, Table B) which can be used with some embodiments of the invention.

TABLE A

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE B

| Non-conventional amino acid | Code | Non-conventional amino acid | Code |
|---|---|---|---|
| ornithine | Orn | hydroxyproline | Hyp |
| α-aminobutyric acid | Abu | aminonorbornyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentyl-alanine | Cpen | N-methyl-cyclopentylalanine | Nmcpen |
| α-aminoα-methylbutyrate | Aabu | N-methyl-α-amino-α-methylbutyrate | Nmaabu |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) carbamylmethyl-glycine | Nnbhm |
| N-(3,3-diphenylpropyl)glycine | Nbhe | N-(N-(3,3-diphenylpropyl) carbamylmethyl-glycine | Nnbhe |
| 1-carboxy-1-(2,2-diphenyl ethylamino)cyclopropane | Nmbc | 1,2,3,4-tetrahydroisoquinoline-3-carboxylic acid | Tic |
| phosphoserine | pSer | phosphothreonine | pThr |
| phosphotyrosine | pTyr | O-methyl-tyrosine | |
| 2-aminoadipic acid | | hydroxylysine | |

The peptides of some embodiments of the invention are preferably utilized in a linear form, although it will be appreciated that in cases where cyclicization does not severely interfere with peptide characteristics, cyclic forms of the peptide can also be utilized.

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptides of some embodiments of the invention involves solid phase peptide synthesis.

Large scale peptide synthesis is described by Andersson (2000) Biopolymers, 55(3), 227-250.

Herein, a “homolog” of a given peptide refers to a peptide that exhibits at least 80% homology, preferably at least 90% homology, and more preferably at least 95% homology, and more preferably at least 98% homology to the given peptide (optionally exhibiting at least 80%, at least 90% identity, at least 95%, or at least 98% sequence identity to the given peptide). In some embodiments, a homolog of a given peptide further shares a therapeutic activity with the given peptide. The percentage of homology refers to the percentage of amino acid residues in a first peptide sequence which matches a corresponding residue of a second peptide sequence to which the first peptide is being compared. Generally, the peptides are aligned to give maximum homology. A variety of strategies are known in the art for performing comparisons of amino acid or nucleotide sequences in order to assess degrees of identity, including, for example, manual alignment, computer assisted sequence alignment and combinations thereof. A number of algorithms (which are generally computer implemented) for performing sequence alignment are widely available, or can be produced by one of skill in the art. Representative algorithms include, e.g., the local homology algorithm of Smith and Waterman (Adv. Appl. Math., 1981, 2: 482); the homology alignment algorithm of Needleman and Wunsch (J. Mol. Biol., 1970, 48: 443); the search for similarity method of Pearson and Lipman (Proc. Natl. Acad. Sci. (USA), 1988, 85: 2444); and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Readily available computer programs incorporating such algorithms include, for example, BLASTN, BLASTP, Gapped BLAST, PILEUP, CLUSTALW etc. When utilizing BLAST and Gapped BLAST programs, default parameters of the respective programs may be used. Alternatively, the practitioner may use non-default parameters depending on his or her experimental and/or other requirements (see for example, the Web site having URL www(dot)ncbi(dot)nlm(dot)nih(dot)gov).

Herein throughout, the phrase “linking moiety” or “linking group” describes a group that connects two or more moieties or groups in a compound. A linking moiety is typically derived from a bi- or tri-functional compound, and can be regarded as a bi- or tri-radical moiety, which is connected to two or three other moieties, via two or three atoms thereof, respectively.

Exemplary linking moieties include a hydrocarbon moiety or chain, optionally interrupted by one or more heteroatoms, as defined herein, and/or any of the chemical groups listed below, when defined as linking groups.

When a chemical group is referred to herein as “end group” it is to be interpreted as a substituent, which is connected to another group via one atom thereof.

Herein throughout, the term “hydrocarbon” collectively describes a chemical group composed mainly of carbon and hydrogen atoms. A hydrocarbon can be comprised of alkyl, alkene, alkyne, aryl, and/or cycloalkyl, each can be substituted or unsubstituted, and can be interrupted by one or more heteroatoms. The number of carbon atoms can range from 2 to 20, and is preferably lower, e.g., from 1 to 10, or from 1 to 6, or from 1 to 4. A hydrocarbon can be a linking group or an end group.

As used herein, the term “amine” describes both a —NR′R″ group and a —NR′— group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

The term “amine” is used herein to describe a —NR′R″ group in cases where the amine is an end group, as defined hereinunder, and is used herein to describe a —NR′— group in cases where the amine is a linking group or is or part of a linking moiety.

The term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms (C(1-4) alkyl). The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking group, as this phrase is defined hereinabove, which connects two or more moieties via at least two carbons in its chain. When the alkyl is a linking group, it is also referred to herein as “alkylene” or “alkylene chain”.

Alkene and Alkyne, as used herein, are an alkyl, as defined herein, which contains one or more double bond or triple bond, respectively.

The term “cycloalkyl” describes an all-carbon monocyclic ring or fused rings (i.e., rings which share an adjacent pair of carbon atoms) group where one or more of the rings does not have a completely conjugated pi-electron system. Examples include, without limitation, cyclohexane, adamantine, norbornyl, isobornyl, and the like. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyrane, morpholino, oxalidine, and the like. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking group, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

The term “halide” and “halo” describes fluorine, chlorine, bromine or iodine.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking group, as these phrases are defined hereinabove, where R′ is as defined hereinabove.

The term “thiosulfate” describes a —O—S(═S)(═O)-OR′ end group or a —O—S(═S)(═O)-O-linking group, as these phrases are defined hereinabove, where R′ is as defined hereinabove.

The term “sulfite” describes an —O—S(═O)—O—R′ end group or a —O—S(═O)—O— group linking group, as these phrases are defined hereinabove, where R′ is as defined hereinabove.

The term “thiosulfite” describes a —O—S(═S)—O—R′ end group or an —O—S(═S)—O— group linking group, as these phrases are defined hereinabove, where R′ is as defined hereinabove.

The term “sulfinate” describes a —S(═O)—OR′ end group or an —S(═O)—O— group linking group, as these phrases are defined hereinabove, where R′ is as defined hereinabove.

The term “sulfoxide” or “sulfinyl” describes a —S(═O)R′ end group or an —S(═O)— linking group, as these phrases are defined hereinabove, where R′ is as defined hereinabove.

The term “sulfonate” describes a —S(═O)₂—R′ end group or an —S(═O)₂— linking group, as these phrases are defined hereinabove, where R′ is as defined herein.

The term “S-sulfonamide” describes a —S(═O)₂—NR′R″ end group or a —S(═O)₂—NR′-linking group, as these phrases are defined hereinabove, with R′ and R″ as defined herein.

The term “N-sulfonamide” describes an R′S(═O)₂—NR″— end group or a —S(═O)₂—NR′-linking group, as these phrases are defined hereinabove, where R′ and R″ are as defined herein.

The term “disulfide” refers to a —S—SR′ end group or a —S—S— linking group, as these phrases are defined hereinabove, where R′ is as defined herein.

The term “phosphonate” describes a —P(═O)(OR′)(OR″) end group or a —P(═O)(OR′)(O)— linking group, as these phrases are defined hereinabove, with R′ and R″ as defined herein.

The term “thiophosphonate” describes a —P(═S)(OR′)(OR″) end group or a —P(═S)(OR′)(O)— linking group, as these phrases are defined hereinabove, with R′ and R″ as defined herein.

The term “phosphinyl” describes a —PR′R″ end group or a —PR′— linking group, as these phrases are defined hereinabove, with R′ and R″ as defined hereinabove.

The term “phosphine oxide” describes a —P(═O)(R′)(R″) end group or a —P(═O)(R′)— linking group, as these phrases are defined hereinabove, with R′ and R″ as defined herein.

The term “phosphine sulfide” describes a —P(═S)(R′)(R″) end group or a —P(═S)(R′)— linking group, as these phrases are defined hereinabove, with R′ and R″ as defined herein.

The term “phosphite” describes an —O—PR′(═O)(OR″) end group or an —O—PH(═O)(O)— linking group, as these phrases are defined hereinabove, with R′ and R″ as defined herein.

The term “carbonyl” or “carbonate” as used herein, describes a —C(═O)—R′ end group or a —C(═O)— linking group, as these phrases are defined hereinabove, with R′ as defined herein.

The term “thiocarbonyl” as used herein, describes a —C(═S)—R′ end group or a —C(═S)— linking group, as these phrases are defined hereinabove, with R′ as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “oxime” describes a ═N—OH end group or a ═N—O— linking group, as these phrases are defined hereinabove.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The “hydroxyalkyl” is also referred to herein as “alcohol”, and describes an alkyl, as defined herein, substituted by a hydroxy group.

The term “cyano” describes a —C≡N group.

The term “cyanurate” describes a

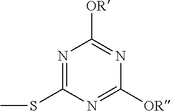

end group or

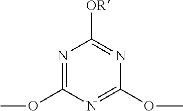

linking group, with R′ and R″ as defined herein.

The term “isocyanurate” describes a

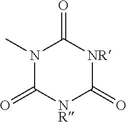

end group or a

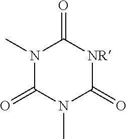

linking group, with R′ and R″ as defined herein.

The term “thiocyanurate” describes a

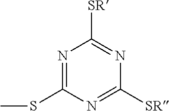

end group or

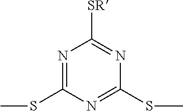

linking group, with R′ and R″ as defined herein.

The term “isocyanate” describes an-N═C═O group.

The term “isothiocyanate” describes an-N═C═S group.

The term “nitro” describes an-NO₂ group.

The term “acyl halide” describes a —(C═O)R′″ group wherein R′″ is halide, as defined hereinabove.

The term “azo” or “diazo” describes an-N═NR′ end group or an-N═N— linking group, as these phrases are defined hereinabove, with R′ as defined hereinabove.

The term “peroxo” describes an —O—OR′ end group or an —O—O— linking group, as these phrases are defined hereinabove, with R′ as defined hereinabove.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone.

Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and O-thiocarboxylate.

The term “C-thiocarboxylate” describes a —C(═S)—OR′ end group or a —C(═S)—O— linking group, as these phrases are defined hereinabove, where R′ is as defined herein.

The term “O-thiocarboxylate” describes a —OC(═S)R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone.

Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and O-carbamate.

The term “N-carbamate” describes an R″OC(═O)—NR′— end group or a —OC(═O)—NR′-linking group, as these phrases are defined hereinabove, with R′ and R″ as defined herein.

The term “O-carbamate” describes an —OC(═O)—NR′R″ end group or an —OC(═O)—NR′— linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “carbamate” as used herein encompasses N-carbamate and O-carbamate.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking group, as these phrases are defined hereinabove, with R′ and R″ as defined herein.

The term “N-thiocarbamate” describes an R″OC(═S)NR′— end group or a —OC(═S)NR′-linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a —SC(═S)—NR′R″ end group or a —SC(═S)NR′— linking group, as these phrases are defined hereinabove, with R′ and R″ as defined herein.

The term “N-dithiocarbamate” describes an R″SC(═S)NR′— end group or a —SC(═S)NR′-linking group, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking group, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking group, with R′, R″ and R′″ as defined herein.

The term “amide” as used herein encompasses C-amide and N-amide.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″— end group or a R′C(═O)—N— linking group, as these phrases are defined hereinabove, where R′ and R″ are as defined herein.

An amide can be linear or cyclic. When cyclic, R′ and the carbon atom are linked together to form a ring, in C-amide, and this group is also referred to as lactam. Cyclic amides can function as a linking group, for example, when an atom in the formed ring is linked to another group.

The term “guanyl” describes a R′R″NC(═N)— end group or a —R′NC(═N)— linking group, as these phrases are defined hereinabove, where R′ and R″ are as defined herein.

The term “guanidine” describes a —R′NC(═N)—NR″R′″ end group or a —R′NC(═N)—NR″— linking group, as these phrases are defined hereinabove, where R′, R″ and R′″ are as defined herein.

The term “hydrazine” describes a —NR′—NR″R′″ end group or a —NR′—NR″— linking group, as these phrases are defined hereinabove, with R′, R″, and R′″ as defined herein.

As used herein, the term “hydrazide” describes a —C(═O)—NR′—NR″R′″ end group or a —C(═O)—NR′—NR″— linking group, as these phrases are defined hereinabove, where R′, R″ and R′″ are as defined herein.

As used herein, the term “thiohydrazide” describes a —C(═S)—NR′—NR″R′″ end group or a —C(═S)—NR′—NR″— linking group, as these phrases are defined hereinabove, where R′, R″ and R′″ are as defined herein.

As used herein, the term “alkylene glycol” describes a —O—[(CR′R″)_{z}—O]_{y}-R′″ end group or a —O—[(CR′R″)_{z}O]_{y}— linking group, with R′, R″ and R′″ being as defined herein, and with z being an integer of from 1 to 10, preferably, 2-6, more preferably 2 or 3, and y being an integer of 1 or more. Preferably R′ and R″ are both hydrogen. When z is 2 and y is 1, this group is ethylene glycol. When z is 3 and y is 1, this group is propylene glycol.

When y is greater than 4, the alkylene glycol is referred to herein as poly(alkylene glycol). In some embodiments of the present invention, a poly(alkylene glycol) group or moiety can have from 10 to 200 repeating alkylene glycol units, such that z is 10 to 200, preferably 10-100, more preferably 10-50.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Materials:

Dexamethasone (DEX) was obtained from a commercial vendor.

All chemical reagents were obtained from commercial vendors and used without further purification, unless otherwise indicated.

Peptides were synthesized using an AAPPTEC Focus XC automated synthesizer. Amino acids were obtained from AAPPTEC, ChemPep or NovaBiochem.

Modified 2^{nd} generation Grubbs ruthenium initiator, (IMesH₂)(C₅H₅N)₂(Cl)₂Ru═CHPh, was prepared according to a known procedure [Sanford et al. Organometallics, 2001, 20 (25), 5314-5318].

(N-Benzyl)-5-norborene-exo-2,3-dicarboximide (Nor-Ph) monomer was synthesized according to a known procedure [Ku et al., J. Am. Chem. Soc. 2011, 133, 8392-8395].

All polymerizations were carried out under inert atmosphere in a glove box.

### Analyses:

All polymerizations that were monitored by NMR were ran in J. Young NMR tubes (5 mm diameter) in a glove box under an inert atmosphere (N₂) using DMF-d7 from a sealed vial (Cambridge Isotopes). Other polymerizations were ran in flame dried vials using anhydrous dimethylformamide (DMF) under an inert atmosphere (N₂). Analytical HPLC was performed on a Jupiter Proteo90A Phenomenex column (150×4.60 mm) using a Hitachi-Elite LaChrome L-2130 pump equipped with a UV-Vis detector (Hitachi-Elite LaChrome L-2420). PREP HPLC was performed on Jupiter Proteo90A Phenomenex column (2050×25.0 mm) on a Waters DeltaPrep 300 system. HPLC was ran using water with 0.1% TFA as buffer A, and acetonitrile with 0.1% TFA as buffer B.

Purified peptide monomers were confirmed by analytical HPLC and ESI-MS.

Polymer dispersities and molecular weights were determined by size-exclusion chromatography (SEC-MALS; phenomenex Phenogel 5μ 10, 1 k-75 k, 300×7.80 mm in series with a Phenomex Phenogel 5u 10, 10K-1000K, 300×7.80 mm (0.05 M LiBr in DMF)) using a Shimadzu pump equipped with a multi-angle light scattering detector (DAWNHELIOS: Wyatt Technology) and a refractive index detector (Hitachi L-2490) normalized to a 30,000 MW polystyrene standard.

¹H NMR (400 MHz) spectra were recorded on a Varian Mercury Plus spectrometer. Chemical shifts were reported in ppm relative to the solvent residual proton peaks.

Peptide synthesis: MMP-peptides (D-peptide, L-peptide and random (rac peptide) were synthesized using standard solid phase peptide synthesis (SPPS) using FMOC chemistries on an AAPPTec Focus XC automated synthesizer. Peptides were synthesized using Rink Amide MBHA resin to prepare protecting group-free peptides. In general, the resin was swelled in DMF for 1-2 hours. The resin was then de-protected; 2×10 minutes with 20% 4-methylpiperidine. Following deprotection, the peptide was washed 3×2 minutes with DMF. The first amino acid was activated with HBTU and DIPEA in a ratio of 3:2.9:6 for amino acid:HBTU:DIPEA for 10 minutes, followed by addition to the resin and mixing for 45 minutes. After coupling, the resin was washed 3×2 minutes with DMF, and the process was repeated for each remaining amino acid.

### Animal Studies:

All experiments were conducted in accordance with the guidelines for animal experiments. Three to six months old Female Sprague Dawley rats (approximately 250 grams) were used as model animals in all experiments. The animals were kept under normal conditions including standard food.

Well-established experimental models in rats were employed:

The “induced periapical lesion” model, wherein, following anesthesia, the pulp of the 1st right molar teeth of the rats was surgically exposed and left open to the oral environment to allow infection from the oral environment, and to induce periapical bone loss [Tani-Ishii et al., Oral Microbiol Immunol, 1994, 9, 129-135], was used to assess the development of pulp infection related periapical bone lesion;

The “palatal wound” model, wherein a circular excisional wound, 5 mm in diameter, was created in the center of the palatal mucosa [Kozlovsky et al, J Clin Periodontol, 2007, 34, 164-171], was used for wound healing assessments;

“Ligature” in the gingival sulcus, wherein a bacterial plaque retentive silk ligature was placed in the gingival sulcus around the 1st right molar teeth [Oz and Puleo, J Biomed Biotechnol, 2011, 2011, 754857] to induce periodontitis;

“Tooth extraction” model, wherein the 1st right molar teeth was extracted [Willett et al., J Periodontol, 2017, 88, 799-807], was used to assess alveolar socket healing and regenerative techniques;

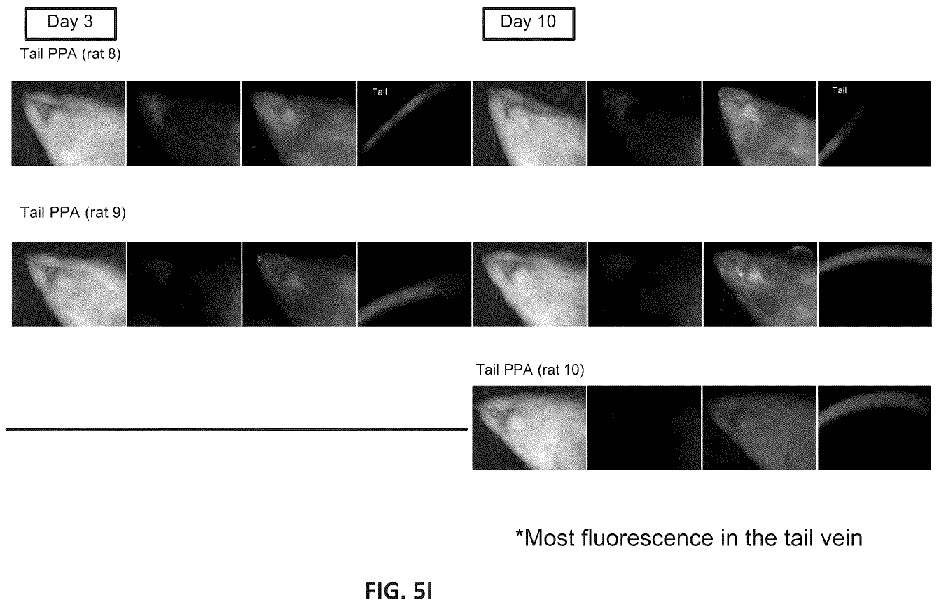

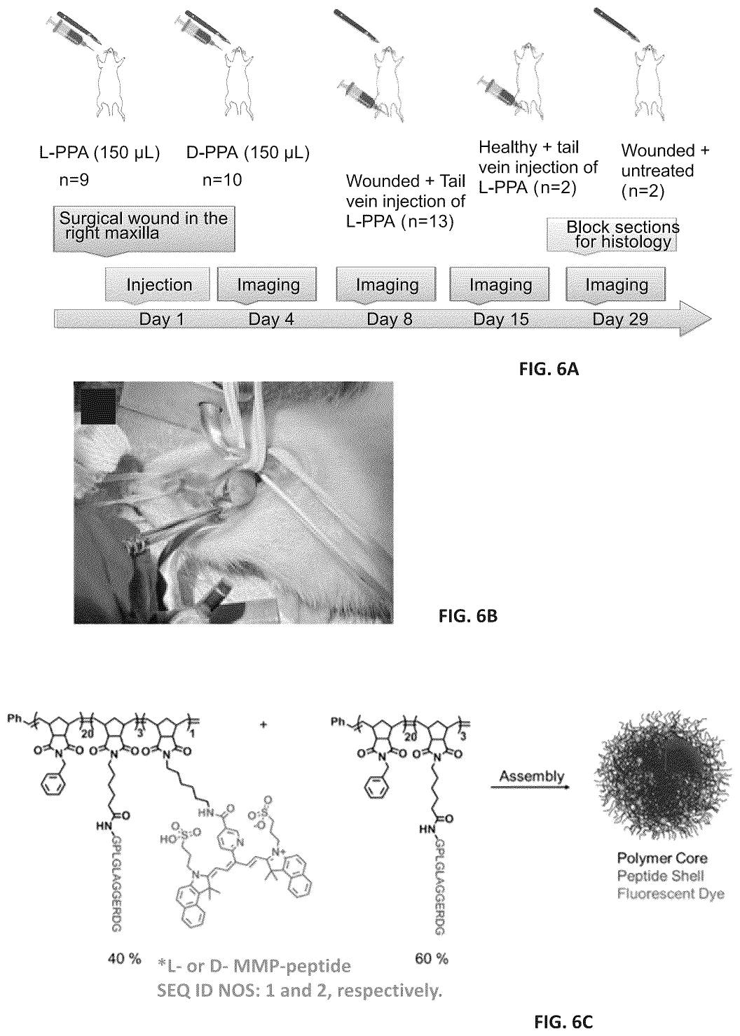

The “surgical wound” (also referred to herein as the “alveolar ridge wound” or “alveolar crest wound”; see, e.g., FIGS. 2A and 6B) model [Maymon-Gil et al., J Periodontal, 2016, 87, 601-9.] was employed for inducing tissue inflammation, wherein a mid-crestal incision was performed on the maxillary alveolar ridge along the existing edentulous area between the first right molar and the incisors;

“Diabetic induction” model [Zoabi et al. 2020, supra]: Type 1-like diabetes was induced through a single intraperitoneal injection of streptozotocin, and the induction of hyperglycemia is confirmed by a blood glucose level that is higher than 270 mg/dL at 7 days post injection;

“Collagen membrane implantation” under scalp of diabetic rats, wherein a para-sagital excisional wound, 10 mm in length, was created in the center of the skin of the calvaria, the periosteum was separated from the skull bone, and a collagen membrane was implanted beneath [Eliezer M, Nemcovsky C, Romanos G, Kozlovsky A, Tal H, Kolerman R, Weinreb M, Moses O. Opposing effects of diabetes and tetracycline on the degradation of collagen membranes in rats. J Periodontol. 2013 April; 84(4):529-34; Tal H, Weinreb M, Shely A, Nemcovsky C E, Moses O. Tetracycline impregnation affects degradation of porcine collagen matrix in healthy and diabetic rats. Clin Oral Investig. 2016 July; 20(6):1237-42; Moses O, Eliezer M, Nemcovsky C, Tal H, Weinreb M. Accelerated degradation of collagen membranes in diabetic rats is associated with increased infiltration of macrophages and blood vessels. Clin Oral Investig. 2016 September; 20(7):1589-96; Eliezer M, Sculean A, Miron R J, Nemcovsky C, Weinberg E, Weinreb M, Zoabi H, Bosshardt D D, Fujioka-Kobayashi M, Moses O. Hyaluronic acid slows down collagen membrane degradation in uncontrolled diabetic rats. J Periodontal Res. 2019 December; 54(6):644-652; and Zoabi H, Nemcovsky C E, Bender O, Moses O, Weinreb M. Accelerated degradation of collagen membranes in type 1 diabetic rats is associated with increased expression and production of several inflammatory molecules. J Periodontol. 2020 October; 91(10):1348-13561.

### In-Vivo Whole Body Assessments:

In order to assess the delivery and accumulation of administered fluorescent-labeled PPAs, fluorescence was detected as described, for example, in Ferber et al., Cancer Lett, 2014, 352, 81-89, using the Maestro noninvasive fluorescence imaging system (CRI Inc., Hopkinton, MA, USA) using deep red filters (excitation in the range of 671-705 nm, emission of 750 nm long pass) to match the Cy5.5 fluorophore excitation and emission spectra. Animals were positioned to capture images of the upper half of the body from ventral views, centered on the maxillary alveolar ridge. The PPA fluorescent signal intensity in each image (e.g., FIGS. 4A and 4B) was then analyzed with the Maestro built-in software (CRI Inc., Hopkinton, MA, USA) after defining the specific regions of interest. The PPA signal intensity per unit area normalized to adjacent background intensity was expressed as relative fluorescence units.

Ex-vivo kinase assay: Immediately following the final in-vivo whole body imaging session, animals were euthanized by CO₂ inhalation. Maxillae were harvested and prepared for ex-vivo maxilla assessment with the Maestro noninvasive fluorescence imaging system, as described above, and histological samples were prepared.

Histological evaluations: Rat maxillae were fixed in 4% paraformaldehyde for 7 days. After decalcification with 10% EDTA, all specimens were embedded in paraffin and sections were cut in a coronal plane from the middle area of the incision, were stained with hematoxylin and eosin and were analyzed.

Transmission electron microscopy (TEM): Nanoparticles were characterized using a Hitachi HD-2300 STEM. Grids were prepared using a 1% uranyl acetate solution.

### Example 1

### Design and Syntheses

Polymer peptide amphiphiles (PPAs) have the potential to solve current problems associated with anti-inflammatory therapeutics by serving as a targeted drug delivery vehicle to prolong the half-life of commercially available therapeutic agents (e.g., dexamethasone).

The present inventors have conceived using peptide-polymer amphiphiles (PPAs; also referred to herein as block copolymers) for forming micellar particles (PPA NPs) that can serve as a targeted drug delivery platform for the treatment of local and systemic inflammations (e.g., oral lesions), as shown in FIGS. 2A-C.

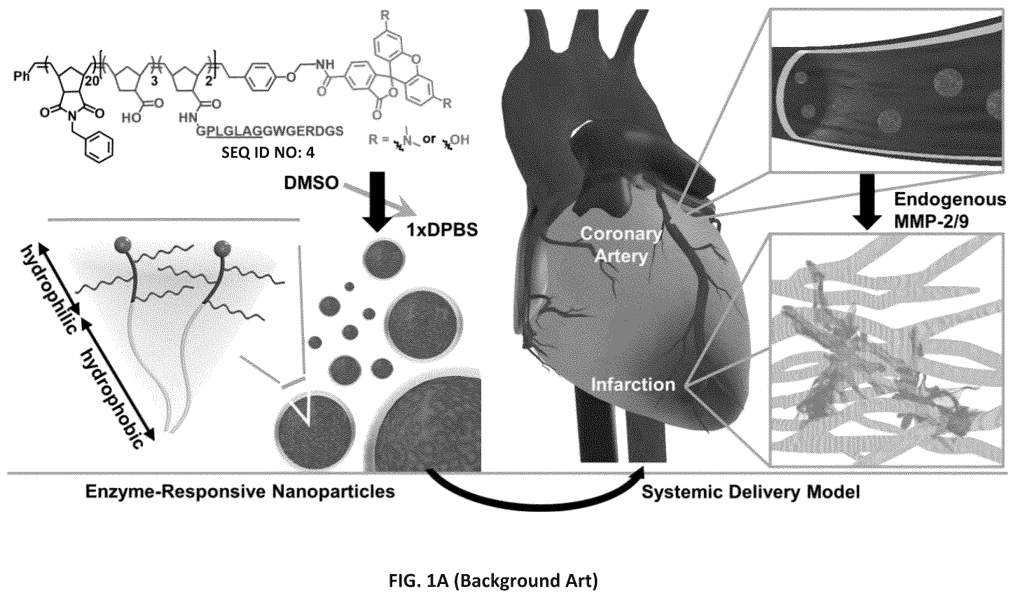

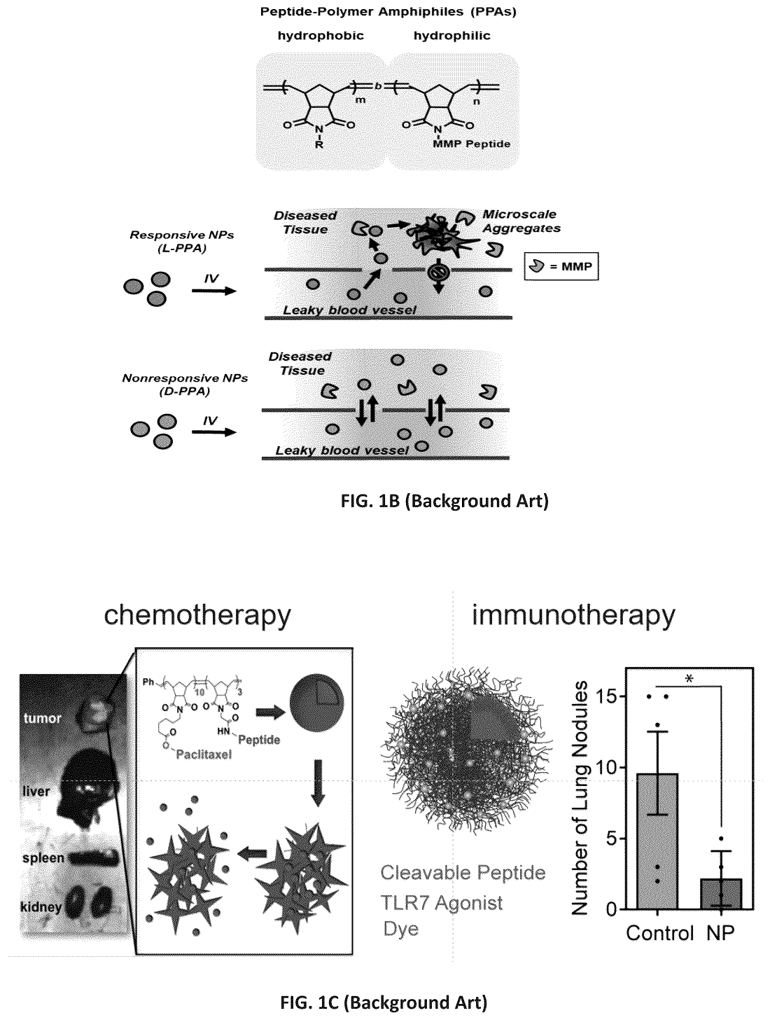

Previously reported PPA NPs (also referred to herein as “micellar particles”) contained a norbornene-based hydrophilic block having MMP-cleavable peptide conjugated to the backbone units (which forms the shell of the NPs), a norbornene-based hydrophobic block substituted with a phenyl moiety as an exemplary hydrophobic moiety (which forms the core of the NPs), and a labeling agent (which is also present at the shell of the NPs), as shown in Background Art FIG. 1A. The MMP-responsive peptide (MMP-cleavable sequence) is GPLOLAQGERDG (SEQ ID NO.: 1), which the MMP-cleavable portion underlined.

Other norbomene-based targeted PPA comprise a therapeutic peptide (e.g., terlipressin), a dye and paclitaxel, as shown in Background Art FIG. 1B were previously disclosed [see, International Patent Application Publication No. WO 2021/030326].

While previously described Rhodamine-labeled PPA NPs are useful for ex-vivo tissue imaging, the present inventors have conceived using the fluorescent Cy5.5 for monitoring labeled PPAs in-vivo.

Norbornene-based PPAs were prepared using a Cy5.5 as an exemplary fluorophoric labeling agent via the synthetic procedure described hereinbelow, so as to track the delivery and accumulation of the PPA NPs by in-vivo and/or ex-vivo fluorescence imaging.

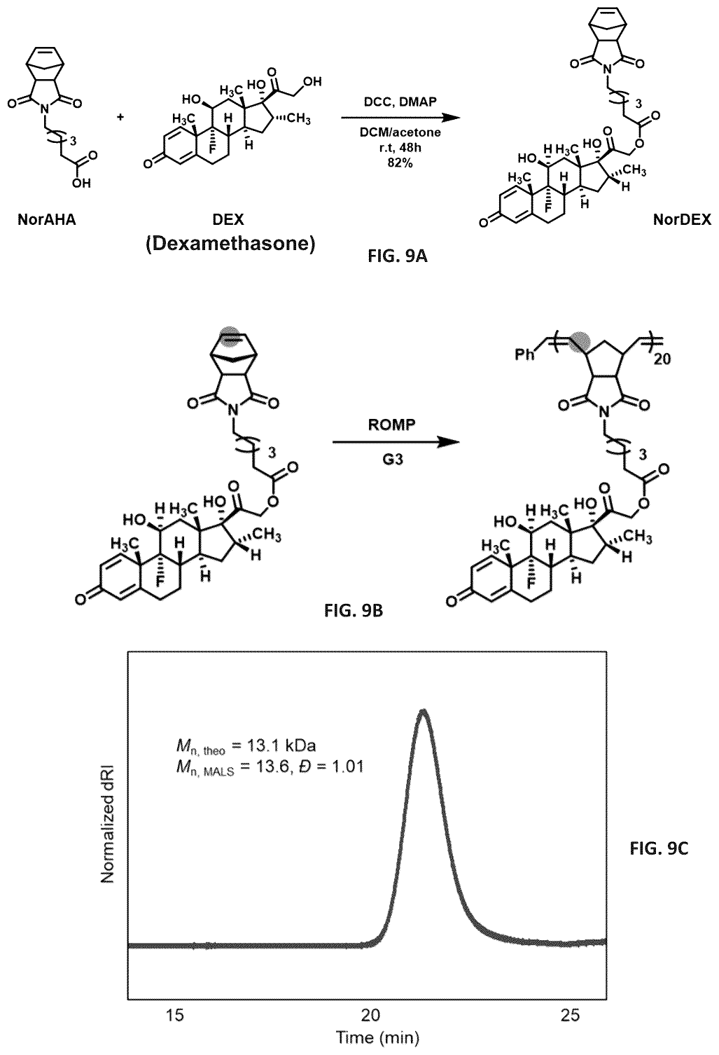

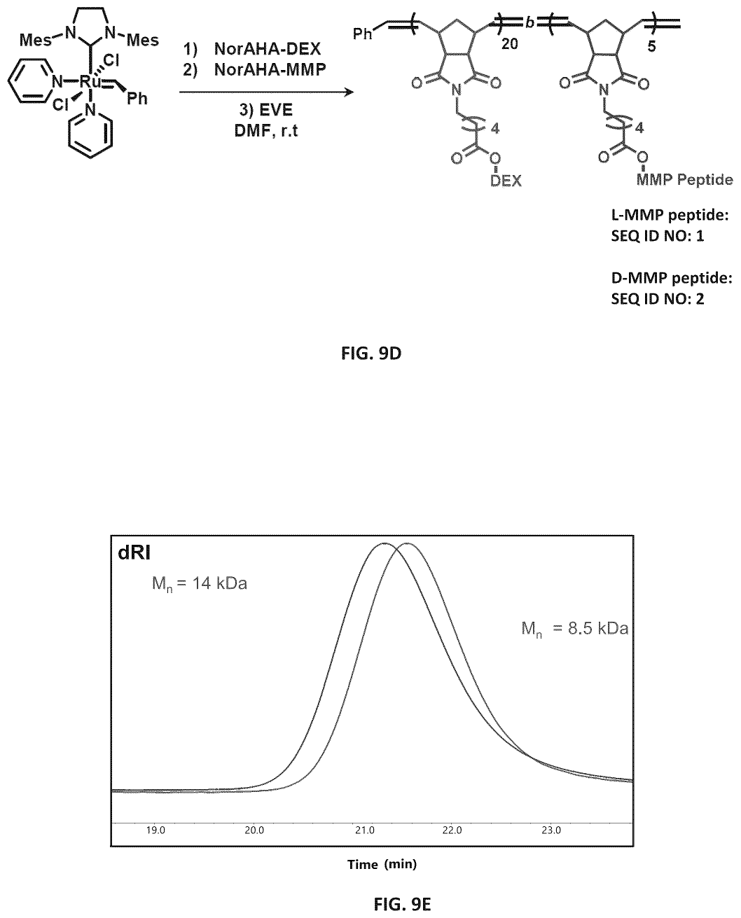

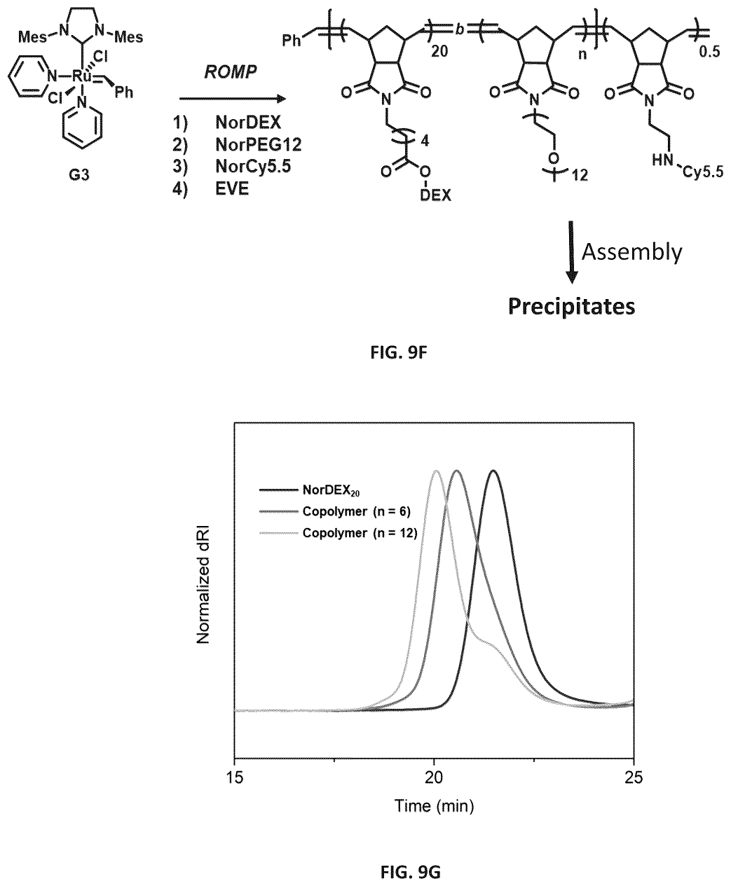

The present inventors have designed and successfully practiced novel stimuli-responsive PPAs, which bear a moiety that is responsive to an inflammation biomarker (e.g., MMP), an anti-inflammatory drug and optionally a suitable labeling agent. Exemplary newly designed norbornene-based MMP-responsive PPAs (block copolymers), as shown in FIGS. 9D and 10A, were prepared based on previously established synthetic procedures [Ungerleider et al., 2017, supra].

### Synthesis of a Drug-Containing ROMP Polymerizable Monomer:

Generally, a drug-containing ROMP polymerizable monomer is prepared by coupling a therapeutic agent (e.g., dexamethasone) to a respective derivative of norbornene dicarboximide that is N-substituted by a group that comprises (e.g., is or terminates by) a reactive group that can be coupled to a chemically compatible group of the therapeutic agent (which can be either intrinsic or generated for the purpose of the coupling). Coupling is typically performed in a suitable organic solvent. The obtained drug-containing monomer is isolated (e.g., by filtration), purified (e.g., precipitated and washed, and/or purified by HPLC) and its chemical structure verified (e.g., by NMR and ESI-MS).

Following is an exemplary representative synthetic protocol of an exemplary dexamethasone-containing norbomene monomer, which is also referred to herein as NorDex (see, FIG. 9A).

Commercially available dexamethasone (DEX) was conjugated to NorAHA through one-step Steglich esterification to afford the NorDEX monomer (see, FIG. 9A). ESI-MS and ¹H NMR confirmed the ester bond was formed on the terminal hydroxyl of dexamethasone (data not shown). No side

To 10 mL acetone, (N-hexanoic acid)-5-norbornene-exo-2,3-dicarboximide (NorAHA; 389 mg, 1.4 mmol, 1.1 equivalents), dicyclohexylcarbodiimide (DCC; 289 mg, 1.4 mmol, 1.1 equivalents) and 4-dimethylaminopyridine (DMAP; 31 mg, 0.25 mmol, 0.2 equivalents) were sequentially added and stirred for 5 minutes. In a separate vial, dexamethasone (DEX; 500 mg, 1.2 mmol, 1.0 equivalents) was dissolved in 10 mL acetone and then added to the reaction mixture. The reaction was stirred for 48 hours at room temperature (r.t), and was thereafter heated to 40° C. for 1 hour to push the conversion. After cooling back to r.t, the insoluble urea by-product was filtered, and the reaction mixture was concentrated under reduced pressure. The obtained crude was purified by column chromatography (60% ethyl acetate (EtOAc) in Hexane as eluent), affording the pure product as a white solid.

¹H NMR confirmed the ester bond was formed through the terminal hydroxyl on DEX. No side products were observed despite the presence of two other free hydroxyls on the fused rings, which is probably due to the steric hindrance.

¹H NMR (400 MHz, DMSO-d₆) δ 7.30 (d, J=10.1 Hz, 1H), 6.31 (t, J=1.8 Hz, 2H), 6.23 (dd, J=10.1, 1.9 Hz, 1H), 6.01 (t, J=1.7 Hz, 1H), 5.40 (dd, J=4.9, 1.4 Hz, 1H), 5.15 (s, 1H), 5.02 (d, J=17.6 Hz, 1H), 4.79 (d, J=17.6 Hz, 1H), 4.19-4.11 (m, 1H), 3.35 (t, J=7.2 Hz, 2H), 3.11 (t, J=1.8 Hz, 2H), 2.87 (ddd, J=11.3, 7.4, 4.1 Hz, 1H), 2.69 (d, J=1.4 Hz, 2H), 2.35 (dt, J=13.8, 7.6 Hz, 4H), 2.25-2.04 (m, 2H), 1.82-1.53 (m, 6H), 1.49 (s, 3H), 1.42-1.19 (m, 4H), 1.19-0.99 (m, 4H), 0.88 (s, 3H), 0.79 (d, J=7.2 Hz, 3H).

ESI-MS: calculated for C₃₇H₄₆FNO₈ [M−H]⁻ 650.32; Found 650.43.

Test polymerization of NorDEX in DMF in the presence of a third generation Grubbs catalyst (G3; (IMesH₂)(C₅H₄NBr)₂(Cl)₂Ru═CHPh) showed good compatibility towards ROMP, as confirmed by complete monomer conversion into a well-defined polymer within 30 minutes following the addition of the catalyst (FIG. 9B).

### Preparation of Dex-Containing ROMP-Polymer:

NorDex was polymerized in the presence of a Grubbs catalyst as described herein, as shown in FIG. 9B. The obtained polymer was characterized using SEC-MALS as described herein and the obtained data is presented in FIG. 9C.

NorDex showed good compatibility towards ROMP, as confirmed by complete monomer conversion into a well-defined polymer within 30 minutes post the catalyst addition (data not shown).

### Preparation of MMP Peptide-Containing ROMP-Polymerizable Monomers:

The present inventors have uncovered that the previously described graft-to approach, where peptides were introduced via post polymerization modification by replacing an activated N-hydroxysuccinimide (NHS) group that substitutes a ROMP building unit, requires dialysis and lyophilization in order to obtain the final PPA, which is time consuming and may contribute to product loss and batch-to-batch variations.

An alternative strategy has therefore been adopted, in which graft-through polymerization is performed with peptide functionalized monomers. To achieve this, the peptide is first modified with a ROMP-polymerizable moiety (e.g., a norbornene monomer), and then directly incorporated into the growing polymer chain. This allows for precise control of the peptide density on the polymer and simplifies the material preparation procedures.

An exemplary MMP peptide-containing norbornene monomer, which is also referred to herein as Nor-MMP or NorAHA-MMP was prepared as follows (See, FIG. 2A):

A selected MMP-responsive peptide was synthesized via SPPS as described in Materials and Methods. With the peptide still on resin, NorAHA (3 equivalents), hexafluorophosphate benzotriazole tetramethyl uronium (HBTU; 3.0 equivalents) and diisopropylethylamine (DIPEA; 6.0 equivalents) in DMF were added. After 24 hours, the reaction solution was drained, and the resin was rinsed with DCM three times. The peptide-containing monomer was cleaved by treating with 88%:5%:5%:2% TFA:TIPS:DTT:H₂O for 3 hours. The resulting solution was vacuum-dried before addition of cold diethyl ether for peptide precipitation. The crude product was dried and purified via a preparation grade HPLC over a gradient of 20-60% Buffer B in 30 minutes. The product eluted out at 16.5 minutes (about 40% B), was collected and dried via lyophilization to afford the pure monomer as a white powder.

ESI-MS: calculated for C₇₇H₁₁₁N₂₁O₂₃[M+H]⁺1687.82; Found 1688.09.

The same approach was used to synthesize a norbornene monomer with D-MMP peptide (NorMMP_{D}) and R-MMP peptide (NorMMP_{R}). For NorMMP_{R}, a 20-40% Buffer B over 30 minutes gradient was used for purification.

ESI-MS: calculated for C₆₇H₉₅N₂₁O₂₆ [M+H]⁺⁼1610.68; Found 1610.98.

L-MMP peptide monomer was synthesized (SEQ ID NO: 1). D-MMP peptide monomer was synthesized in a similar manner (SEQ ID NO: 2), using D-amino acids in the PLGLAG (SEQ ID NO: 16) MMP-cleavable portion.

Herein throughout, “MMP peptide”, which is also referred to herein as MMP-responsive peptide, MMP-cleavable peptide, and like terms, encompasses a peptide that comprises a PLGLAG amino acid sequence (SEQ ID NO: 16). The peptide can comprise additional amino acids, the majority of which are preferably hydrophilic amino acid residues, and is typically of 10-30 or of 10-20 amino acids in length. In some embodiments, an MMP peptide has a glycine residue as the terminal amino acid residue at the attachment point to the ROMP monomer. Unless otherwise indicated, an MMP peptide comprises a PLGLAG amino acid sequence in which all amino acids are L-amino acids (SEQ ID NO: 1) and is referred to herein also as L-MMP and is considered as MMP-cleavable or MMP-responsive. An MMP peptide that comprises a PLGLAG amino acid sequence in which all amino acids are D-amino acids (SEQ ID NO: 2) and is referred to herein also as D-MMP and is considered as MMP-non-cleavable or MMP-non-responsive.

R-MMP represents a random peptide having SEQ ID NO: 17.

### Preparation of PPAs:

Preparation of Dex- and peptide-containing PPAs (Dex-Pep-PPAs) was performed via ROMP, using a ROMP Grubbs type catalyst, using the NorDex and NorPep (norbornene as described herein having a MMP peptide as described herein attached thereto) monomers as described herein. The catalyst was quenched with a backbone terminating group (e.g., EVE).

An exemplary synthesis, of a ROMP-polymerized Dex and MMP peptide-containing PPA, which is referred to herein as (NorDEX)₂₀-(Nor-Pep)₅-PPA, is presented in FIG. 9D.

In a search for a non-responsive control for animal studies, the present inventors have designed a PPA in which the peptide moiety is replaced with a poly(ethylene glycol) (PEG12, Mₐ˜500) which is hydrophilic and inert to MMP. Two PEG12 functionalized block copolymers with different DP of PEG12 were synthesized, giving a hydrophilic weight percentage of 0.25 (DP of 6) and 0.5 (DP of 12) (see, FIG. 9F). Significant precipitation was observed when PEG12-polymer amphiphiles were dialyzed into DPBS for self-assembly. This indicates that the PEG brush interacts differently with the buffer solution and hydrophobic polynorbornene, and thus cannot provide enough surface curvature to stabilize the nanoparticle structure. SEC traces of the PEG12 functionalized polymers are shown in FIG. 9G.

A new random peptide sequence was designed by replacing the MMP recognizable amino acid sequence PLGLAG (SEQ ID NO: 16) into GSGSGS (SEQ ID NO: 20). The resulting peptide maintains good water solubility and has the same net charge of −1 at pH=7 as the original MMP peptide. Hence, it should provide similar hydrophilic volume fraction that allows PPA self-assembly into micellar nanoparticle.

PPAs made of block(s) of NorDex building units and block(s) of variable NorMMP building units having L-MMP, D-MMP or R-MMP peptides as pendant groups, referred to as PPA_{L}, PPA_{D}, PPA_{R} respectively, were prepared as shown in FIG. 10A, using the following procedure.

DMF was freeze-pump-thawed three cycles to remove air. All the monomers and the third generation Grubbs catalyst G3 (IMesH₂)(C₅H₄NBr)₂(Cl)₂Ru═CHPh were weighed separately in vials charged with stirring bars. The monomers and DMF were then loaded into the glovebox under N₂. To G3 (8.2 mg, 1.0 equivalent) in DMF, NorDEX (147 mg, 20.0 equivalents) in DMF was added to afford the NorDEX₂₀ block. After 45 minutes, an aliquot (˜10 μL) was removed and terminated with EVE for SEC-MALS analysis. The reaction mixture was then evenly divided into three vials, and one of the NorMMP monomers with L, D, or R peptides (31 mg, 5.0 equivalents; SEQ IDs: 1, 2, and 17, respectively) was added to each vial to form the second block. After 3 hours, a half of the solution in each vial was moved out of the glovebox and terminated with EVE. The molecular weights of the block copolymers were analyzed by SEC-MALS (see, Table 2 hereinbelow). For the other half of the reaction solution, a Cy5.5 labeled norbornene monomer (NorCy5.5, 1.0 equivalent) was added and stirred for 2 hours before catalyst quenching with EVE. The resulting peptide polymer amphiphiles were precipitated in cold diethyl ether and dried in vacuum to afford PPA_{L}, PPA_{D} and PPA_{R}.

### Ppa Nps Formation:

PPA NPs are typically prepared using a solvent exchange method (e.g., from DMSO into DPBS), as previously described.

In an exemplary procedure, a PPA as described herein is dissolved at 3 mg/mL in DMSO. 1× Dulbecco's phosphate-buffered saline (DPBS, without Ca²⁺ and Mg²⁺) is added via a syringe pump at a rate of 100 IL/hour until reaching 30% DPBS in DMSO (v/v). The solution is stirred overnight and transferred into SnakeSkin™ Dialysis Tubing (10K MWCO) to dialyze against DPBS for 48 hours with three buffer changes. The resulting solution is filtered through a 0.22 μm PES filter to remove bacteria and any large aggregates. The polymer concentration of the filtered solution is confirmed by UV absorbance from Cy5.5, if present. The nanoparticle solution is concentrated by spin centrifugation.

Since the D-MMP peptide can still be recognized and cleaved by MMP in-vivo, a non-responsive control was required for animal studies.

In a preliminary attempt, the peptide sequence was replaced with a poly(ethylene glycol) (PEG12, Mₐ˜500) which is hydrophilic and inert to MMP. Two PEG12 functionalized block copolymers with different DP of PEG12 were synthesized (FIG. 10B). A significant precipitation was observed when PEG12-PPAs were dialyzed into DPBS for self-assembly.

The SEC-MALS characterization data (also shown in FIG. 10C) of PEG12-comprising DEX-PPAs are presented in Table 1 herein.

TABLE 1

| | | NorDEX₂₀-b- | NorDEX₂₀-b- |
|---|---|---|---|
| | NorDEX₂₀ | NorPEG12₆ | NorPEG12₁₂ |
| M_{n, theo} (kDa) ^{a} | 13.0 | 17.2 | 21.5 |
| M_{n, MALS} (kDa) ^{b} | 9.0 | 11.5 | 14.7 |
| Ð | 1.03 | 1.03 | 1.03 |

^{a} Theoretical molecular weight M_{n, theo} = Σ DPₘₒₙₒₘₑᵣ × MW_{monomer.}.
^{b} Molecular weight and dispersity were determined by SEC-MALS with a dn/dc of 0.179 mL/g in DMF with 0.1M LiBr.

Without being bound by any particular theory, it can be assumed that the PEG brush interacts differently with the buffer solution and hydrophobic polynorbornene, and thus cannot provide enough surface curvature to stabilize the nanoparticle structure.

Consequently, new random peptide sequences (i.e., having GGSGSGSGWGERDGS, SEQ ID NO: 17) were designed by replacing the MMP recognition amino acid sequence PLGLAG (SEQ ID NO: 16) into a random GSGSGS (SEQ ID NO: 20).

The resulting peptide maintains good water solubility and has a similar same net charge as the original MMP peptide (˜1 at pH=7). Hence, it was expected that similar hydrophilic volume fraction will allow PPA self-assembly into micellar nanoparticle. Following the preparation of peptide functionalized norbornene monomers, an MMP-9 cleavage assay was performed setup, as described in the Materials and Methods section hereinabove.

### Characterization:

Stock solution of NorMMP peptide (510 μM) was prepared in DPBS buffer. To an Eppendorf tube, 1.5 μL MMP-9 stock (38.46 μM) and 105 μM of NorMMP stock was added, giving a final concentration of 0.5 μM MMP-9: 500 μM NorMMP. The solution was incubated at 37° C. for 24 hours to promote peptide cleavage. To prepare the HPLC sample, 50 μL of the reaction mixture was added to 150 μL of DPBS buffer. 40 μL of resulting solution was injected into HPLC for analysis. For NorMMP_{L}, NorMMP_{D} and the cleavable sequence (LAGGWGERDGS), a gradient of 20-60% Buffer B over 30 minutes was used. For the NorMMP_{R}, a gradient of 20-40% Buffer B in 30 minutes was used.

The nanoparticle cleavage experiment was performed similarly as described in Materials and Methods section, using a ratio of 1 μM thermolysin:100 μM polymer for 24 hours incubation at 37° C.

### MMP-9 Cleavage Assay:

Three test groups, including the norbornenes functionalized with random peptide (NorMMP_{R}), D- and L-MMP peptide (NorMMP_{D} and NorMMP_{L}), and one control group with the cleaved peptide sequence LAGGWGERDGS were setup. Post 24 hours of incubation with MMP-9 at 37° C., NorMMP_{L} was completely cleaved, while the NorMMP_{R} and NorMMP_{D} remained intact.

As can be seen by the HPLC traces (FIG. 10C), both the random peptide (NorMMP_{R}) and NorMMP_{D} remained intact following MMP-9 treatment, while responsive NorMMP_{L} was completely cleaved by the enzyme, which is evidenced by the shift of HPLC signal (FIG. 10C).

Consequently, the fabrication of both the D-MMP and the random R-peptide (R-Pep, or denoted by “R”) containing NPs was performed in order to serve as non-responsive controls in the animal study.

DEX incorporated PPAs that contain L-, D-MMP peptide or random peptide (PPA_{L}, PPA_{D}, and PPA_{R}) were synthesized by graft-through ROMP (FIG. 10A). The step growth of polymer molecular weights was confirmed by SEC-MALS (FIG. 10B, and Table 2 hereinbelow).

TEM analysis confirmed that all the PPAs assembled into spherical micelles with a 20 nm diameter (FIG. 11D, top panel). Upon thermolysin treatment, the nanoparticles with L-MMP peptide (NP_{L}) underwent cleavage induced aggregation, while NP_{D} and NP_{R} maintained their nanoparticle morphology (FIG. 11D, bottom panel).

MMP-responsiveness of L-PPA NPs on MMP was compared to that of D-PPA NPs, in the presence and absence of a thermolysis enzyme. The TEM images are shown in FIGS. 12A-D.

Table 2 presents SEC-MALS characterization of DEX incorporated PPAs (as shown in FIG. 10B).

TABLE 2

| | NorDEX₂₀ | PPA_{L} | PPA_{D} | PPA_{R} |
|---|---|---|---|---|
| Mₙ^{a}, ₜₕₑₒ (kDa) | 13.0 | 21.5 | 21.5 | 21.0 |
| Mₙ^{a}, _{MALS} (kDa) | 10.8 | 16.2 | 16.4 | 18.3 |
| Ð | 1.02 | 1.02 | 1.02 | 1.02 |

^{a}Molecular weight and dispersity were determined as described in Table 1. The PPAs without Cy5.5 dye were used for the SEC-MALS measurement.

### Example 2

### Accumulation and Efficacy Studies

In order to assess the administration and delivery of the norbornene-based PPAs, PPAs without an anti-inflammatory agent (see, FIG. 2A), comprising a block having 40% a polymer having an MMP responsive peptide and a Cy5.5 fluorescent dye conjugated thereto, and 60% an unlabeled polymer (as in FIG. 2A), were initially prepared.

The syntheses of Block and Blend copolymers (e.g., (Nor-Ph)₂ₒ(Nor-Pep)₃(Nor-Cy5.5)-PPA, shown in FIG. 2A) are described, for example, in Ungerleider et al. 2017, supra (see, page S5, therein).

The experimental “surgical wound” model, as described in the “Material and Methods” section hereinabove, was employed for inducing tissue inflammation. Untreated healthy rats served as control. The contra-lateral maxillary alveolar ridge along the existing edentulous area between the first left molar and the incisors served as a negative control.

PPAs (50% labeled with Cy5.5) were dissolved at 3 mg/mL in DMSO. 1× Dulbecco's phosphate-buffered saline (DPBS, without Ca²⁺ and Mg²⁺) was added via a syringe pump at a rate of 100 μL/hour until reaching 30% DPBS in DMSO (v/v). The solution stirred overnight and transferred into SnakeSkin™ Dialysis Tubing (10K MWCO) to dialyze against DPBS for 48 hours with three buffer changes. The resulting solution was filtered through a 0.22 μm PES filter to remove bacteria and any large aggregates. The polymer concentration of the filtered solution was confirmed by UV absorbance from Cy5.5 (data not shown). The nanoparticles solution was concentrated by spin centrifugation to give 300 μM regarding the polymer and 150 μM regarding Cy5.5 dye.

For fluorescent nanoparticles, a block copolymer having 60% (mol) of a block without Cy5.5 and 40% (mol) of a block containing the Cy5.5 were dissolved together in DMSO, as described in Battistella et al. 2019, supra (see, page 3 of the supplementary information, therein; and FIG. 2A herein).

Experiment timeline was as follows: at day 1, a surgical wound was performed as described hereinabove; the rats were injected at day 2; in-vivo whole body assessments were performed by fluorescent imaging at days 3 and 10; and block sections were isolated for histology at day 15.

Two different methods of administering the PPAs were assessed:
(i) local administration (150 μL PPA NPs solution) in the right alveolar ridge; or
(ii) IV injection (1 mL PPA NPs solution) in the tail vein of the nanoparticles.

Both doses were administered from a 200 μM PPA NPs solution. Solutions were prepared in phosphate-buffered saline and sterilized through sterile filters of pore size 0.2 μm prior the injection.

An additional control group consisted of rats injected with saline and with the Cy5.5 fluorophore alone, matching the different administration routes.

In-vivo whole body assessments results are presented in FIGS. 4A-P.

While rats treated by local administration of PPA NPs (FIGS. 4C, 4D, 4K, and 4O) exhibited a strong fluorescence signal along the alveolar ridge, rats treated with IV administration of the PPA NPs (FIGS. 4D, 4G. 4L, and 4P) did not display a signal that was distinguishable from the background fluorescence, similarly to the rats in the control groups (FIGS. 4A, 4B, 4E, 4F, 4I, 4J, 4M, and 4N). Both the locally and IV administrated PPA NPs rat groups exhibited a strong fluorescence signal compared to the background fluorescence of the control rats. However, following IV administration, the signal was diffused in the oral cavity whereas local administration led to a concentration of the signal along the alveolar ridge.

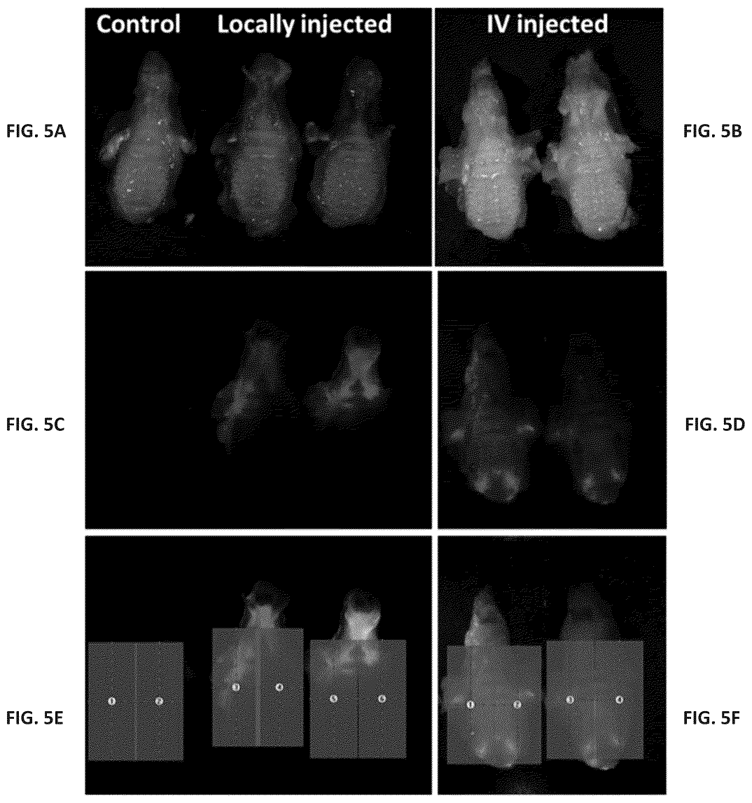

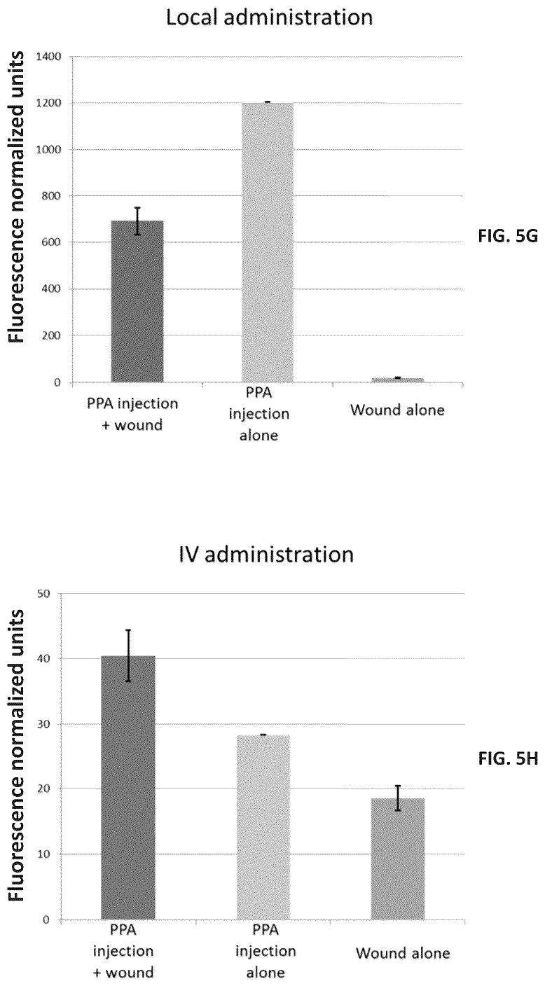

The signal intensity analysis of fluorescent PPA NPs, described hereinabove and presented in FIGS. 5A-F and the quantitative analyses corroborate the imaging data (FIGS. 4A-P) as both the local (FIG. 5G) and IV (FIG. 5H) PPA NPs administration display significantly higher fluorescence levels than the control untreated rats.

Without being bound to any particular theory, it is assumed that the injection itself may have wounded the rats, thus yielding an inflammatory process that could have led to PPA aggregation.

As can be seen in FIG. 5I, signal intensity analysis of fluorescent PPA NPs following tail IV-injection, indicated that most fluorescence was detected mainly in the tail vein. At the 10^{th} day of the study, fluorescence substantially decreased in the maxilla, while the tail vein exhibited strong fluorescent signal.

Overall, the obtained data indicate that the periodontal flap model is a suitable model for evaluating the activity of the exemplary PPA NPs in treating an oral wound with acute inflammation. The tracking of PPA NPs by fluorescence is possible both by in-vivo and ex-vivo fluorescence imaging, with the latter being somewhat advantageous.

To conclude, the delivery and accumulation of the exemplary fluorescent-labeled PPA NPs in an inflamed tissue was successfully determined.

### Accumulation of Targeted PPA Nanoparticles:

The accumulation of injected PPA NPs comprising L-MMP peptide (MMP-responsive, comprising L-amino acid MMP sequence SEQ ID NO: 1) following alveolar crest wound in rats, was assessed and compared to that of control PPA NPs comprising D-MMP peptide (MMP-non-responsive, comprising D-amino acid MMP sequence SEQ ID NO.: 2) [Chien et al., Adv Mater, 2013, 25, 3599-3604].

The L-MMP- and D-MMP-containing NPs are being referred to herein as “L-PPA” and “D-PPA”, respectively or as PPA_{L} and PPA_{D}, respectively. The NPs compose functional norbornene-based monomers containing a hydrophobic phenyl moiety, a Cy5.5 fluorophore and the L-MMP or D-MMP peptide, as described herein (FIG. 6A).

Experiment timeline was as follows: at day 1, a surgical wound in the right maxilla was performed (FIGS. 6B and 6C), as described hereinabove, shortly after the rats were injected; in-vivo whole body assessments were performed by fluorescent imaging at days 4, 8, 15, and 29; and at day 29, block sections were isolated for histology.

L-PPA and D-PPA were locally administered via buccal injection at the previously established dosage of 200 μL from a 200 μM solution as described hereinabove. Wounded untreated rats served as control.

In fluorescence imaging, both the L-PPA (PPA) and D-PPA administrated rats exhibited a strong fluorescence signal (FIG. 7M). Four weeks after local administration, the ex-vivo isolated maxilla fluorescence signal was twice stronger in animals delivered with L-PPA as compared to the control D-PPA (FIG. 7N). As expected, this ratio decreases with time but is still significant after 4 weeks (see imaging in FIG. 7N), suggesting a prolonged retention of the nanoparticles.

The normalized fluorescence signal are presented in FIG. 7O. Although the in-vivo whole body imaging data regarding D-PPA was inconclusive, L-PPA-injected rats clearly showed preferred accumulation in the wounded (right) maxilla compared to the unwounded (left) maxilla. The whole body analyses of wounded rats treated with L-PPA and D-PPA are presented in FIGS. 7A-L (local injection) and FIGS. 7P and 7Q (administered to tail vein) [Schiffiann et al., “Enzyme-Responsive Nanoparticles for Targeted Drug Delivery to Inflamed Oral Tissues”, scientific poster (supra).

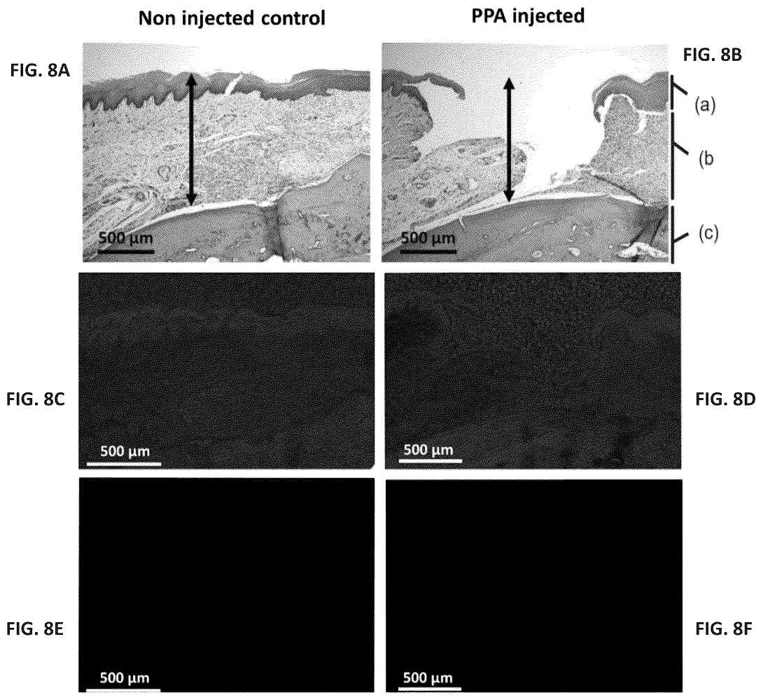

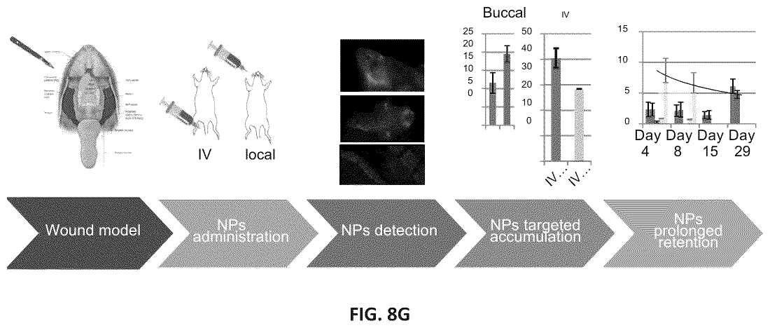

The histological processing protocol was adapted for fluorescence measurements of maxillary tissues sections. The data, obtained at day 10 of the experiment, are presented in FIGS. 8A and 8B. The maxilla and surrounding tissues were retrieved, fixed in 4% neutral buffered formalin, decalcified for 10 weeks in a 10% ethylenediaminetetraacetic acid (EDTA, pH 7.3) solution, washed, dehydrated in ethanol and xylene, and embedded in paraffin. Sagittal 5-μm sections were made, and stained with hematoxylin and eosin (H&E) for histological analysis.

A large epithelial gap was observed in the maxilla of rats that underwent a surgical wound (see FIGS. 8A and 8B, indicating damage in the oral epithelium and lamina propria connective tissue). This observations confirms the induction of an extensive and long lasting inflammatory process. While a broad, background fluorescent signal was observed during imaging unstained sections with a fluorescence microscope (FIGS. 8C and 8D), there was no fluorescence signal in the sections imaged with a confocal microscope tuned for the Cy5.5 fluorophore specific excitation and emission (FIGS. 8E and 8F). This suggests that the fluorescent nanoparticles are not retained intact within the tissue during the histological preparation process.

To conclude, it was established that the inflammatory process is the trigger for the PPA NPs targeted delivery and accumulation in an inflamed oral tissue.

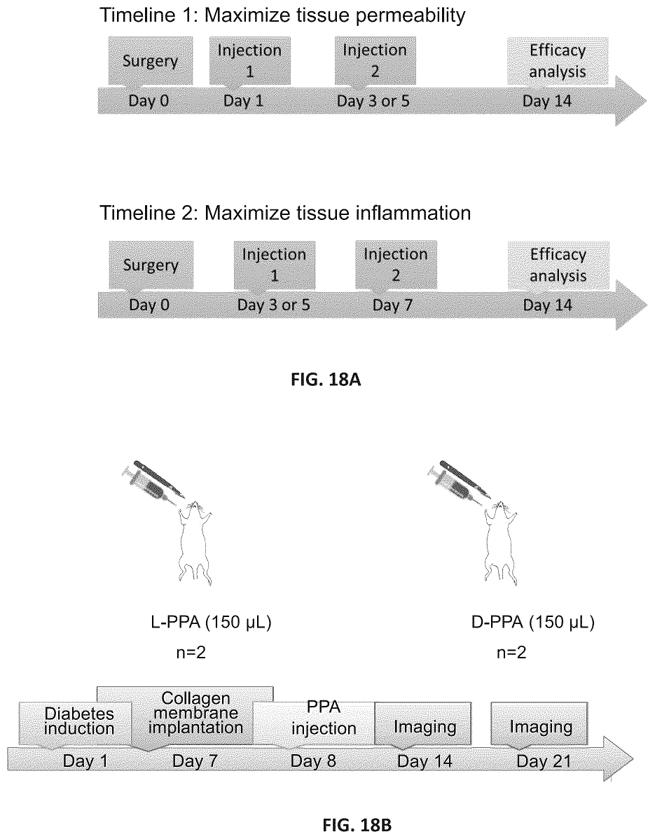

Additional study protocols are presented in FIG. 18A.

### Example 3

### The Effect of MMP-Targeted PPA NPs on Chronic Inflammation

In order to examine whether DEX-containing nanoparticles can respond to systemic inflammation and elicit an anti-inflammatory function in-vivo, the materials were injected adjacent to a wound implanted with a collagen membrane in diabetic rats.

Diabetes mellitus (MD), which is characterized by hyperglycemia, is known to induce exaggerated and chronic inflammation, wherein collagenolytic enzymes, including MMPs, are overexpressed. It was previously shown that DM induction in rats resulted in accelerated degradation of collagen membranes implanted subcutaneously by virtue of increased inflammatory cells infiltrate in and around the membranes. This provides a quantitative approach to evaluate the degree of inflammation based on the histology and residual amount of implanted collagen membrane. If DEX-containing nanoparticles possess an anti-inflammatory effect in-vivo, it is expected to slow down the collagen degradation, leading to a thicker membrane with less cellular infiltration as compared to the ones in untreated diabetic rats.

A schematic illustration of the study protocol is presented in FIG. 18B.

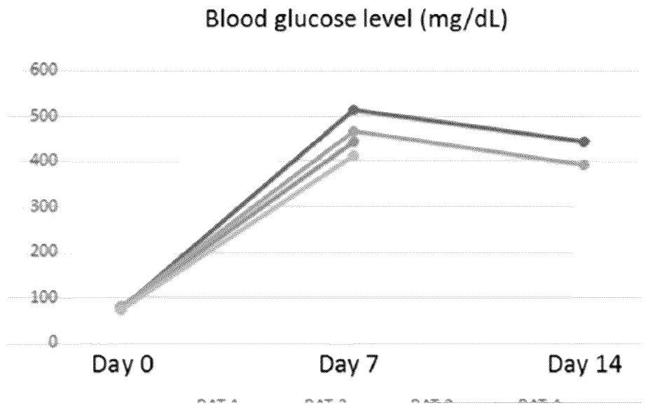

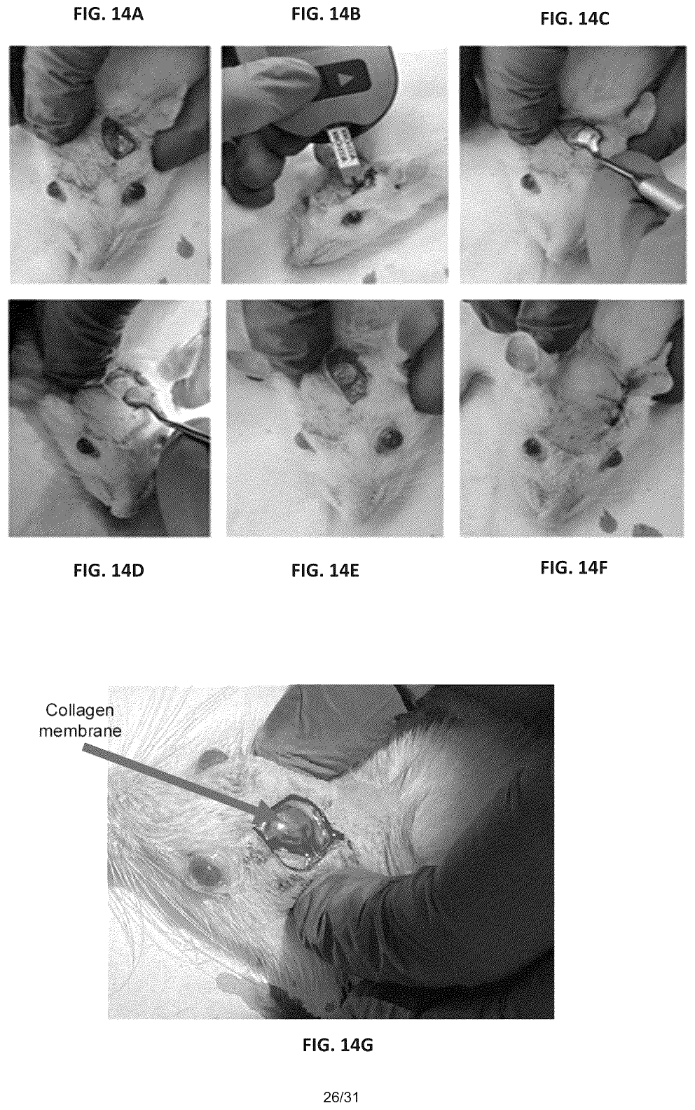

Type 1-like diabetes was induced as described in the Materials and Methods section, and the induction of hyperglycemia was confirmed by a blood glucose level higher than 270 mg/dL 7 days post injection (see, FIGS. 13A and 13B). The biotin-labeled collagen membrane was then implanted under the scalp, as presented in FIGS. 14A-G.

At day 8, the rats were randomly divided into five groups to receive one single dosage of: i) NP_{L}, ii) NP_{D}, iii) NP_{R}, iv) free DEX, or v) no injection, through a subcutaneous injection posterior to the ear (n=12 for each treatment group and n=3 for the no injection control group). For each treated group, 260 μg of DEX (either free-form or conjugated to the polymer) was used to provide a 1 mg/mL dosage similar to the literature value. Thus, the PPA NPs injection contained 250 μM polymer with 50% (125 μM) Cy 5.5 labeling, for each 200 μL injection.

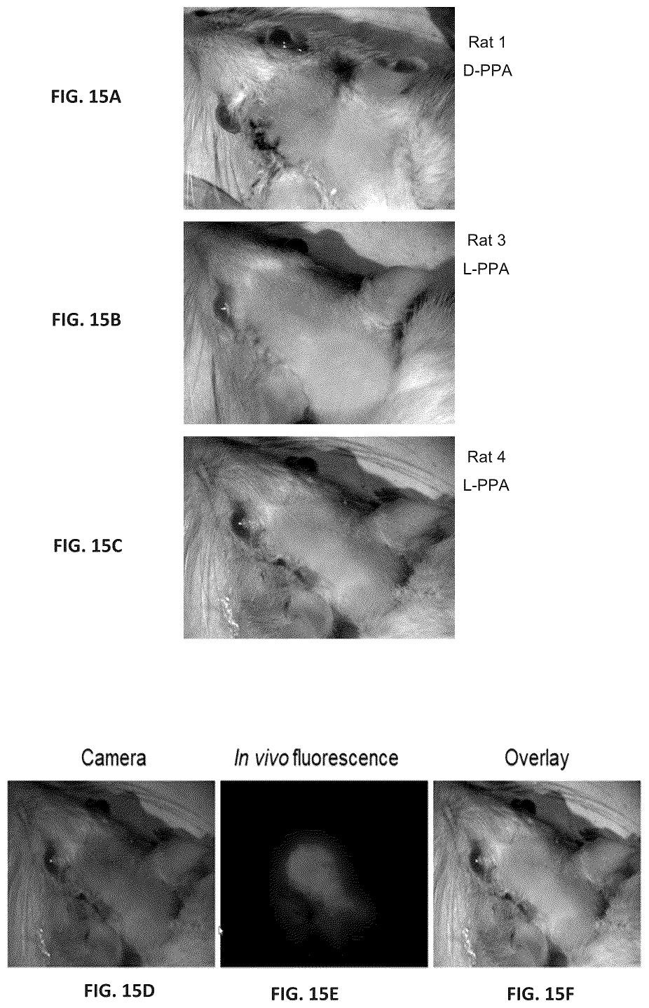

At 7 days post-injection (day 14 of the total protocol), a strong fluorescence was observed around the implanted membrane instead of at the site of injection for all the rats that received nanoparticle treatment. This confirms targeted accumulation of the polymeric material at the site of inflammation (FIG. 15A-F). At both 7- and 14-days post injection (day 14 and day 21), the in-vivo fluorescence intensity from the NP_{L} was significantly higher than the NP_{D} and NP_{R} controls (FIGS. 16A and 16B). The order of fluorescence intensity NP_{L}>NP_{D}>NP_{R} correlated well with the MMP responsiveness of the incorporated peptides. In comparison, no significant difference in the intensity across all three groups were observed during ex-vivo imaging, partly because of a high variance between samples (FIG. 16C).

Two weeks following injection, the rats were sacrificed with carbon dioxide overdose, and the skull together with the over lining periosteum and the membrane were harvested.

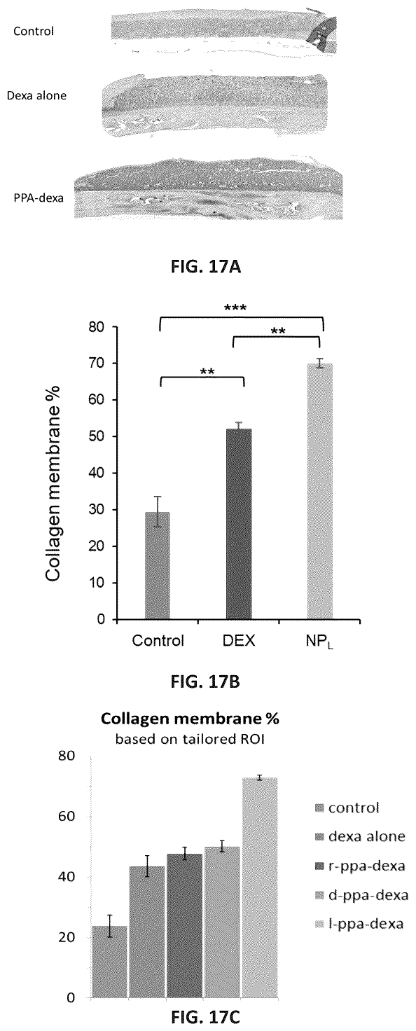

The harvested skull together with the over lining periosteum and the membrane were subjected to the following processing: tissues from 10 rats per group were fixed with paraformaldehyde, and processed for decalcified tissue histology, embedded in paraffin. Sections were stained with HRP-Avidin for measurements of membrane thickness and residual collagen (FIG. 17A).

Histologic analyses show that the remaining collagen membrane area post enzyme-responsive nanoparticles treatment was significantly larger compared to the free-form dexamethasone-treated group.

It can be concluded that incorporation of dexamethasone into the enzyme responsive nanoparticles improves its therapeutic efficacy of reducing inflammation. Without being bound to any particular theory, it is assumed that these targeted DEX-PPAs act by increasing local drug concentration which prolongs its circulation half-life.

Through staining and histology analysis, significantly less voids in the residual collagen membranes were observed in NP_{L} treated rats as compared to the ones from free-form dexamethasone and no injection groups. In particular, the membrane area post NP_{L} treatment was about 20% higher than the free dexamethasone group and about 40% higher than the no treatment group (FIGS. 17B and 17C). These preliminary results suggest that by incorporating dexamethasone into the enzyme responsive nanoparticles, its therapeutic efficacy in reducing inflammation can be improved, potentially by increasing local drug concentration and prolonging its circulation half-life. Characterization and quantification of cell infiltration in the collagen membrane can be performed to further evaluate the degree of inflammation. These include, for example, monitoring mRNA levels of various pro-inflammatory molecules by RT-PCR, and assessing protein amount of inflammatory molecules using western blot.

### Example 4

### Intermediate Concluding Remarks

The use of enzyme-responsive nanoparticles as targeted anti-inflammatory drug delivery method was demonstrated for the successful treatment of inflammatory wounds in hyper and norm glycemic individuals.

The presented data show that following local or intravenous injection, the drug-loaded, PPA-based nanoparticles can reach the inflamed tissue and respond to the overexpressed matrix metalloproteinases (MMPs) to form micro-scale aggregates. The size and morphological transition prevent the material from leaking out of the diseased site and allow the drug to be gradually released through enzymolysis and hydrolysis.

The presented in-vivo results showed that the responsive PPA had prolonged retention (up to 30 days) at the inflamed tissue following administration, while the small molecule drug is typically cleared within 24 hours. Since MMP overexpression has been involved in various inflammatory diseases, these data show that the PPAs of the present embodiments present a generalizable platform for drug delivery to treat local and systemic inflammation.

The enzyme responsiveness of the PPAs allows active targeting and selective accumulation of therapeutics in inflamed tissue to decrease off-target effects associated with conventional systemic administration. The disclosed methodology can therefore be used to enhance the efficacy and maximum tolerated dosage of the drug while reducing the frequency of dosing and side effects.

Thus, the PPA system presents a facile and generalizable approach for the on-demand delivery of different therapeutics.

Other anti-inflammatory drugs-containing PPAs can be prepared similarly to the Dex-containing PPAs from a corresponding anti-inflammatory drug-containing norbornene-based monomer, prepared essentially as described herein, via ROMP as described herein.

Similarly, labeled anti-inflammatory drugs-containing PPAs are prepared as exemplified herein for Cy5.5-labeled Dex-containing PPAs, and their accumulation in the inflamed oral tissues and the therapeutic effect of the drugs on the diseased tissues assessed, essentially as described herein.

Alternatively, targeted drug-containing PPAs can be prepared by conjugating other drugs to ROMP-polymerized backbone units and use such monomers as building blocks for preparing respective targeted PPAs. Such drugs may include antibiotics (e.g., Tetracycline); novel antibacterial therapies (e.g., anti-biofilm factors); and various regeneration promoting factors (e.g., fibroblast growth factor (FGF), platelet-derived growth factor (PDGF), vascular endothelial growth factor (VEGF), and bone morphogenic proteins (BMPs)). Therapeutic peptides and oligonucleotides can be similarly used. The accumulation of the drug-comprising NPs in the tissues and the therapeutic effects of the drugs on the diseased tissues is assessed, essentially as described herein.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating chronic inflammation in a subject in need thereof, the method comprising administering to the subject a micellar particle comprising a plurality of block copolymers, at least a portion, or each, of said block copolymers comprising at least one first block composed of a first plurality of backbone units covalently linked to one another and at least one second block of a second plurality of backbone units covalently linked to one another,
wherein:
the first block comprises at least one anti-inflammatory agent covalently attached, directly or via a linking moiety or group, to at least a portion of the first plurality of backbone units composing said first block; and
the second block comprises at least one hydrophilic moiety covalently attached to at least a portion of the second plurality of backbone units composing said second block, said at least one hydrophilic moiety comprises an amino acid sequence that is capable of interacting with an inflammatory biomarker,
wherein the micellar particle comprises a core comprising said first block and a hydrophilic shell comprising said second block.

2. The method of claim 1, wherein said hydrophilic moiety comprises an inflammatory protease-cleavable amino acid sequence.

3. The method of claim 1, wherein said block copolymer comprises at least two of said first blocks, wherein said at least one anti-inflammatory agent is the same or different in each of said at least two first blocks.

4. The method of claim 1, wherein said first block comprises a first anti-inflammatory drug attached to a first portion of backbone units in said first block and a second anti-inflammatory drug attached to a second portion of backbone units in said first block, wherein said first and second anti-inflammatory agents are different from one another.

5. The method of claim 1, wherein said block copolymer comprises at least two of said second blocks, wherein said at least one hydrophilic moiety is the same or different in each of said at least two second blocks.

6. The method of claim 1, wherein said second block comprises a first hydrophilic moiety attached to a first portion of backbone units in said second block and a second hydrophilic moiety attached to a second portion of backbone units in said second block, wherein said first and second hydrophilic moieties are different from one another.

7. The method of claim 1, wherein said at least one anti-inflammatory agent is hydrophobic.

8. The method of claim 1, wherein said block copolymer further comprises at least one third block composed of a third plurality of backbone units covalently linked to one another, the third block comprises at least one additional moiety covalently attached, directly or via a linking moiety or group, to at least a portion of the third plurality of backbone units composing said third block, said additional moiety being a hydrophobic moiety that is different from said at least one anti-inflammatory agent or an additional hydrophilic moiety that is different from said hydrophilic moiety that comprises an amino acid sequence that is capable of interacting with an inflammatory biomarker.

9. The method of claim 1, wherein said block copolymer further comprises at least one backbone unit that has a labeling agent attached thereto.

10. The method of claim 1, wherein said block copolymer is represented by Formula I:
Q1-(L_{q1})-[(A1)(-L₁ₐ-D₁ₐ))ₘ₁-(A2(-L_{1b}-D_{1b}-D_{1b}))ₘ₂]_{z1}-[(B1(-L₂ₐ-P₁ₐ)ₙ₁-(B2(L_{2b}-P_{1b}) ₙ₂]_{z2}-[(C1(-L₃ₐ-M₁ₐ)ₓ₁-(C2(-L_{3b}-M_{1b})ₓ₂]_{z3}-[(E1-(L₄ₐ-Yₐ)_{y1}-(E2-(L_{4b}-Y_{1b})_{y2}]_{z4}-(L_{q2})-Q2 Formula I
wherein:
Q1 and Q2 are each independently a terminal backbone group;
L_{q1} and L_{q2} are each independently a linking moiety or group or absent;
z1 is a positive integer representing the number of said at least one first block(s) in the block copolymer;
[(A1(-L₁ₐ-D₁ₐ))ₘ₁-(A2(-L_{1b}-D_{1b}))ₘ₂] represents said at least one first block wherein:
A1 and A2 are each independently said first plurality of backbone units composing said first block;
L₁ₐ and L_{1b} are each independently a linking group or moiety or absent;
D₁ₐ and D_{1b} are each independently said anti-inflammatory agent;
(A1(-L₁ₐ-D₁ₐ)) represents a first portion of said first plurality of backbone units composing said first block;
m1 is a positive integer representing the number backbone units in said first portion of said first plurality of backbone units composing said first block:
(A2(-L_{1b}-D_{1b})) represents a second portion of said first plurality of backbone units composing said first block;
m2 is 0 or a positive integer representing the number backbone units in said second portion of said first plurality of backbone units composing said first block;
z2 is a positive integer representing the number of said at least one second block(s) in the block copolymer;
[(B1(-L₂ₐ-P₁ₐ)ₙ₁—(B2(L_{2b}-P_{1b})ₙ₂] represents said at least one first block wherein:
B1 and B2 are each independently said second plurality of backbone units composing said second block;
L₂ₐ and L_{2b} are each independently a linking group or moiety or absent:
P₁ₐ and P_{1b} are each independently said hydrophilic moiety that comprises an amino acid sequence that is capable of interacting with an inflammatory biomarker,
(B1(-L₂ₐ-P₁ₐ)) represents a first portion of said second plurality of backbone units composing said second block:
n1 is a positive integer representing the number backbone units in said first portion of said second plurality of backbone units composing said second block;
(B2(-L_{2b}-P_{1b})) represents a second portion of said second plurality of backbone units composing said second block;
n2 is 0 or a positive integer representing the number backbone units in said second portion of said second plurality of backbone units composing said second block;
z3 is 0 or a positive integer representing the number of at least one third block in the block copolymer;
[(C₁(-L₃ₐ-M₁ₐ))ₓ₁-(C₂(-L_{3b}-M_{1b}))ₓ₂] represents said at least one third block wherein:
C₁ and C₂ are each independently a third plurality of backbone units composing said third block;
L₃ₐ and L_{3b} are each independently a linking group or moiety or absent;
M₁ₐ and M_{1b} are each independently said additional moiety as defined in claim 8;
(C₁(-L₃ₐ-M₁ₐ)) represents a first portion of said third plurality of backbone units composing said third block;
x1 is a positive integer representing the number backbone units in said first portion of said third plurality of backbone units composing said third block;
(C₂(-L_{3b}-M_{1b})) represents a second portion of said third plurality of backbone units composing said third block;
x2 is 0 or a positive integer representing the number backbone units in said second portion of said third plurality of backbone units composing said third block;
z4 is 0 or a positive integer representing the number of at least one fourth block in the block copolymer;
[(E1(-L₄ₐ-Y₁ₐ))_{y1}-(E2(-L_{4b}-Y_{1b}))_{y2}] represents said at least one fourth block wherein:
E1 and E2 are each independently a fourth plurality of backbone units composing said fourth block;
L₁ₐ and L_{1b} are each independently a linking group or moiety or absent;
Y₁ₐ and Y_{1b} are each independently a labeling agent;
(E1(L₄ₐ-Y₁ₐ)) represents a first portion of said fourth plurality of backbone units composing said fourth block;
y1 is a positive integer representing the number backbone units in said first portion of said fourth plurality of backbone units composing said fourth block;
(E2(-L_{4b}-Y_{1b})) represents a second portion of said fourth plurality of backbone units composing said fourth block; and
y2 is 0 or a positive integer representing the number backbone units in said second portion of said fourth plurality of backbone units composing said fourth block,
wherein said at least one first block, said at least one second block, said at least one third block, if present, and said at least one fourth block, if present, are randomly arranged in the block co-polymer.

11. The method of claim 10, wherein:
m2, n2, x2 and y2 are independently 0 or a positive integer of from 1 to 100; and/or
m1 and n1 are independently a positive integer of from 1 to 100; and/or
x1 is a positive integer of from 1 to 100; and/or
y1 is a positive integer of from 1 to 10, or is 1; and/or
z1 and z2 are each independently a positive integer of from 1 to 10, or each is 1; and/or
a ratio of z1 to z2 ranges from 100:1 to 1:100; and/or
z3 is 0 or a positive integer of from 1 to 10, wherein when z3 is an integer of from 2 to 10, each of said third blocks can be the same or different; and/or
z4 is 0 or a positive integer of from 1 to 10, wherein when z4 is an integer of from 2 to 10, each of said fourth blocks can be the same or different, and wherein when z4 is a positive integer, the block copolymer is arranged such that at least one fourth block is adjacent to said at least one second block; and/or
Q1 and Q2 each independently comprises independently at least two of hydrogen, alkyl, cycloalkyl, alkenyl, alkoxy, thioalkoxy, aryloxy, amine, a heteroalicyclic, heteroaryl, aryl, a labeling agent and a therapeutically active agent; and/or
each of said linking moieties or groups L_{q1}, L_{q2}, L₁ₐ, L_{1b}, L₂ₐ, L_{2b}, L₃ₐ, L_{3b}, L₄ₐ and L_{4b} is independently selected from —O—, —S—, —NH—, —C(═O)—, —C(═O)O—, —C(═O)NH—, alkylene, alkoxy, aryloxy, thioalkoxy, cycloalkyl, heteroalicyclic, aryl and heteroaryl.

12. The method of claim 1, wherein each backbone unit of said first plurality of backbone units of said at least one first block, second plurality of backbone units of said at least one second block, third plurality of backbone units of said at least one third block, if present, and fourth plurality of backbone units of at least one said fourth block, if present, is independently a ROMP-polymerized monomer.

13. The method of claim 1, wherein said anti-inflammatory agent is dexamethasone or a therapeutically active derivative thereof.

14. The method of claim 1, wherein said hydrophilic moiety comprises an amino acid sequence as set forth in SEQ ID NOS: 1, 3, 4, 12, 14, and 16.

15. The method of claim 1, wherein the micellar particle forms a part of a pharmaceutical composition which further comprises a pharmaceutically acceptable carrier.

16. The method of claim 1, wherein the micellar particle or a pharmaceutical composition comprising same is locally administering to an inflamed tissue and/or to a nearby vicinity thereof.

17. The method of claim 1, wherein the micellar particle or a pharmaceutical composition comprising same is administered to the subject once during a time period of at least one day, at least one week, or at least one month.

18. A method of preparing a micellar particle comprising a plurality of block copolymers, at least a portion, or each, of said block copolymers comprising at least one first block composed of a first plurality of backbone units covalently linked to one another and at least one second block of a second plurality of backbone units covalently linked to one another,
wherein:
the first block comprises at least one anti-inflammatory agent covalently attached, directly or via a linking moiety or group, to at least a portion of the first plurality of backbone units composing said first block; and
the second block comprises at least one hydrophilic moiety covalently attached to at least a portion of the second plurality of backbone units composing said second block, said at least one hydrophilic moiety comprises an amino acid sequence that is capable of interacting with an inflammatory biomarker,
and wherein the micellar particle comprises a core comprising said first block and a hydrophilic shell comprising said second block,
the method comprising contacting the plurality of block copolymers with an aqueous solution.

19. The method of claim 18, wherein said block copolymer is represented by Formula I:
Q1-(L_{q1})-[(A1)(-L₁ₐ-D₁ₐ))ₘ₁-(A2(-L_{1b}-D_{1b}-D_{1b}))ₘ₂]_{z1}-[(B1(-L₂ₐ-P₁ₐ)ₙ₁-(B2(L_{2b}-P_{1b}) ₙ₂]_{z2}-[(C1(-L₃ₐ-M₁ₐ)ₓ₁-(C2(-L_{3b}-M_{1b})ₓ₂]_{z3}-[(E1-(L₄ₐ-Yₐ)_{y1}-(E2-(L_{4b}-Y_{1b})_{y2}]_{z4}-(L_{q2})-Q2 Formula I
wherein:
Q1 and Q2 are each independently a terminal backbone group;
L_{q1} and L_{q2} are each independently a linking moiety or group or absent;
z1 is a positive integer representing the number of said at least one first block(s) in the block copolymer;
[(A1(-L₁ₐ-D₁ₐ))ₘ₁-(A2(-L_{1b}-D_{1b}))ₘ₂] represents said at least one first block wherein:
A1 and A2 are each independently said first plurality of backbone units composing said first block;
L₁ₐ and L_{1b} are each independently a linking group or moiety or absent;
D₁ₐ and D_{1b} are each independently said anti-inflammatory agent;
(A1(-L₁ₐ-D₁ₐ)) represents a first portion of said first plurality of backbone units composing said first block;
m1 is a positive integer representing the number backbone units in said first portion of said first plurality of backbone units composing said first block;
(A2(-L_{1b}-D_{1b})) represents a second portion of said first plurality of backbone units composing said first block;
m2 is 0 or a positive integer representing the number backbone units in said second portion of said first plurality of backbone units composing said first block;
z2 is a positive integer representing the number of said at least one second block(s) in the block copolymer;
[(B1(-L₂ₐ-P₁ₐ)ₙ₁—(B2(L_{2b}-P_{1b})ₙ₂] represents said at least one first block wherein:
B1 and B2 are each independently said second plurality of backbone units composing said second block;
L₂ₐ and L_{2b} are each independently a linking group or moiety or absent;
P₁ₐ and P_{1b} are each independently said hydrophilic moiety that comprises an amino acid sequence that is capable of interacting with an inflammatory biomarker;
(B1(-L₂ₐ-P₁ₐ)) represents a first portion of said second plurality of backbone units composing said second block:
n1 is a positive integer representing the number backbone units in said first portion of said second plurality of backbone units composing said second block;
(B2(-L_{2b}-P_{1b})) represents a second portion of said second plurality of backbone units composing said second block;
n2 is 0 or a positive integer representing the number backbone units in said second portion of said second plurality of backbone units composing said second block;
z3 is 0 or a positive integer representing the number of at least one third block in the block copolymer;
[(C₁(-L₃ₐ-M₁ₐ))ₓ₁-(C₂(-L_{3b}-M_{1b}))ₓ₂] represents said at least one third block wherein:
C₁ and C₂ are each independently a third plurality of backbone units composing said third block;
L₃ₐ and L_{3b} are each independently a linking group or moiety or absent;
M₁ₐ and M_{1b} are each independently said additional moiety as defined in claim 8;
(C₁(-L₃ₐ-M₁ₐ)) represents a first portion of said third plurality of backbone units composing said third block;
x1 is a positive integer representing the number backbone units in said first portion of said third plurality of backbone units composing said third block:
(C₂(-L_{3b}-M_{1b})) represents a second portion of said third plurality of backbone units composing said third block;
x2 is 0 or a positive integer representing the number backbone units in said second portion of said third plurality of backbone units composing said third block;
z4 is 0 or a positive integer representing the number of at least one fourth block in the block copolymer;
[(E1(-L₄ₐ-Y₁ₐ))_{y1}-(E2(-L_{4b}-Y_{1b}))_{y2}] represents said at least one fourth block wherein:
E1 and E2 are each independently a fourth plurality of backbone units composing said fourth block;
L₄ₐ and L_{4b} are each independently a linking group or moiety or absent;
Y₁ₐ and Y_{1b} are each independently a labeling agent;
(E1(-L₄ₐ-Y₁ₐ)) represents a first portion of said fourth plurality of backbone units composing said fourth block;
y1 is a positive integer representing the number backbone units in said first portion of said fourth plurality of backbone units composing said fourth block;
(E2(-L_{4b}-Y_{1b})) represents a second portion of said fourth plurality of backbone units composing said fourth block; and
y2 is 0 or a positive integer representing the number backbone units in said second portion of said fourth plurality of backbone units composing said fourth block,
wherein said at least one first block, said at least one second block, said at least one third block, if present, and said at least one fourth block, if present, are randomly arranged in the block co-polymer.

20. The method of claim 19, wherein:
m2, n2, x2 and y2 are independently 0 or a positive integer of from 1 to 100; and/or
m1 and n1 are independently a positive integer of from 1 to 100; and/or
x1 is a positive integer of from 1 to 100; and/or
y1 is a positive integer of from 1 to 10, or is 1; and/or
z1 and z2 are each independently a positive integer of from 1 to 10, or each is 1; and/or
a ratio of z1 to z2 ranges from 100:1 to 1:100; and/or
z3 is 0 or a positive integer of from 1 to 10, wherein when z3 is an integer of from 2 to 10, each of said third blocks can be the same or different; and/or
z4 is 0 or a positive integer of from 1 to 10, wherein when z4 is an integer of from 2 to 10, each of said fourth blocks can be the same or different, and wherein when z4 is a positive integer, the block copolymer is arranged such that at least one fourth block is adjacent to said at least one second block; and/or
Q1 and Q2 each independently comprises independently at least two of hydrogen, alkyl, cycloalkyl, alkenyl, alkoxy, thioalkoxy, aryloxy, amine, a heteroalicyclic, heteroaryl, aryl, a labeling agent and a therapeutically active agent; and/or
each of said linking moieties or groups L_{q1}, L_{q2}, L₁ₐ, L_{1b}, L₂ₐ, L_{2b}, L₃ₐ, L_{3b}, L₄ₐ and L_{4b} is independently selected from —O—, —S—, —NH—, —C(═O)—, —C(═O)O—, —C(═O)NH—, alkylene, alkoxy, aryloxy, thioalkoxy, cycloalkyl, heteroalicyclic, aryl and heteroaryl.

21. A block copolymer comprising at least one first block composed of a first plurality of backbone units covalently linked to one another and at least one second block of a second plurality of backbone units covalently linked to one another,
wherein:
the first block comprises at least one anti-inflammatory agent covalently attached, directly or via a linking moiety or group, to at least a portion of the first plurality of backbone units composing said first block; and
the second block comprises at least one hydrophilic moiety covalently attached to at least a portion of the second plurality of backbone units composing said second block, said at least one hydrophilic moiety comprises an amino acid sequence that is capable of interacting with an inflammatory biomarker.

22. The block copolymer of claim 21, represented by Formula I:
Q1-(L_{q1})-[(A1)(-L₁ₐ-D₁ₐ))ₘ₁-(A2(-L_{1b}-D_{1b}-D_{1b}))ₘ₂]_{z1}-[(B1(-L₂ₐ-P₁ₐ)ₙ₁-(B2(L_{2b}-P_{1b}) ₙ₂]_{z2}-[(C1(-L₃ₐ-M₁ₐ)ₓ₁-(C2(-L_{3b}-M_{1b})ₓ₂]_{z3}-[(E1-(L₄ₐ-Yₐ)_{y1}-(E2-(L_{4b}-Y_{1b})_{y2}]_{z4}-(L_{q2})-Q2 Formula I
wherein:
Q1 and Q2 are each independently a terminal backbone group;
L_{q1} and L_{q2} are each independently a linking moiety or group or absent;
z1 is a positive integer representing the number of said at least one first block(s) in the block copolymer;
[(A1(-L₁ₐ-D₁ₐ))ₘ₁-(A2(-L_{1b}-D_{1b}))ₘ₂] represents said at least one first block wherein:
A1 and A2 are each independently said first plurality of backbone units composing said first block;
L₁ₐ and L_{1b} are each independently a linking group or moiety or absent;
D₁ₐ and D_{1b} are each independently said anti-inflammatory agent;
(A1(-L₁ₐ-D₁ₐ)) represents a first portion of said first plurality of backbone units composing said first block;
m1 is a positive integer representing the number backbone units in said first portion of said first plurality of backbone units composing said first block;
(A2(-L_{1b}-D_{1b})) represents a second portion of said first plurality of backbone units composing said first block;
m2 is 0 or a positive integer representing the number backbone units in said second portion of said first plurality of backbone units composing said first block;
z2 is a positive integer representing the number of said at least one second block(s) in the block copolymer;
[(B1(-L₂ₐ-P₁ₐ)ₙ₁—(B2(L_{2b}-P_{1b})ₙ₂] represents said at least one first block wherein:
B1 and B2 are each independently said second plurality of backbone units composing said second block;
L₂ₐ and L_{2b} are each independently a linking group or moiety or absent;
P₁ₐ and P_{1b} are each independently said hydrophilic moiety that comprises an amino acid sequence that is capable of interacting with an inflammatory biomarker,
(B1(-L₂ₐ-P₁ₐ)) represents a first portion of said second plurality of backbone units composing said second block;
n1 is a positive integer representing the number backbone units in said first portion of said second plurality of backbone units composing said second block;
(B2(-L_{2b}-P_{1b})) represents a second portion of said second plurality of backbone units composing said second block;
n2 is 0 or a positive integer representing the number backbone units in said second portion of said second plurality of backbone units composing said second block;
z3 is 0 or a positive integer representing the number of at least one third block in the block copolymer;
[(C₁(-L₃ₐ-M₁ₐ))ₓ₁-(C₂(-L_{3b}-M_{1b}))ₓ₂] represents said at least one third block wherein:
C₁ and C₂ are each independently a third plurality of backbone units composing said third block;
L₃ₐ and L_{3b} are each independently a linking group or moiety or absent;
M₁ₐ and M_{1b} are each independently said additional moiety as defined in claim 8:
(C₁(-L₃ₐ-M₁ₐ)) represents a first portion of said third plurality of backbone units composing said third block;
x1 is a positive integer representing the number backbone units in said first portion of said third plurality of backbone units composing said third block;
(C₂(-L_{3b}-M_{1b})) represents a second portion of said third plurality of backbone units composing said third block;
x2 is 0 or a positive integer representing the number backbone units in said second portion of said third plurality of backbone units composing said third block;
z4 is 0 or a positive integer representing the number of at least one fourth block in the block copolymer;
[(E(-L₄ₐ-Y₁ₐ))_{y1}-(E2(-L_{4b}-Y_{1b}))_{y2}] represents said at least one fourth block wherein:
E1 and E2 are each independently a fourth plurality of backbone units composing said fourth block;
L₄ₐ and L_{4b} are each independently a linking group or moiety or absent;
Y₁ₐ and Y_{1b} are each independently a labeling agent;
(E1(L₄ₐ-Y₁ₐ)) represents a first portion of said fourth plurality of backbone units composing said fourth block;
y1 is a positive integer representing the number backbone units in said first portion of said fourth plurality of backbone units composing said fourth block;
(E2(-L_{4b}-Y_{1b})) represents a second portion of said fourth plurality of backbone units composing said fourth block; and
y2 is 0 or a positive integer representing the number backbone units in said second portion of said fourth plurality of backbone units composing said fourth block,
wherein said at least one first block, said at least one second block, said at least one third block, if present, and said at least one fourth block, if present, are randomly arranged in the block co-polymer.

23. The block copolymer of claim 19, wherein:
m2, n2, x2 and y2 are independently 0 or a positive integer of from 1 to 100; and/or
m1 and n1 are independently a positive integer of from 1 to 100; and/or
x1 is a positive integer of from 1 to 100; and/or
y1 is a positive integer of from 1 to 10, or is 1; and/or
z1 and z2 are each independently a positive integer of from 1 to 10, or each is 1; and/or
a ratio of z1 to z2 ranges from 100:1 to 1:100; and/or
z3 is 0 or a positive integer of from 1 to 10, wherein when z3 is an integer of from 2 to 10,
each of said third blocks can be the same or different; and/or
z4 is 0 or a positive integer of from 1 to 10, wherein when z4 is an integer of from 2 to 10, each of said fourth blocks can be the same or different, and wherein when z4 is a positive integer, the block copolymer is arranged such that at least one fourth block is adjacent to said at least one second block; and/or
Q1 and Q2 each independently comprises independently at least two of hydrogen, alkyl, cycloalkyl, alkenyl, alkoxy, thioalkoxy, aryloxy, amine, a heteroalicyclic, heteroaryl, aryl, a labeling agent and a therapeutically active agent; and/or
each of said linking moieties or groups L_{q1}, L_{q2}, L₁ₐ, L_{1b}, L₂ₐ, L_{2b}, L₃ₐ, L_{3b}, L₄ₐ and L_{4b} is independently selected from —O—, —S—, —NH—, —C(═O)—, —C(═O)O—, —C(═O)NH—, alkylene, alkoxy, aryloxy, thioalkoxy, cycloalkyl, heteroalicyclic, aryl and heteroaryl.

24. A pharmaceutical composition comprising a micellar particle which comprises a block copolymer comprising at least one first block composed of a first plurality of backbone units covalently linked to one another and at least one second block of a second plurality of backbone units covalently linked to one another,
wherein:
the first block comprises at least one anti-inflammatory agent covalently attached, directly or via a linking moiety or group, to at least a portion of the first plurality of backbone units composing said first block; and
the second block comprises at least one hydrophilic moiety covalently attached to at least a portion of the second plurality of backbone units composing said second block, said at least one hydrophilic moiety comprises an amino acid sequence that is capable of interacting with an inflammatory biomarker,
and wherein the micellar particle comprises a core comprising said first block and a hydrophilic shell comprising said second block, the method comprising contacting the plurality of block copolymers with an aqueous solution,
and a pharmaceutically acceptable carrier, the composition being formulated for local administration at or a in close vicinity to, an inflamed tissue in the subject.

25. A polymeric aggregate formed upon contacting a plurality of micellar particles with an inflammatory marker, wherein at least one of said micellar particles comprises a block copolymer comprising at least one first block composed of a first plurality of backbone units covalently linked to one another and at least one second block of a second plurality of backbone units covalently linked to one another,
wherein:
the first block comprises at least one anti-inflammatory agent covalently attached, directly or via a linking moiety or group, to at least a portion of the first plurality of backbone units composing said first block; and
the second block comprises at least one hydrophilic moiety covalently attached to at least a portion of the second plurality of backbone units composing said second block, said at least one hydrophilic moiety comprises an amino acid sequence that is capable of interacting with an inflammatory biomarker,
and wherein the micellar particle comprises a core comprising said first block and a hydrophilic shell comprising said second block.
